SEQUENCE LISTING (130 sequences)

SEQ ID NO: 1 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 2 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 3 - protein, 17 aa, Artificial Sequence
  Description of Artificial Sequence Synthetic peptide
QIEYLAKQIVDNAIQQA

SEQ ID NO: 4 - protein, 21 aa, Artificial Sequence
  Description of Artificial Sequence Synthetic peptide
CGQIEYLAKQIVDNAIQQAGC

SEQ ID NO: 5 - protein, 50 aa, Artificial Sequence
  Description of Artificial Sequence Synthetic polypeptide
SLRECELYVQKHNIQALLKDSIVQLCTARPERPMAFLREYFERLEKEEAK

SEQ ID NO: 6 - protein, 55 aa, Artificial Sequence
  Description of Artificial Sequence Synthetic polypeptide
MSCGGSLRECELYVQKHNIQALLKDSIVQLCTARPERPMAFLREYFERLEKEEAK

SEQ ID NO: 7 - protein, 23 aa, Artificial Sequence
  Description of Artificial Sequence Synthetic peptide
CGFEELAWKIAKMIWSDVFQQGC

SEQ ID NO: 8 - protein, 51 aa, Homo sapiens
SLRECELYVQKHNIQALLKDVSIVQLCTARPERPMAFLREYFEKLEKEEAK

SEQ ID NO: 9 - protein, 54 aa, Homo sapiens
SLKGCELYVQLHGIQQVLKDCIVHLCISKPERPMKFLREHFEKLEKEENRQILA

SEQ ID NO: 10 - protein, 44 aa, Homo sapiens
SHIQIPPGLTELLQGYTVEVGQQPPDLVDFAVEYFTRLREARRQ

SEQ ID NO: 11 - protein, 44 aa, Homo sapiens
SIEIPAGLTELLQGFTVEVLRHQPADLLEFALQHFTRLQQENER

SEQ ID NO: 12 - protein, 44 aa, Artificial Sequence
  Description of Artificial Sequence Synthetic polypeptide
THIQIPPGLTELLQGYTVEVLRQQPPDLVEFAVEYFTRLREARA

SEQ ID NO: 13 - protein, 44 aa, Artificial Sequence
  Description of Artificial Sequence Synthetic polypeptide
SKIQIPPGLTELLQGYTVEVLRQQPPDLVEFAVEYFTRLREARA

SEQ ID NO: 14 - protein, 44 aa, Artificial Sequence
  Description of Artificial Sequence Synthetic polypeptide
SRIQIPPGLTELLQGYTVEVLRQQPPDLVEFAVEYFTRLREARA

SEQ ID NO: 15 - protein, 44 aa, Artificial Sequence
  Description of Artificial Sequence Synthetic polypeptide
SHINIPPGLTELLQGYTVEVLRQQPPDLVEFAVEYFTRLREARA

SEQ ID NO: 16 - protein, 44 aa, Artificial Sequence
  Description of Artificial Sequence Synthetic polypeptide
SHIQIPPALTELLQGYTVEVLRQQPPDLVEFAVEYFTRLREARA

SEQ ID NO: 17 - protein, 44 aa, Artificial Sequence
  Description of Artificial Sequence Synthetic polypeptide
SHIQIPPGLSELLQGYTVEVLRQQPPDLVEFAVEYFTRLREARA

SEQ ID NO: 18 - protein, 44 aa, Artificial Sequence
  Description of Artificial Sequence Synthetic polypeptide
SHIQIPPGLTDLLQGYTVEVLRQQPPDLVEFAVEYFTRLREARA

SEQ ID NO: 19 - protein, 44 aa, Artificial Sequence
  Description of Artificial Sequence Synthetic polypeptide
SHIQIPPGLTELLNGYTVEVLRQQPPDLVEFAVEYFTRLREARA

SEQ ID NO: 20 - protein, 44 aa, Artificial Sequence
  Description of Artificial Sequence Synthetic polypeptide
SHIQIPPGLTELLQAYTVEVLRQQPPDLVEFAVEYFTRLREARA

SEQ ID NO: 21 - protein, 44 aa, Artificial Sequence
  Description of Artificial Sequence Synthetic polypeptide
SHIQIPPGLTELLQGYSVEVLRQQPPDLVEFAVEYFTRLREARA

SEQ ID NO: 22 - protein, 44 aa, Artificial Sequence
  Description of Artificial Sequence Synthetic polypeptide
SHIQIPPGLTELLQGYTVDVLRQQPPDLVEFAVEYFTRLREARA

SEQ ID NO: 23 - protein, 44 aa, Artificial Sequence
  Description of Artificial Sequence Synthetic polypeptide
SHIQIPPGLTELLQGYTVEVLKQQPPDLVEFAVEYFTRLREARA

SEQ ID NO: 24 - protein, 44 aa, Artificial Sequence
  Description of Artificial Sequence Synthetic polypeptide
SHIQIPPGLTELLQGYTVEVLRNQPPDLVEFAVEYFTRLREARA

SEQ ID NO: 25 - protein, 44 aa, Artificial Sequence
  Description of Artificial Sequence Synthetic polypeptide
SHIQIPPGLTELLQGYTVEVLRQNPPDLVEFAVEYFTRLREARA

SEQ ID NO: 26 - protein, 44 aa, Artificial Sequence
  Description of Artificial Sequence Synthetic polypeptide
SHIQIPPGLTELLQGYTVEVLRQQPPELVEFAVEYFTRLREARA

SEQ ID NO: 27 - protein, 44 aa, Artificial Sequence
  Description of Artificial Sequence Synthetic polypeptide
SHIQIPPGLTELLQGYTVEVLRQQPPDLVDFAVEYFTRLREARA

SEQ ID NO: 28 - protein, 44 aa, Artificial Sequence
  Description of Artificial Sequence Synthetic polypeptide
SHIQIPPGLTELLQGYTVEVLRQQPPDLVEFLVEYFTRLREARA

SEQ ID NO: 29 - protein, 44 aa, Artificial Sequence
  Description of Artificial Sequence Synthetic polypeptide
SHIQIPPGLTELLQGYTVEVLRQQPPDLVEFIVEYFTRLREARA

SEQ ID NO: 30 - protein, 44 aa, Artificial Sequence
  Description of Artificial Sequence Synthetic polypeptide
SHIQIPPGLTELLQGYTVEVLRQQPPDLVEFVVEYFTRLREARA

SEQ ID NO: 31 - protein, 44 aa, Artificial Sequence
  Description of Artificial Sequence Synthetic polypeptide
SHIQIPPGLTELLQGYTVEVLRQQPPDLVEFAVDYFTRLREARA

SEQ ID NO: 32 - protein, 17 aa, Artificial Sequence
  Description of Artificial Sequence Synthetic peptide
NIEYLAKQIVDNAIQQA

SEQ ID NO: 33 - protein, 17 aa, Artificial Sequence
  Description of Artificial Sequence Synthetic peptide
QLEYLAKQIVDNAIQQA

SEQ ID NO: 34 - protein, 17 aa, Artificial Sequence
  Description of Artificial Sequence Synthetic peptide
QVEYLAKQIVDNAIQQA

SEQ ID NO: 35 - protein, 17 aa, Artificial Sequence
  Description of Artificial Sequence Synthetic peptide
QIDYLAKQIVDNAIQQA

SEQ ID NO: 36 - protein, 17 aa, Artificial Sequence
  Description of Artificial Sequence Synthetic peptide
QIEFLAKQIVDNAIQQA

SEQ ID NO: 37 - protein, 17 aa, Artificial Sequence
  Description of Artificial Sequence Synthetic peptide
QIETLAKQIVDNAIQQA

SEQ ID NO: 38 - protein, 17 aa, Artificial Sequence
  Description of Artificial Sequence Synthetic peptide
QIESLAKQIVDNAIQQA

SEQ ID NO: 39 - protein, 17 aa, Artificial Sequence
  Description of Artificial Sequence Synthetic peptide
QIEYIAKQIVDNAIQQA

SEQ ID NO: 40 - protein, 17 aa, Artificial Sequence
  Description of Artificial Sequence Synthetic peptide
QIEYVAKQIVDNAIQQA

SEQ ID NO: 41 - protein, 17 aa, Artificial Sequence
  Description of Artificial Sequence Synthetic peptide
QIEYLARQIVDNAIQQA

SEQ ID NO: 42 - protein, 17 aa, Artificial Sequence
  Description of Artificial Sequence Synthetic peptide
QIEYLAKNIVDNAIQQA

SEQ ID NO: 43 - protein, 17 aa, Artificial Sequence
  Description of Artificial Sequence Synthetic peptide
QIEYLAKQIVENAIQQA

SEQ ID NO: 44 - protein, 17 aa, Artificial Sequence
  Description of Artificial Sequence Synthetic peptide
QIEYLAKQIVDQAIQQA

SEQ ID NO: 45 - protein, 17 aa, Artificial Sequence
  Description of Artificial Sequence Synthetic peptide
QIEYLAKQIVDNAINQA

SEQ ID NO: 46 - protein, 17 aa, Artificial Sequence
  Description of Artificial Sequence Synthetic peptide
QIEYLAKQIVDNAIQNA

SEQ ID NO: 47 - protein, 17 aa, Artificial Sequence
  Description of Artificial Sequence Synthetic peptide
QIEYLAKQIVDNAIQQL

SEQ ID NO: 48 - protein, 17 aa, Artificial Sequence
  Description of Artificial Sequence Synthetic peptide
QIEYLAKQIVDNAIQQI

SEQ ID NO: 49 - protein, 17 aa, Artificial Sequence
  Description of Artificial Sequence Synthetic peptide
QIEYLAKQIVDNAIQQV

SEQ ID NO: 50 - protein, 17 aa, Artificial Sequence
  Description of Artificial Sequence Synthetic peptide
QIEYVAKQIVDYAIHQA

SEQ ID NO: 51 - protein, 17 aa, Artificial Sequence
  Description of Artificial Sequence Synthetic peptide
QIEYKAKQIVDHAIHQA

SEQ ID NO: 52 - protein, 17 aa, Artificial Sequence
  Description of Artificial Sequence Synthetic peptide
QIEYHAKQIVDHAIHQA

SEQ ID NO: 53 - protein, 17 aa, Artificial Sequence
  Description of Artificial Sequence Synthetic peptide
QIEYVAKQIVDHAIHQA

SEQ ID NO: 54 - protein, 18 aa, Artificial Sequence
  Description of Artificial Sequence Synthetic peptide
PLEYQAGLLVQNAIQQAI

SEQ ID NO: 55 - protein, 18 aa, Artificial Sequence
  Description of Artificial Sequence Synthetic peptide
LLIETASSLVKNAIQLSI

SEQ ID NO: 56 - protein, 18 aa, Artificial Sequence
  Description of Artificial Sequence Synthetic peptide
LIEEAASRIVDAVIEQVK

SEQ ID NO: 57 - protein, 18 aa, Artificial Sequence
  Description of Artificial Sequence Synthetic peptide
ALYQFADRFSELVISEAL

SEQ ID NO: 58 - protein, 17 aa, Artificial Sequence
  Description of Artificial Sequence Synthetic peptide
LEQVANQLADQIIKEAT

SEQ ID NO: 59 - protein, 17 aa, Artificial Sequence
  Description of Artificial Sequence Synthetic peptide
FEELAWKIAKMIWSDVF

SEQ ID NO: 60 - protein, 18 aa, Artificial Sequence
  Description of Artificial Sequence Synthetic peptide
ELVRLSKRLVENAVLKAV

SEQ ID NO: 61 - protein, 18 aa, Artificial Sequence
  Description of Artificial Sequence Synthetic peptide
TAEEVSARIVQVVTAEAV

SEQ ID NO: 62 - protein, 18 aa, Artificial Sequence
  Description of Artificial Sequence Synthetic peptide
QIKQAAFQLISQVILEAT

SEQ ID NO: 63 - protein, 16 aa, Artificial Sequence
  Description of Artificial Sequence Synthetic peptide
LAWKIAKMIVSDVMQQ

SEQ ID NO: 64 - protein, 24 aa, Artificial Sequence
  Description of Artificial Sequence Synthetic peptide
DLIEEAASRIVDAVIEQVKAAGAY

SEQ ID NO: 65 - protein, 18 aa, Artificial Sequence
  Description of Artificial Sequence Synthetic peptide
LEQYANQLADQIIKEATE

SEQ ID NO: 66 - protein, 20 aa, Artificial Sequence
  Description of Artificial Sequence Synthetic peptide
FEELAWKIAKMIWSDVFQQC

SEQ ID NO: 67 - protein, 17 aa, Artificial Sequence
  Description of Artificial Sequence Synthetic peptide
QIEYLAKQIPDNAIQQA

SEQ ID NO: 68 - protein, 25 aa, Artificial Sequence
  Description of Artificial Sequence Synthetic peptide
KGADLIEEAASRIVDAVIEQVKAAG

SEQ ID NO: 69 - protein, 25 aa, Artificial Sequence
  Description of Artificial Sequence Synthetic peptide
KGADLIEEAASRIPDAPIEQVKAAG

SEQ ID NO: 70 - protein, 25 aa, Artificial Sequence
  Description of Artificial Sequence Synthetic peptide
PEDAELVRLSKRLVENAVLKAVQQY

SEQ ID NO: 71 - protein, 25 aa, Artificial Sequence
  Description of Artificial Sequence Synthetic peptide
PEDAELVRTSKRLVENAVLKAVQQY

SEQ ID NO: 72 - protein, 25 aa, Artificial Sequence
  Description of Artificial Sequence Synthetic peptide
PEDAELVRLSKRDVENAVLKAVQQY

SEQ ID NO: 73 - protein, 25 aa, Artificial Sequence
  Description of Artificial Sequence Synthetic peptide
PEDAELVRLSKRLPENAVLKAVQQY

SEQ ID NO: 74 - protein, 25 aa, Artificial Sequence
  Description of Artificial Sequence Synthetic peptide
PEDAELVRLSKRLPENAPLKAVQQY

SEQ ID NO: 75 - protein, 25 aa, Artificial Sequence
  Description of Artificial Sequence Synthetic peptide
PEDAELVRLSKRLVENAVEKAVQQY

SEQ ID NO: 76 - protein, 25 aa, Artificial Sequence
  Description of Artificial Sequence Synthetic peptide
EEGLDRNEEIKRAAFQIISQVISEA

SEQ ID NO: 77 - protein, 25 aa, Artificial Sequence
  Description of Artificial Sequence Synthetic peptide
LVDDPLEYQAGLLVQNAIQQAIAEQ

SEQ ID NO: 78 - protein, 25 aa, Artificial Sequence
  Description of Artificial Sequence Synthetic peptide
QYETLLIETASSLVKNAIQLSIEQL

SEQ ID NO: 79 - protein, 25 aa, Artificial Sequence
  Description of Artificial Sequence Synthetic peptide
LEKQYQEQLEEEVAKVIVSMSIAFA

SEQ ID NO: 80 - protein, 25 aa, Artificial Sequence
  Description of Artificial Sequence Synthetic peptide
NTDEAQEELAWKIAKMIVSDIMQQA

SEQ ID NO: 81 - protein, 25 aa, Artificial Sequence
  Description of Artificial Sequence Synthetic peptide
VNLDKKAVLAEKIVAEAIEKAEREL

SEQ ID NO: 82 - protein, 25 aa, Artificial Sequence
  Description of Artificial Sequence Synthetic peptide
NGILELETKSSKLVQNIIQTAVDQF

SEQ ID NO: 83 - protein, 25 aa, Artificial Sequence
  Description of Artificial Sequence Synthetic peptide
TQDKNYEDELTQVALALVEDVINYA

SEQ ID NO: 84 - protein, 25 aa, Artificial Sequence
  Description of Artificial Sequence Synthetic peptide
ETSAKDNINIEEAARFLVEKILVNH

SEQ ID NO: 85 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 44 aa, Artificial Sequence
  Description of Artificial Sequence Synthetic polypeptide
XXIXIPPXLXXLLXXYXVXVLXXXPPXLVXFXVXYFXXLXXXXX

SEQ ID NO: 88 - protein, 17 aa, Artificial Sequence
  Description of Artificial Sequence Synthetic peptide
XXXXXAXXIVXXAIXXX

SEQ ID NO: 89 - protein, 44 aa, Artificial Sequence
  Description of Artificial Sequence Synthetic polypeptide
XHIXIPPGLXELLQGYTXEVLRXQPPDLVEFAXXYFXXLXEXRX

SEQ ID NO: 90 - protein, 4 aa, Artificial Sequence
  Description of Artificial Sequence Synthetic peptide
FKYK

SEQ ID NO: 91 - DNA, 21 bp, Artificial Sequence
  Description of Artificial Sequence Synthetic oligonucleotide
aatgcggcggtggtgacagta

SEQ ID NO: 92 - DNA, 21 bp, Artificial Sequence
  Description of Artificial Sequence Synthetic oligonucleotide
aagctcagcacacagaaagac

SEQ ID NO: 93 - DNA, 21 bp, Artificial Sequence
  Description of Artificial Sequence Synthetic oligonucleotide
uaaaaucuuccugcccacctt

SEQ ID NO: 94 - DNA, 21 bp, Artificial Sequence
  Description of Artificial Sequence Synthetic oligonucleotide
ggaagcuguuggcugaaaatt

SEQ ID NO: 95 - RNA, 21 nt, Artificial Sequence
  Description of Artificial Sequence Synthetic oligonucleotide
aagaccagccucuuugcccag

SEQ ID NO: 96 - DNA, 19 bp, Artificial Sequence
  Description of Artificial Sequence Synthetic oligonucleotide
ggaccaggcagaaaacgag

SEQ ID NO: 97 - RNA, 17 nt, Artificial Sequence
  Description of Artificial Sequence Synthetic oligonucleotide
cuaucaggaugacgcgg

SEQ ID NO: 98 - RNA, 21 nt, Artificial Sequence
  Description of Artificial Sequence Synthetic oligonucleotide
ugacacaggcaggcuugacuu

SEQ ID NO: 99 - DNA, 19 bp, Artificial Sequence
  Description of Artificial Sequence Synthetic oligonucleotide
ggtgaagaagggcgtccaa

SEQ ID NO: 100 - DNA, 60 bp, Artificial Sequence
  Description of Artificial Sequence Synthetic oligonucleotide
gatccgttggagctgttggcgtagttcaagagactcgccaacagctccaacttttggaaa

SEQ ID NO: 101 - DNA, 20 bp, Artificial Sequence
  Description of Artificial Sequence Synthetic oligonucleotide
aggtggtgttaacagcagag

SEQ ID NO: 102 - DNA, 21 bp, Artificial Sequence
  Description of Artificial Sequence Synthetic oligonucleotide
aaggtggagcaagcggtggag

SEQ ID NO: 103 - DNA, 21 bp, Artificial Sequence
  Description of Artificial Sequence Synthetic oligonucleotide
aaggagttgaaggccgacaaa

SEQ ID NO: 104 - DNA, 21 bp, Artificial Sequence
  Description of Artificial Sequence Synthetic oligonucleotide
uauggagcugcagaggaugtt

SEQ ID NO: 105 - DNA, 49 bp, Artificial Sequence
  Description of Artificial Sequence Synthetic oligonucleotide
tttgaatatctgtgctgagaacacagttctcagcacagatattcttttt

SEQ ID NO: 106 - DNA, 29 bp, Artificial Sequence
  Description of Artificial Sequence Synthetic oligonucleotide
aatgagaaaagcaaaaggtgccctgtctc

SEQ ID NO: 107 - RNA, 21 nt, Artificial Sequence
  Description of Artificial Sequence Synthetic oligonucleotide
aaucaucaucaagaaagggca

SEQ ID NO: 108 - DNA, 21 bp, Artificial Sequence
  Description of Artificial Sequence Synthetic oligonucleotide
augacugucaggauguugctt

SEQ ID NO: 109 - RNA, 21 nt, Artificial Sequence
  Description of Artificial Sequence Synthetic oligonucleotide
gaacgaauccugaagacaucu

SEQ ID NO: 110 - DNA, 29 bp, Artificial Sequence
  Description of Artificial Sequence Synthetic oligonucleotide
aagcctggctacagcaatatgcctgtctc

SEQ ID NO: 111 - DNA, 21 bp, Artificial Sequence
  Description of Artificial Sequence Synthetic oligonucleotide
ugaccaucaccgaguuuautt

SEQ ID NO: 112 - DNA, 21 bp, Artificial Sequence
  Description of Artificial Sequence Synthetic oligonucleotide
aagtcggacgcaacagagaaa

SEQ ID NO: 113 - DNA, 21 bp, Artificial Sequence
  Description of Artificial Sequence Synthetic oligonucleotide
cuaccuuucuacggacgugtt

SEQ ID NO: 114 - DNA, 21 bp, Artificial Sequence
  Description of Artificial Sequence Synthetic oligonucleotide
ctgcctaaggcggatttgaat

SEQ ID NO: 115 - DNA, 21 bp, Artificial Sequence
  Description of Artificial Sequence Synthetic oligonucleotide
ttauuccuucuucgggaaguc

SEQ ID NO: 116 - DNA, 21 bp, Artificial Sequence
  Description of Artificial Sequence Synthetic oligonucleotide
aaccttctggaacccgcccac

SEQ ID NO: 117 - DNA, 19 bp, Artificial Sequence
  Description of Artificial Sequence Synthetic oligonucleotide
gagcatcttcgagcaagaa

SEQ ID NO: 118 - DNA, 19 bp, Artificial Sequence
  Description of Artificial Sequence Synthetic oligonucleotide
catgtggcaccgtttgcct

SEQ ID NO: 119 - DNA, 21 bp, Artificial Sequence
  Description of Artificial Sequence Synthetic oligonucleotide
aactaccagaaaggtatacct

SEQ ID NO: 120 - DNA, 21 bp, Artificial Sequence
  Description of Artificial Sequence Synthetic oligonucleotide
ucacaguguccuuuauguatt

SEQ ID NO: 121 - DNA, 21 bp, Artificial Sequence
  Description of Artificial Sequence Synthetic oligonucleotide
gcaugaaccggaggcccautt

SEQ ID NO: 122 - protein, 16 aa, Artificial Sequence
  Description of Artificial Sequence Synthetic peptide
EFPKPSTPPGSSGGAP

SEQ ID NO: 123 - protein, 11 aa, Artificial Sequence
  Description of Artificial Sequence Synthetic peptide
ACSSSPSKHCG

SEQ ID NO: 124 - protein, 8 aa, Artificial Sequence
  Description of Artificial Sequence Synthetic peptide
FCIGRLCG

SEQ ID NO: 125 - protein, 12 aa, Artificial Sequence
  Description of Artificial Sequence Synthetic peptide
GRKKRRNRRRCG

SEQ ID NO: 126 - DNA, 19 bp, Artificial Sequence
  Description of Artificial Sequence Synthetic oligonucleotide
ccggacagttccatgtata

SEQ ID NO: 127 - protein, 4 aa, Artificial Sequence
  Description of Artificial Sequence Synthetic peptide
PKSC

SEQ ID NO: 128 - protein, 55 aa, Artificial Sequence
  Description of Artificial Sequence Synthetic polypeptide
GSGGGGSGGGGSHIQIPPGLTELLQGYTVEVLRQQPPDLVEFAVEYFTRLREARA

SEQ ID NO: 129 - protein, 29 aa, Artificial Sequence
  Description of Artificial Sequence Synthetic peptide
GSGGGGSGGGGSQIEYLAKQIVDNAIQQA

SEQ ID NO: 130 - DNA, 2208 bp, Artificial Sequence
  Description of Artificial Sequence Synthetic polynucleotide
ggatcccgcaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgagttccctaaacccagcactccacccggatcttccggcggcgctccctgtggccagatcgagtacctggccaagcagatcgtggacaacgccatccagcaggccgggtgctagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaattcccacttctaggggttcgactggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaatttcgaccaattctcatgtttgacagcttatcatcgctgcactccgcccgaaaagtgcgctcggctctgccaaggacgcggggcgcgtgactatgcgtgggctggagcaaccgcctgctgggtgcaaaccctttgcgcccggactcgtccaacgactataaagagggcaggctgtcctctaagcgtcaccacgacttcaacgtcctgagtaccttctcctcacttactccgtagctccagcttcaccagatccctcgag

(19) United States
(12) Patent Application Publication
(10) Pub. No.: US 2014/0212425 A1
(43) Pub. Date: Jul. 31, 2014

(54) THERAPEUTIC USE OF ANTI-CD22 ANTIBODIES FOR INDUCING TROGOCYTOSIS

(71) Applicant: IMMUNOMEDICS, INC., MORRIS PLAINS, NJ (US)

(72) Inventors: Chien-Hsing Chang, Downingtown, PA (US); David M. Goldenberg, Mendham, NJ (US); Hans J. Hansen, Picayune, MS (US); Edmund A. Rossi, Woodland Park, NJ (US)

(73) Assignee: IMMUNOMEDICS, INC., MORRIS PLAINS, NJ (US)

(21) Appl. No.: 14/243,512

(22) Filed: Apr. 2, 2014

Related U.S. Application Data
(63) Continuation-in-part of application No. 13/693,476, filed on Dec. 4, 2012.
(60) Provisional application No. 61/566,828, filed on Dec. 5, 2011.
(60) Provisional application No. 61/609,075, filed on Mar. 9, 2012.
(60) Provisional application No. 61/682,508, filed on Aug. 13, 2012.
(60) Provisional application No. 61/718,226, filed on Oct. 25, 2012.
(60) Provisional application No. 61/808,005, filed on Apr. 3, 2013.
(60) Provisional application No. 61/832,558, filed on Jun. 7, 2013.
(60) Provisional application No. 61/941,100, filed on Feb. 18, 2014.

(51) Int. Cl. C07K 16/28; A61K 45/06; C07K 16/46; A61K 39/395

(52) U.S. Cl. CPC C07K 16/2851; A61K 39/3955; A61K 39/39558; A61K 45/06; C07K 16/468; C07K 16/2803; C07K 16/2887
USPC 424/136.1; 424/173.1; 424/133.1; 530/387.9

(57) ABSTRACT

Disclosed are methods and compositions of anti-B cell antibodies, preferably anti-CD22 antibodies, for diagnosis, prognosis and therapy of B-cell associated diseases, such as B-cell malignancies, autoimmune disease and immune dysfunction disease. Preferably, the antibodies induce trogocytosis of B-cell antigens, such as CD19, CD20, CD21, CD22, CD79b, CD44, CD62L, or β7-integrin. Trogocytosis may play a significant role in determining antibody efficacy, disease responsiveness and prognosis of therapeutic intervention and trogocytosis-dependent responses may be monitored by measuring the levels of trogocytosis of one or more B-cell surface antigens induced by the bispecific antibody.

### RELATED APPLICATIONS

The present application claims the benefit under 35 U.S.C. 119(e) of Provisional U.S. Patent Application Ser. Nos. 61/808,005, filed Apr. 3, 2013, 61/832,558, filed Jun. 7, 2013 and 61/941,100, filed Feb. 18, 2014. This application is a continuation-in-part of U.S. patent application Ser. No. 13/693,476, filed Dec. 4, 2012, which claimed the benefit under 35 U.S.C. 119(e) of Provisional U.S. Patent Application Ser. Nos. 61/566,828, filed Dec. 5, 2011; 61/609,075, filed Mar. 9, 2012; 61/682,508, filed Aug. 13, 2012; and 61/718,226, filed Oct. 25, 2012, each priority application incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Mar. 26, 2014, is named IMM338US1_SL.txt and is 57,483 bytes in size.

### FIELD OF THE INVENTION

The present invention concerns compositions and methods of use of antibodies against B-cell surface markers, such as CD19, CD20, CD21, CD22, CD79b, CD44, CD62L, β7-integrin or B-cell receptor (BCR). Preferably, the antibody is an anti-CD22 antibody. More preferably, the anti-B-cell antibody induces trogocytosis of multiple surface markers, which include, but are not limited to, CD19, CD20, CD21, CD22, CD79b, CD44, CD62L and β7-integrin on normal, autoimmune (e.g., lupus), and malignant B cells (donor cells) via leukocytes, including monocytes, NK cells and granulocytes (recipient cells). In alternative embodiments, the anti-B-cell antibody may be a bispecific antibody, with multiple specificities against B-cell antigens, or one specificity against a B-cell antigen and a second specificity against other disease-associated antigens, such as TNF-alpha, IL6 or CD3. More preferably, the bispecific antibody comprises at least one anti-CD22 antibody or fragment thereof and at least one anti-CD20 antibody or fragment thereof. Most preferably, the antibody efficacy, disease cell responsiveness and/or prognosis for disease progression are a function of trogocytosis induced by such antibodies. The trogocytosis-inducing antibody may be used alone, or in combination with other agents, which include one or more different antibodies that may or may not have trogocytosis-inducing activity. Where a combination of two antibodies is desirable, a bispecific antibody derived from the two antibodies of interest may be used in lieu of a combination of such antibodies. Bispecific antibodies are preferred to administration of combinations of separate antibodies, due to cost and convenience. However, where combinations of separate antibodies provide improved safety or efficacy, the combination may be utilized. One preferred form of the bispecific antibody is a hexavalent antibody (HexAb) that is made as a DOCK-AND-LOCK™ complex. Further, a bispecific antibody capable of bridging the donor and recipient cells may not require the presence of Fc for trogocytosis. The compositions and methods are of use in therapy and/or detection, diagnosis or prognosis of various disease states, including but not limited to autoimmune diseases, immune dysfunction diseases and cancers.

### BACKGROUND

Trogocytosis (also referred to as shaving in the literature) is a process by which transfer of membrane-bound proteins and membrane components occur between two different types of live cells associated to form an immunological synapse. As a result, the membrane-bound proteins and membrane components are transferred from the donor cells to the recipient cells. Both unidirectional and bidirectional trogocytosis between the two interacting cells may occur. One prominent example of trogocytosis is the extraction of surface antigens from antigen-presenting cells (APCs) by T cells (Joly & Hudrisier, 2003, Nat Immunol 4:85). The process involves transfer of plasma membrane fragments from the APC to the lymphocyte (Joly & Hudrisier, 2003). Intercellular transfer of T cell surface molecules to APCs has also been reported (Nolte-'t Hoen et al, 2004, Eur J Immunol 34: 3115-25; Busch et al 2008, J Immunol 181: 3965-73) via mechanisms that may include trogocytosis, exosomes and ectodomain shedding (Busch et al 2008, ibid). Trogocytosis can also occur between natural killer (NK) cells and tumors and can convert activated NK cells into suppressor cells, via uptake of the immunosuppressive HLA-G molecule, which protects the tumor cells from cytolysis (Caumartin et al., 2007, EMBO J. 26:423-30). CD4+ and CD8+ T cells can, respectively, acquire MHC Class II and MHC Class 1 molecules from APCs in an antigen-specific manner (Caumartin et al., 2007). Trogocytosis of HLA-DR, CD80 and HLA-G1 from APCs to T cells has been shown to occur in humans (Caumartin et al., 2007). After acquiring HLA-DR and CD80, T cells stimulated resting T cells in an antigen-specific manner, acting as APCs themselves (Caumartin et al., 2007). More generally, trogocytosis may act to regulate immune system responsiveness to disease-associated antigens and can either stimulate or suppress immune response (Ahmed et al., 2008, Cell Mol Immunol 5:261-69).

The effects of trogocytosis on therapeutic antibody responsiveness and the induction of trogocytosis by therapeutic antibodies remain poorly understood. It has been suggested that induction of trogocytosis by excess amounts of rituximab may result in removal of rituximab-CD20 complexes from tumor cell surfaces by monocytes, producing antigenic modulation (shaving) and rituximab-resistant tumor cells (Beum et al., 2006, J Immunol 176:2600-8). Thus, use of lower, more frequent doses of rituximab to reduce antigen shaving has been suggested (Beum et al., 2006). Transfer of rituximab/CD20 complexes to monocytes is mediated by FcγR and it has also been suggested that polymorphisms in FcγRII and FcγRIII may affect the degree of antibody-induced shaving and predict responsiveness to antibody therapy (Beum et al., 2006). In this regard, use of antibodies or other inhibitors that block trogocytosis may enhance efficacy and reduce tumor cell escape from cytotoxicity (Beum et al., 2006). On the other hand, the functional consequences of antibody-mediated trogocytosis to confer therapeutic benefits are less explored.

A need exists in the art for a better understanding of the induction of trogocytosis by therapeutic anti-B-cell antibodies, the effect of trogocytosis on antigen shaving, and the effects of trogocytosis and shaving on therapeutic efficacy, target cell susceptibility, and immune system responses in various disease states.

### SUMMARY

The present invention concerns compositions and methods of use of antibodies against B-cell surface markers, such as such as CD19, CD20, CD21, CD22, CD79b, CD44, CD62L and/or β7-integrin. Preferably, the antibody is an anti-CD22 antibody. More preferably, the antibody induces trogocytosis of multiple surface markers, which include, but are not limited to, CD19, CD20, CD21, CD22, CD79b, CD44, CD62L and β7-integrin on normal, autoimmune (e.g., lupus), and malignant B cells via monocytes, NK cells and granulocytes. Most preferably, the antibody displays little or negligible direct cytotoxicity to normal B cells based on an in vitro cell proliferation assay that shows less than 20% growth inhibition when compared with untreated control, CD19, CD20, CD21, CD22, CD79b, CD44, CD62L and β7-integrin on normal, autoimmune (e.g., lupus), and malignant B cells via monocytes, NK cells and granulocytes. One example of a preferred anti-CD22 antibody is epratuzumab, which induces trogocytosis without incurring direct cytotoxicity to B cells, thus providing an unexpected and substantial advantage in treating autoimmune diseases, such as systemic lupus erythematosus (SLE), ANCA-associated vasculitides, and other autoimmune diseases.

In certain embodiments, administration of an antibody against a selective B cell marker, such as an anti-CD22 antibody, induces trogocytosis in B cells, resulting in decreased levels of CD19, CD20, CD21, CD22, CD79b, CD44, CD62L and β7-integrin on the surface of affected B cells. B cell antigens, particularly CD19, inhibits B cell activation in response to T cell-dependent antigens and has a therapeutic effect on autoimmune and immune dysfunction diseases, which are mediated at least in part by B cell activation. In certain alternative embodiments, an affibody or fynomer fused to a human Fc may be used in place of an antibody.

In a preferred embodiment, the efficacy of anti-B cell antibodies for therapeutic use in autoimmune and/or immune dysfunction diseases is predicted by trogocytosis-mediated decrease in the levels of B-cell antigens on the cell surface, particularly that of CD19. Efficacy of anti-B-cell antibodies, such as anti-CD22 antibodies, for therapeutic use in specific autoimmune and/or immune dysfunction diseases may be predicted by measuring the extent of trogocytosis of cell surface markers, such as CD19 in B cells. The method may involve obtaining a sample of B cells from an individual with autoimmune or immune dysfunction disease, exposing the B cells to an anti-B cell (particularly anti-CD22) antibody, measuring the levels of CD19 in the B cells, and predicting the efficacy of the anti-B cell antibody for disease therapy. Alternatively, the method may involve administering the antibody to a subject and monitoring the level of trogocytosis and/or antigen shaving. In other alternative embodiments, the effect of anti-B cell antibody on inducing trogocytosis of CD19 may be used to predict the susceptibility of the diseased cell to antibody therapy and/or the prognosis of the individual with the disease. In still other embodiments, use of additional predictive factors such as FcγR polymorphisms may be incorporated into the method. The skilled artisan will realize that the same compositions and methods may be of use to provide a prognosis of autoimmune or immune dysfunction disease progression and/or to select an optimum dosage of anti-B cell antibody to administer to a patient with autoimmune and/or immune dysfunction diseases, including but not limited to systemic lupus erythematosus and ANCA-associated vasculitides.

Exemplary autoimmune or immune dysfunction diseases include acute immune thrombocytopenia, chronic immune thrombocytopenia, dermatomyositis, Sydenham's chorea, myasthenia gravis, systemic lupus erythematosus, lupus nephritis, rheumatic fever, polyglandular syndromes, bullous pemphigoid, pemphigus vulgaris, diabetes mellitus (e.g., juvenile diabetes), Henoch-Schonlein purpura, post-streptococcal nephritis, erythema nodosum, Takayasu's arteritis, ANCA-associated vasculitides, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjögren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, fibrosing alveolitis, graft-versus-host disease (GVHD), organ transplant rejection, sepsis, septicemia and inflammation.

In another embodiment, trogocytosis and/or antigen shaving may be utilized to select an optimal dosage of anti-B cell antibody, such as anti-CD22 antibody, to be administered to a subject with a malignancy, preferably a B-cell malignancy, such as non-Hodgkin's lymphoma, B-cell acute and chronic lymphoid leukemias, Burkitt lymphoma, Hodgkin's lymphoma, hairy cell leukemia, multiple myeloma and Waldenstrom's macroglobulinemia. Either in vitro or in vivo analysis may be performed. For example, a sample of whole blood or PBMCs may be obtained from a patient with a B-cell malignancy and incubated with different concentrations of anti-B cell antibody, such as anti-CD22 antibody. Dose-response curves may be constructed based on evidence of trogocytosis and/or antigen shaving from B cells. For example, relative cell surface expression levels of CD19, CD20, CD21, CD22, CD79b, CD44, CD62L and β7-integrin may be determined by standard assays, such as flow cytometry using fluorescence labeled antibodies. Depending on the disease to be treated, the optimum concentration of antibody to administer to the patient may be selected to either maximize or minimize trogocytosis and/or antigen shaving. The skilled artisan will realize that, for example, selection of an optimal dosage of anti-B-cell antibody to administer may preferably involve monitoring of relative cell surface expression of CD19, CD20, CD21, CD22, CD79b, CD44, CD62L and/or β7-integrin. However, the method is not limiting and monitoring of surrogate antigens or combinations of antigens may provide a preferred result. The skilled artisan will realize that the same methods and compositions may be used to determine the efficacy of an anti-B cell bispecific antibody against a B-cell malignancy, the prognosis of a B-cell malignancy, and/or the susceptibility of a malignant B cell to anti-B cell bispecific antibody.

Antibodies against B-cell surface proteins, such as CD19, CD20, CD21, CD22, CD79b, CD44, CD62L and β7-integrin, are known in the art and any such known antibody might be used in the claimed compositions and methods. An exemplary anti-CD20 antibody is hA20 (veltuzumab), disclosed for example in U.S. Pat. No. 7,251,164, the Examples section of which is incorporated herein by reference. Other known anti-CD20 antibodies of potential use include, but are not limited to, rituximab (Genentech, South San Francisco, Calif.), GA101 (obinutuzumab; R05072759, Roche, Basle, Switzerland), ofatumumab (GlaxoSmithKline, London, England), ocrelizumab (Roche, Nutley, N.J.), AME-133v (ocaratuzumab, MENTRIK Biotech, Dallas, Tex.), ibritumomab (Spectrum Pharmaceuticals, Irvine, Calif.) and PRO131921 (Genentech, South San Francisco, Calif.). An exemplary anti-CD19 antibody is hA19, disclosed for example in U.S. Pat. No. 7,109,304, the Examples section of which is incorporated herein by reference. Other known anti-CD19 antibodies of potential use include, but are not limited to, XmAb5574 (Xencor, Monrovia, Calif.), 5F3 (OriGene, Rockville, Md.), 4G7 (Pierce, Rockford, Ill.), 2E2 (Pierce, Rockford, Ill.), 1G9 (Pierce, Rockford, Ill.), LT19 (Santa Cruz Biotechnology, Santa Cruz, Calif.) and HD37 (Santa Cruz Biotechnology, Santa Cruz, Calif.). An exemplary anti-CD22 antibody is hLL2 (epratuzumab), disclosed for example in U.S. Pat. No. 7,074,403, the Examples section of which is incorporated herein by reference. Other known anti-CD22 antibodies of potential use include, but are not limited to, inotuzumab (Pfizer, Groton, Conn.), CAT-3888 (Cambridge Antibody Technology Group, Cambridge, England), CAT-8015 (Cambridge Antibody Technology Group, Cambridge, England), HB22.7 (Duke University, Durham, N.C.) and RFB4 (e.g., Invitrogen, Grand Island, N.Y.; Santa Cruz Biotechnology, Santa Cruz, Calif.). Exemplary anti-CD21 antibodies of potential use include, but are not limited to, LS-B7297 (LSBio, Seattle, Wash.), HB5 (eBioscience, San Diego, Calif.), A-3 (Santa Cruz Biotechnology, Santa Cruz, Calif.), D-19 (Santa Cruz Biotechnology, Santa Cruz, Calif.), Bly4 (Santa Cruz Biotechnology, Santa Cruz, Calif.), 1F8 (Abcam, Cambridge, Mass.) and Bu32 (BioLegend, San Diego, Calif.). Exemplary anti-CD79b antibodies of potential use include, but are not limited to, B29 (LSBio, Seattle, Wash.), 3A2-2E7 (LSBio, Seattle, Seattle, Wash.), CD3-1 (eBioscience, San Diego, Calif.) and SN8 (Santa Cruz Biotechnology, Santa Cruz, Calif.). Many such antibodies are publicly known and/or commercially available and any such known antibody may be utilized.

An antibody of use may be chimeric, humanized or human. The use of chimeric antibodies is preferred to the parent murine antibodies because they possess human antibody constant region sequences and therefore do not elicit as strong a human anti-mouse antibody (HAMA) response as murine antibodies. The use of humanized antibodies is even more preferred, in order to further reduce the possibility of inducing a HAMA reaction. Techniques for humanization of murine antibodies by replacing murine framework and constant region sequences with corresponding human antibody framework and constant region sequences are well known in the art and have been applied to numerous murine anti-cancer antibodies. Antibody humanization may also involve the substitution of one or more human framework amino acid residues with the corresponding residues from the parent murine framework region sequences. As discussed below, techniques for production of human antibodies are also well known.

The antibody may also be multivalent, or multivalent and multispecific. The antibody may include human constant regions of IgG1, IgG2, IgG3, or IgG4.

In certain embodiments, one or more anti-B-cell antibodies may be administered to a patient as part of a combination of antibodies or as a bispecific antibody. The antibodies may bind to different epitopes of the same antigen or to different antigens. Preferably, the antigens are selected from the group consisting of BCL-1, BCL-2, BCL-6, CD1a, CD2, CD3, CD4, CD5, CD7, CD8, CD10, CD11b, CD11c, CD13, CD14, CD15, CD16, CD19, CD20, CD21, CD22, CD23, CD25, CD33, CD34, CD38, CD40, CD40L, CD41a, CD43, CD45, CD55, CD56, CCD57, CD59, CD64, CD71, CD79a, CD79b, CD117, CD138, FMC-7 and HLA-DR. However, antibodies against other antigens of use for therapy of cancer, autoimmune diseases or immune dysfunction diseases are known in the art, as discussed below, and antibodies against any such disease-associated antigen known in the art may be utilized.

In more preferred embodiments, the allotype of the antibody may be selected to minimize host immunogenic response to the administered antibody, as discussed in more detail below. A preferred allotype is a non-G1m1 allotype (nG1m1), such as G1m3, G1m-3,1, G1m-3,2 or G1m-3,1,2. The non-G1m1 allotype is preferred for decreased antibody immunoreactivity. Surprisingly, repeated subcutaneous administration of concentrated nG1m1 antibody was not found to induce significant immune response, despite the enhanced immunogenicity of subcutaneous administration.

### BRIEF DESCRIPTION OF THE DRAWINGS

The following drawings are provided to illustrate preferred embodiments of the invention. However, the claimed subject matter is in no way limited by the illustrative embodiments disclosed in the drawings.

FIG. 1. Epratuzumab-induced reduction of select surface antigens on normal B cells. Fresh PBMCs isolated from the blood of healthy donors were treated overnight with 10 μg/mL epratuzumab or a non-binding isotype control mAb (hMN-14) and the relative surface levels of selected proteins on B cells were measured by flow cytometry. The effect of epratuzumab on 13 different B cell antigens was surveyed. The number of donors evaluated for each specific antigen is indicated in parentheses. The % mean fluorescence intensity of the isotype control treatment is shown. Error bars, Std. Dev.

FIG. 2. Reduction of CD19 on CD27+ and CD27⁻ B cells from three healthy donors (N1, N2 and N3). The % mean fluorescence intensity of the isotype control treatment is shown. Error bars, Std. Dev.

FIG. 3. Example of the reduction of CD19, CD22, CD21 and CD79b on CD27⁺ and CD27⁻ B cells from a healthy donor. The % mean fluorescence intensity of the isotype control treatment is shown. Error bars, Std. Dev.

FIG. 4. Comparison of the reduction of CD19 and CD21 on B cells following 2 h (N=5 donors) vs. overnight treatment (N=16 donors) with 10 μg/mL epratuzumab or isotype control (hMN-14). The % mean fluorescence intensity of the isotype control treatment is shown. Error bars, Std. Dev.

FIG. 5A. Histogram showing CD22 levels on B cells gated from PBMCs of healthy donors following overnight treatment with 10 μg/mL epratuzumab, hA19 (anti-CD19) or isotype control (hMN-14).

FIG. 5B. Histogram showing CD21 levels on B cells gated from PBMCs of healthy donors following overnight treatment with 10 μg/mL epratuzumab, hA19 (anti-CD19) or isotype control (hMN-14).

FIG. 6. Fresh PBMCs isolated from healthy donors were treated overnight with epratuzumab, veltuzumab or rituximab. The relative B cell count (B cells) and levels of CD19, CD22, CD21 and CD79b following treatment is shown as the % mean fluorescence intensity of the isotype control (hMN-14) treatment at the same protein concentration. Error bars, Std. Dev.

FIG. 7. Fresh PBMCs isolated from healthy donors were treated overnight with 10 μg/mL epratuzumab, 1 mg/mL epratuzumab or 10 μg/mL epratuzumab plus 1 mg/mL hMN-14. The B cell surface levels of CD19, CD21, CD22 and CD79b are shown as the % mean fluorescence intensity of the isotype control (hMN-14) treatment at the same protein concentration. Error bars, Std. Dev.

FIG. 8. PBMCs from two normal donors (N13 and N14) were treated overnight with epratuzumab or hMN-14 at varied concentrations (1 ng/mL-10 mg/mL). The B cell surface levels of CD19, CD21, CD22 and CD79b are shown as the % mean fluorescence intensity of the isotype control (hMN-14) treatment at the same protein concentration except for the 10 mg/mL epratuzumab, which was derived using 1 mg/mL hMN-14 as control. Error bars, Std. Dev.

FIG. 9. PBMCs were treated with whole IgG or an F(ab′)₂ fragment of epratuzumab at 10 μg/mL. The % mean fluorescence intensity of the isotype control (hMN-14) treatment at the same protein concentration is shown. Error bars, Std. Dev.

FIG. 10. Daudi human Burkitt lymphoma cells (1×10⁵ cells) were treated overnight with 10 μg/mL epratuzumab or an isotype control mAb (hMN-14) in the presence, or absence, of PBMCs (1×10⁶). The plot is shown as the % mean fluorescence intensity of the isotype control treatment. Error bars, Std. Dev.

FIG. 11. Raji human Burkitt lymphoma cells (1×10⁵ cells) were treated overnight with 10 μg/mL epratuzumab or an isotype control mAb (hMN-14) in the presence, or absence, of PBMCs (1×10⁶) or goat-anti-human IgG (20 μg/mL) as a crosslinking second antibody. The plot is shown as the % mean fluorescence intensity of the isotype control treatment. Error bars, Std. Dev.

FIG. 12. Gating of monocytes and T cells with anti-CD3 and anti-CD14 from PBMCs (top), T cell-depleted PBMCs (middle) and monocyte-depleted PBMCs (bottom).

FIG. 13. Epratuzumab-induced reduction of CD19 and CD22 with monocytes. Daudi cells (1×10⁵) were mixed with effector cells (1×10⁶) comprising PBMCs, T cell depleted-PBMCs or monocyte-depleted PBMCs, which were each derived from the same donor. The cell mixtures were incubated overnight with 10 μg/mL epratuzumab or an isotype control mAb (hMN-14). The level of CD19 and CD22 on the surface of Daudi (A) and the intrinsic B cells (B) were measured by flow cytometry and plotted as the % mean fluorescence intensity of the isotype control treatment.

FIG. 14. Purified T cells do not participate in epratuzumab-induced trogocytosis. Daudi cells (1×10⁵) were mixed with 1×10⁶ PBMCs or purified T cells, or without effector cells and treated overnight with 10 μg/mL epratuzumab or an isotype control mAb (hMN-14). The levels of CD19, CD21, CD22 and CD79b on the surface of Daudi was measured by flow cytometry and plotted as the % mean fluorescence intensity of the isotype control treatment.

FIG. 15. Gating of monocytes with anti-CD3 and anti-CD14 from PBMCs (top), monocyte-depleted PBMCs (middle) and purified monocytes (bottom).

FIG. 16. Daudi cells (1×10⁵) were mixed with PBMCs (1×10⁶), monocyte-depleted PBMCs (1×10⁶) or purified monocytes (5×10⁵), which were each derived from the same donor. The cell mixtures were incubated overnight with 10 μg/mL epratuzumab or an isotype control mAb (hMN-14). The level of CD19 and CD22 on the surface of Daudi were measured by flow and plotted as the % mean fluorescence intensity of the isotype control treatment.

FIG. 17. Gating of monocytes from PBMCs. The monocyte gate (top) was further separated into CD14++ and CD14+CD16+ sub-populations (bottom).

FIG. 18. (Top left) Gating by scattering from a mixture of purified monocytes and Daudi. (Top right) The Daudi cells were further identified as CD19+CD22+ cells in the Daudi gate. (Bottom) The monocyte gate was further separated into CD14++ and CD14+CD16+ sub-populations.

FIG. 19. Epratuzumab-induced trogocytosis with monocytes. Daudi cells were mixed with purified monocytes 1:1 and treated for 1 h with epratuzumab (black dots) or hMN-14 (white dots) before analysis by flow cytometry. The monocyte gate determined by forward vs. side scattering was further gated with anti-CD 14.

FIG. 20A. Daudi cells were mixed with purified monocytes 1:1 and treated for 1 h with epratuzumab (black dots) or hMN-14 (white dots) before analysis by flow cytometry. The monocyte gate determined by forward vs. side scattering was further separated into CD14⁺⁺ monocyte populations, which were each evaluated for CD19 and CD22 levels.

FIG. 20B. Daudi cells were mixed with purified monocytes 1:1 and treated for 1 h with epratuzumab (black dots) or hMN-14 (white dots) before analysis by flow cytometry. The monocyte gate determined by forward vs. side scattering was further separated into CD14⁺CD16⁺ monocyte populations, which were each evaluated for CD19 and CD22 levels.

FIG. 21. The Daudi cells (CD19⁺ cells in the Daudi gate) were analyzed for CD19 and CD22 levels following a 1-hour epratuzumab treatment with PBMCs, purified monocytes or monocyte-depleted PBMCs. The level of CD19 and CD22 on the surface of Daudi were measured by flow and plotted as the % mean fluorescence intensity of the isotype control treatment.

FIG. 22. (Top) Gating by scattering from a mixture of PBMCs and Daudi. (Bottom) The lymphocyte gate was further separated with CD14 and CD16 staining to identify NK cells.

FIG. 23A. Daudi cells were mixed with PBMCs 1:5 and treated for 1 h with epratuzumab (black dots) or hMN-14 (white dots) before analysis by flow cytometry. The NK cells were identified as CD14⁻CD16⁺ cells in the lymphocyte gate, which were evaluated for the levels of CD19 and CD22.

FIG. 23B. Daudi cells were mixed with monocyte-depleted PBMCs 1:5 and treated for 1 h with epratuzumab (black dots) or hMN-14 (white dots) before analysis by flow cytometry. The NK cells were identified as CD14⁻CD16⁺ cells in the lymphocyte gate, which were evaluated for the levels of CD19 and CD22.

FIG. 24. Gating of granulocytes mixed with Daudi first by forward vs. side scatter (Top) followed by anti-CD16 staining.

FIG. 25. Daudi cells were mixed with purified granulocytes 1:2 and treated for 1 h with epratuzumab (black dots) or hMN-14 (white dots) before analysis by flow cytometry. The granulocyte gate was further refined for CD16⁺ cells and evaluated for CD19 (A and B), CD22 (A) and CD79b (B) levels.

FIG. 26. Daudi cells were mixed with purified granulocytes 1:2 and treated for 1 h with epratuzumab or hMN-14 before analysis by flow cytometry. The Daudi cells (CD19⁺ cells in the Daudi gate) were analyzed for CD19, CD22 and CD79b levels and graphed as the % mean fluorescence intensity of the isotype control treatment.

FIG. 27. PBMCs were isolated from blood specimens of three naive SLE patients and treated overnight with 10 μg/mL epratuzumab or hMN-14. The relative levels of CD19, CD22, CD21 and CD79b on B cells post-treatment were measured by flow cytometry and graphed as the % mean fluorescence intensity of the isotype control treatment.

FIG. 28. PBMCs were isolated from blood specimens of three naive SLE patients and treated overnight with 10 μg/mL epratuzumab or hMN-14. B cells were gated further into CD27⁺ and CD27⁻ populations before analysis. The relative levels of CD19 and CD22 on the B cell sub-populations post-treatment were measured by flow cytometry and graphed as the % mean fluorescence intensity of the isotype control treatment.

FIG. 29. PBMCs were isolated from blood specimens of naive SLE patients and treated overnight with 10 μg/mL epratuzumab, an F(ab′)₂ of epratuzumab or hMN-14. B cells were gated further into CD27⁺ and CD27⁻ populations before analysis. The figure shows an example from one naive SLE patient. The relative levels of CD19, CD22, CD21 and CD79b on the B cell sub-populations post-treatment were measured by flow cytometry and graphed as the % mean fluorescence intensity of untreated PBMCs.

FIG. 30. The MFI levels of CD22 (A), CD19 (B), CD21 (C) and CD79b (D) were measured by flow cytometry on B cells gated from PBMCs that were isolated from four SLE patients who had yet to receive any treatment (naïve), five patients on active immunotherapy with epratuzumab and two patients on immunotherapy with BENLYSTA®. Each point represents an individual patient sample.

FIG. 31. Epratuzumab induces reduction of select surface antigens on normal B cells. PBMCs obtained from healthy donors were incubated overnight (16-24 h) with 10 μg/mL of either epratuzumab or an isotype control mAb (labetuzumab, anti-CEACAM5) and the relative levels of 13 different B-cell antigens were analyzed by flow cytometry. Based on PBMCs from 19 healthy donors assessed in various experiments, epratuzumab significantly reduced the levels of BCR modulating proteins, CD22, CD19, CD21 and CD79b; and also adhesion molecules CD44, CD62L and b7 integrin. The number of donors evaluated for each specific antigen is indicated in parentheses. Notably, CD27-naïve B cells were more responsive to epratuzumab compared to CD27+ memory B cells, as shown for the reduction of CD19 with PBMCs from 3 different healthy donors

FIG. 32. Trogocytosis mediated by Cₖ and C_{H}3-based bsAbs. PBMCs were incubated overnight with 10 μg/mL 22*-(20)-(20), 22-(20)-(20), veltuzumab, epratuzumab or labetuzumab (control), prior to measurement of surface CD19, CD22 and CD21 by flow cytometry. Results are shown as the % MFI of the control treatment. Error bars, Std. Dev.

FIG. 33A. B-cell depletion. Unless indicated otherwise, freshly isolated PBMCs were incubated for two days with 22*-(20)-(20) (red) or rituximab (blue) prior to counting the viable B cells, which were identified as 7-AAD⁻ cells in the lymphocyte gated that were either CD19⁺ or CD79b⁺. Sampling was normalized using counting beads, which were added to each sample before processing for flow cytometry. The relative viable B cell count is expressed as % Control, which was derived by dividing the specific B cell count by that measured following treatment with the control mAb (labetuzumab). Maximal B-cell depletion at 140 nM with PBMCs from 5 unique donors.

FIG. 33B. B-cell depletion. Unless indicated otherwise, freshly isolated PBMCs were incubated for two days with 22*-(20)-(20) (red) or rituximab (blue) prior to counting the viable B cells, which were identified as 7-AAD⁻ cells in the lymphocyte gated that were either CD19⁺ or CD79b⁺. Sampling was normalized using counting beads, which were added to each sample before processing for flow cytometry. The relative viable B cell count is expressed as % Control, which was derived by dividing the specific B cell count by that measured following treatment with the control mAb (labetuzumab). B-cell depletion at 24 h (square symbol, dashed lines) and 48 h (round symbol, solid line) with antibody titrations using PBMCs from Donor 4.

FIG. 33C. B-cell depletion. Unless indicated otherwise, freshly isolated PBMCs were incubated for two days with 22*-(20)-(20) (red) or rituximab (blue) prior to counting the viable B cells, which were identified as 7-AAD⁻ cells in the lymphocyte gated that were either CD19⁺ or CD79b⁺. Sampling was normalized using counting beads, which were added to each sample before processing for flow cytometry. The relative viable B cell count is expressed as % Control, which was derived by dividing the specific B cell count by that measured following treatment with the control mAb (labetuzumab). Daudi Burkitt lymphoma cells were spiked in PBMCs from Donor 3 and treated with titrations of the antibodies. Daudi (round symbol, dashed line) and normal B cells (square symbol, solid line) were separated by forward scattering and counted independently.

FIG. 33D. B-cell depletion. Unless indicated otherwise, freshly isolated PBMCs were incubated for two days with 22*-(20)-(20) (red) or rituximab (blue) prior to counting the viable B cells, which were identified as 7-AAD⁻ cells in the lymphocyte gated that were either CD19⁺ or CD79b⁺. Sampling was normalized using counting beads, which were added to each sample before processing for flow cytometry. The relative viable B cell count is expressed as % Control, which was derived by dividing the specific B cell count by that measured following treatment with the control mAb (labetuzumab). 22*-(20)-(20) (left, red) or rituximab (right, blue) were incubated at 140 nM with NK-depleted (ANK) or intact PBMCs, which were alternatively treated with Fc-deleted fragments (AFc/PBMC) of each antibody. Donor 1, solid bar; Donor 2, hatched bar.

FIG. 34A. Effector functions of 22*-(20)-(20) (red) and rituximab (blue). ADCC was measured using PBMCs of Donor 1 mixed at a 50:1 ratio with Daudi cells after 4 h.

FIG. 34B. Effector functions of 22*-(20)-(20) (red) and rituximab (blue). CDC. Epratuzumab, black trace. Error bars, Std. Dev.

### DETAILED DESCRIPTION

### Definitions

The following definitions are provided to facilitate understanding of the disclosure herein. Where a term is not specifically defined, it is used in accordance with its plain and ordinary meaning.

As used herein, the terms “a”, “an” and “the” may refer to either the singular or plural, unless the context otherwise makes clear that only the singular is meant.

An “antibody” refers to a full-length (i.e., naturally occurring or formed by normal immunoglobulin gene fragment recombinatorial processes) immunoglobulin molecule (e.g., an IgG antibody) or an immunologically active (i.e., antigen-binding) portion of an immunoglobulin molecule, like an antibody fragment.

An “antibody fragment” is a portion of an antibody such as F(ab′)₂, F(ab)₂, Fab′, Fab, Fv, scFv, single domain antibodies (e.g., nanobody) and the like, including half-molecules of IgG4 (van der Neut Kolfschoten et al., 2007, Science 317:1554-1557). Regardless of structure, an antibody fragment binds with the same antigen that is recognized by the intact antibody. For example, an anti-CD22 antibody fragment binds with an epitope of CD22. The term “antibody fragment” also includes isolated fragments consisting of the variable regions, such as the “Fv” fragments consisting of the variable regions of the heavy and light chains, recombinant single chain polypeptide molecules in which light and heavy chain variable regions are connected by a peptide linker (“scFv proteins”), and minimal recognition units consisting of the amino acid residues that mimic the hypervariable region.

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains, including human framework region (FR) sequences. The constant domains of the antibody molecule are derived from those of a human antibody.

A “human antibody” is an antibody obtained from transgenic mice that have been genetically engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

A “therapeutic agent” is an atom, molecule, or compound that is useful in the treatment of a disease. Examples of therapeutic agents include but are not limited to antibodies, antibody fragments, drugs, cytokine or chemokine inhibitors, pro-apoptotic agents, tyrosine kinase inhibitors, toxins, enzymes, nucleases, hormones, immunomodulators, antisense oligonucleotides, siRNA, RNAi, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

An “immunoconjugate” is a conjugate of an antibody with an atom, molecule, or a higher-ordered structure (e.g., with a liposome), a therapeutic agent, or a diagnostic agent. A “naked antibody” is an antibody that is not conjugated to any other agent.

A “naked antibody” is generally an entire antibody that is not conjugated to a therapeutic agent. This is so because the Fc portion of the antibody molecule provides effector functions, such as complement fixation and ADCC (antibody dependent cell cytotoxicity) that set mechanisms into action that may result in cell lysis. However, it is possible that the Fc portion is not required for therapeutic function, with other mechanisms, such as apoptosis, coming into play. Naked antibodies include both polyclonal and monoclonal antibodies, as well as certain recombinant antibodies, such as chimeric, humanized or human antibodies.

As used herein, the term “antibody fusion protein” is a recombinantly produced antigen-binding molecule in which an antibody or antibody fragment is linked to another protein or peptide, such as the same or different antibody or antibody fragment or a DDD or AD peptide (of the DOCK-AND-LOCK™ complexes described below). The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity.

A “bispecific antibody” is an antibody that can bind simultaneously to two targets which are of different structure. Bispecific antibodies (bsAb) and bispecific antibody fragments (bsFab) may have at least one arm that specifically binds to, for example, a B cell, T cell, myeloid-, plasma-, and mast-cell antigen or epitope and at least one other arm that specifically binds to a targetable conjugate that bears a therapeutic or diagnostic agent. A variety of bispecific antibodies can be produced using molecular engineering. Included herein are bispecific antibodies that target a cancer-associated antigen and also an immunotherapeutic T cell, such as CD3-T cells.

The term “direct cytotoxicity” refers to the ability of an agent to inhibit the proliferation or induce the apoptosis of a cell grown in an optimized culture medium in which only the agent and the cell are present.

Preparation of Monoclonal Antibodies

The compositions, formulations and methods described herein may include monoclonal antibodies. Rodent monoclonal antibodies to specific antigens may be obtained by methods known to those skilled in the art. (See, e.g., Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991)). General techniques for cloning murine immunoglobulin variable domains have been disclosed, for example, by the publication of Orlandi et al., Proc. Nat'l Acad. Sci. USA 86: 3833 (1989).

Chimeric Antibodies

A chimeric antibody is a recombinant protein that contains the variable domains including the CDRs derived from one species of animal, such as a rodent antibody, while the remainder of the antibody molecule; i.e., the constant domains, is derived from a human antibody. Techniques for constructing chimeric antibodies are well known to those of skill in the art. As an example, Leung et al., Hybridoma 13:469 (1994), disclose how they produced an LL2 chimera by combining DNA sequences encoding the Vₖ and V_{H} domains of LL2 monoclonal antibody, an anti-CD22 antibody, with respective human and IgG₁ constant region domains. This publication also provides the nucleotide sequences of the LL2 light and heavy chain variable regions, Vₖ and V_{H}, respectively.

Humanized Antibodies

A chimeric monoclonal antibody can be humanized by replacing the sequences of the murine FR in the variable domains of the chimeric antibody with one or more different human FR. Specifically, mouse CDRs are transferred from heavy and light variable chains of the mouse immunoglobulin into the corresponding variable domains of a human antibody. As simply transferring mouse CDRs into human FRs often results in a reduction or even loss of antibody affinity, additional modification might be required in order to restore the original affinity of the murine antibody. This can be accomplished by the replacement of one or more some human residues in the FR regions with their murine counterparts to obtain an antibody that possesses good binding affinity to its epitope. (See, e.g., Tempest et al., Biotechnology 9:266 (1991) and Verhoeyen et al., Science 239: 1534 (1988)). Techniques for producing humanized antibodies are disclosed, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993).

Human Antibodies

A fully human antibody can be obtained from a transgenic non-human animal. (See, e.g., Mendez et al., Nature Genetics, 15: 146-156, 1997; U.S. Pat. No. 5,633,425.) Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ. γ and κ chain antibody repertoires and inserted into a phage display library (Id.). RNAs were converted to cDNAs and used to make Fab cDNA libraries using specific primers against the heavy and light chain immunoglobulin sequences (Marks et al., 1991, J. Mol. Biol. 222:581-97). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XenoMouse® (e.g., Green et al., 1999, J. Immunol. Methods 231:11-23, incorporated herein by reference) from Abgenix (Fremont, Calif.). In the XenoMouse® and similar animals, the mouse antibody genes have been inactivated and replaced by functional human antibody genes, while the remainder of the mouse immune system remains intact.

The XenoMouse® was transformed with germline-configured YACs (yeast artificial chromosomes) that contained portions of the human IgH and Ig kappa loci, including the majority of the variable region sequences, along accessory genes and regulatory sequences. The human variable region repertoire may be used to generate antibody producing B cells, which may be processed into hybridomas by known techniques. A XenoMouse® immunized with a target antigen will produce human antibodies by the normal immune response, which may be harvested and/or produced by standard techniques discussed above. A variety of strains of XenoMouse® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XenoMouse® system but may utilize any transgenic animal that has been genetically engineered to produce human antibodies.

Antibody Cloning and Production

Various techniques, such as production of chimeric or humanized antibodies, may involve procedures of antibody cloning and construction. The antigen-binding Vκ (variable light chain) and V_{H} (variable heavy chain) sequences for an antibody of interest may be obtained by a variety of molecular cloning procedures, such as RT-PCR, 5′-RACE, and cDNA library screening. The V genes of an antibody from a cell that expresses a murine antibody can be cloned by PCR amplification and sequenced. To confirm their authenticity, the cloned V_{L} and V_{H} genes can be expressed in cell culture as a chimeric Ab as described by Orlandi et al., (Proc. Natl. Acad. Sci., USA, 86: 3833 (1989)). Based on the V gene sequences, a humanized antibody can then be designed and constructed as described by Leung et al. (Mol. Immunol., 32: 1413 (1995)).

cDNA can be prepared from any known hybridoma line or transfected cell line producing a murine antibody by general molecular cloning techniques (Sambrook et al., Molecular Cloning, A laboratory manual, 2^{nd} Ed (1989)). The Vκ sequence for the antibody may be amplified using the primers VK1BACK and VK1FOR (Orlandi et al., 1989) or the extended primer set described by Leung et al. (BioTechniques, 15: 286 (1993)). The V_{H} sequences can be amplified using the primer pair VH1BACK/VH1FOR (Orlandi et al., 1989) or the primers annealing to the constant region of murine IgG described by Leung et al. (Hybridoma, 13:469 (1994)). Humanized V genes can be constructed by a combination of long oligonucleotide template syntheses and PCR amplification as described by Leung et al. (Mol. Immunol., 32: 1413 (1995)).

PCR products for Vκ can be subcloned into a staging vector, such as a pBR327-based staging vector, VKpBR, that contains an Ig promoter, a signal peptide sequence and convenient restriction sites. PCR products for V_{H} can be subcloned into a similar staging vector, such as the pBluescript-based VHpBS. Expression cassettes containing the Vκ and V_{H} sequences together with the promoter and signal peptide sequences can be excised from VKpBR and VHpBS and ligated into appropriate expression vectors, such as pKh and pG1g, respectively (Leung et al., Hybridoma, 13:469 (1994)). The expression vectors can be co-transfected into an appropriate cell and supernatant fluids monitored for production of a chimeric, humanized or human antibody. Alternatively, the Vκ and V_{H} expression cassettes can be excised and subcloned into a single expression vector, such as pdHL2, as described by Gillies et al. (J. Immunol. Methods 125:191 (1989) and also shown in Losman et al., Cancer, 80:2660 (1997)).

In an alternative embodiment, expression vectors may be transfected into host cells that have been pre-adapted for transfection, growth and expression in serum-free medium. Exemplary cell lines that may be used include the Sp/EEE, Sp/ESF and Sp/ESF-X cell lines (see, e.g., U.S. Pat. Nos. 7,531,327; 7,537,930 and 7,608,425; the Examples section of each of which is incorporated herein by reference). These exemplary cell lines are based on the Sp2/0 myeloma cell line, transfected with a mutant Bcl-EEE gene, exposed to methotrexate to amplify transfected gene sequences and pre-adapted to serum-free cell line for protein expression.

Antibody Allotypes

Immunogenicity of therapeutic antibodies is associated with increased risk of infusion reactions and decreased duration of therapeutic response (Baert et al., 2003, N Engl J Med 348:602-08). The extent to which therapeutic antibodies induce an immune response in the host may be determined in part by the allotype of the antibody (Stickler et al., 2011, Genes and Immunity 12:213-21). Antibody allotype is related to amino acid sequence variations at specific locations in the constant region sequences of the antibody. The allotypes of IgG antibodies containing a heavy chain γ-type constant region are designated as Gm allotypes (1976, J Immunol 117:1056-59).

For the common IgG1 human antibodies, the most prevalent allotype is G1m1 (Stickler et al., 2011, Genes and Immunity 12:213-21). However, the G1m3 allotype also occurs frequently in Caucasians (Id.). It has been reported that G1m1 antibodies contain allotypic sequences that tend to induce an immune response when administered to non-G1m1 (nG1m1) recipients, such as G1m3 patients (Id.). Non-G1m1 allotype antibodies are not as immunogenic when administered to G1m1 patients (Id.).

The human G1m1 allotype comprises the amino acids aspartic acid at Kabat position 356 and leucine at Kabat position 358 in the CH3 sequence of the heavy chain IgG1. The nG1m1 allotype comprises the amino acids glutamic acid at Kabat position 356 and methionine at Kabat position 358. Both G1m1 and nG1m1 allotypes comprise a glutamic acid residue at Kabat position 357 and the allotypes are sometimes referred to as DEL and EEM allotypes. A non-limiting example of the heavy chain constant region sequences for G1m1 and nG1m1 allotype antibodies is shown for the exemplary antibodies rituximab (SEQ ID NO:86) and veltuzumab (SEQ ID NO:85).

Veltuzumab heavy chain constant region sequence
(SEQ ID NO: 85)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRW QQGNVFSCSVMHEALHNHYTQKSLSLSPGK
Rituximab heavy chain constant region sequence
(SEQ ID NO: 86)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKAEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRW QQGNVFSCSVMHEALHNHYTQKSLSLSPGK

Jefferis and Lefranc (2009, mAbs 1:1-7) reviewed sequence variations characteristic of IgG allotypes and their effect on immunogenicity. They reported that the G1m3 allotype is characterized by an arginine residue at Kabat position 214, compared to a lysine residue at Kabat 214 in the G1m17 allotype. The nG1m1,2 allotype was characterized by glutamic acid at Kabat position 356, methionine at Kabat position 358 and alanine at Kabat position 431. The G1m1,2 allotype was characterized by aspartic acid at Kabat position 356, leucine at Kabat position 358 and glycine at Kabat position 431. In addition to heavy chain constant region sequence variants, Jefferis and Lefranc (2009) reported allotypic variants in the kappa light chain constant region, with the Km1 allotype characterized by valine at Kabat position 153 and leucine at Kabat position 191, the Km1,2 allotype by alanine at Kabat position 153 and leucine at Kabat position 191, and the Km3 allotype characterized by alanine at Kabat position 153 and valine at Kabat position 191.

With regard to therapeutic antibodies, veltuzumab and rituximab are, respectively, humanized and chimeric IgG1 antibodies against CD20, of use for therapy of a wide variety of hematological malignancies and/or autoimmune diseases. Table 1 compares the allotype sequences of rituximab vs. veltuzumab. As shown in Table 1, rituximab (G1m17,1) is a DEL allotype IgG1, with an additional sequence variation at Kabat position 214 (heavy chain CH1) of lysine in rituximab vs. arginine in veltuzumab. It has been reported that veltuzumab is less immunogenic in subjects than rituximab (see, e.g., Morchhauser et al., 2009, J Clin Oncol 27:3346-53; Goldenberg et al., 2009, Blood 113:1062-70; Robak & Robak, 2011, BioDrugs 25:13-25), an effect that has been attributed to the difference between humanized and chimeric antibodies. However, the difference in allotypes between the EEM and DEL allotypes likely also accounts for the lower immunogenicity of veltuzumab.

TABLE 1
Allotypes of Rituximab vs. Veltuzumab

| | | Heavy chain position and associated allotypes | | | | | |
|---|---|---|---|---|---|---|---|
| | Complete | 214 | | 356/358 | | 431 | |
| | allotype | (allotype) | | (allotype) | | (allotype) | |
| Rituximab | G1m17,1 | K | 17 | D/L | 1 | A | — |
| Veltuzumab | G1m3 | R | 3 | E/M | — | A | — |

In order to reduce the immunogenicity of therapeutic antibodies in individuals of nG1m1 genotype, it is desirable to select the allotype of the antibody to correspond to the G1m3 allotype, characterized by arginine at Kabat 214, and the nG1m1,2 null-allotype, characterized by glutamic acid at Kabat position 356, methionine at Kabat position 358 and alanine at Kabat position 431. Surprisingly, it was found that repeated subcutaneous administration of G1m3 antibodies over a long period of time did not result in a significant immune response. In alternative embodiments, the human IgG4 heavy chain in common with the G1m3 allotype has arginine at Kabat 214, glutamic acid at Kabat 356, methionine at Kabat 359 and alanine at Kabat 431. Since immunogenicity appears to relate at least in part to the residues at those locations, use of the human IgG4 heavy chain constant region sequence for therapeutic antibodies is also a preferred embodiment. Combinations of G1m3 IgG1 antibodies with IgG4 antibodies may also be of use for therapeutic administration.

Known Antibodies

In various embodiments, the claimed methods and compositions may utilize any of a variety of antibodies known in the art. For example, therapeutic use of anti-B cell antibodies, such as anti-CD22 antibodies, may be supplemented with one or more antibodies against other disease-associated antigens. Antibodies of use may be commercially obtained from a number of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206; 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880, the Examples section of each of which is incorporated herein by reference).

Antibodies of use may bind to various known antigens expressed in B cells or T cells, including but not limited to BCL-1, BCL-2, BCL-6, CD1a, CD2, CD3, CD4, CD5, CD7, CD8, CD10, CD11b, CD11c, CD13, CD14, CD15, CD16, CD19, CD20, CD21, CD22, CD23, CD25, CD33, CD34, CD38, CD40, CD40L, CD41a, CD43, CD45, CD55, CD56, CCD57, CD59, CD64, CD71, CD79a, CD79b, CD117, CD138, FMC-7 and HLA-DR.

Particular antibodies that may be of use within the scope of the claimed methods and compositions include, but are not limited to, LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD-1 receptor), nivolumab (anti-PD-1 receptor), ipilimumab (anti-CTLA-4), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM5), MN-15 or MN-3 (anti-CEACAM6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin) and trastuzumab (anti-ErbB2). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20050271671; 20060193865; 20060210475; 20070087001; the Examples section of each incorporated herein by reference.) Specific known antibodies of use include hPAM4 (U.S. Pat. No. 7,282,567), hA20 (U.S. Pat. No. 7,251,164), hA19 (U.S. Pat. No. 7,109,304), hIMMU-31 (U.S. Pat. No. 7,300,655), hLL1 (U.S. Pat. No. 7,312,318), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. patent application Ser. No. 12/772,645), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575) the text of each recited patent or application is incorporated herein by reference with respect to the Figures and Examples sections.

Other useful antigens that may be targeted using the described conjugates include carbonic anhydrase IX, alpha-fetoprotein (AFP), α-actinin-4, A3, antigen specific for A33 antibody, ART-4, B7, Ba 733, BAGE, BrE3-antigen, CA125, CAMEL, CAP-1, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, HIF-1a, colon-specific antigen-p (CSAp), CEACAM5, CEACAM6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, T101, SAGE, 5100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207).

A comprehensive analysis of suitable antigen (Cluster Designation, or CD) targets on hematopoietic malignant cells, as shown by flow cytometry and which can be a guide to selecting suitable antibodies for drug-conjugated immunotherapy, is Craig and Foon, Blood prepublished online Jan. 15, 2008; DOL 10.1182/blood-2007-11-120535.

The CD66 antigens consist of five different glycoproteins with similar structures, CD66a-e, encoded by the carcinoembryonic antigen (CEA) gene family members, BCG, CGM6, NCA, CGM1 and CEA, respectively. These CD66 antigens (e.g., CEACAM6) are expressed mainly in granulocytes, normal epithelial cells of the digestive tract and tumor cells of various tissues. Also included as suitable targets for cancers are cancer testis antigens, such as NY-ESO-1 (Theurillat et al., Int. J. Cancer 2007; 120(11):2411-7), as well as CD79a in myeloid leukemia (Kozlov et al., Cancer Genet. Cytogenet. 2005; 163(1):62-7) and also B-cell diseases, and CD79b for non-Hodgkin's lymphoma (Poison et al., Blood 110(2):616-623). A number of the aforementioned antigens are disclosed in U.S. Provisional Application Ser. No. 60/426,379, entitled “Use of Multi-specific, Non-covalent Complexes for Targeted Delivery of Therapeutics,” filed Nov. 15, 2002. Cancer stem cells, which are ascribed to be more therapy-resistant precursor malignant cell populations (Hill and Penis, J. Natl. Cancer Inst. 2007; 99:1435-40), have antigens that can be targeted in certain cancer types, such as CD133 in prostate cancer (Maitland et al., Ernst Schering Found. Sympos. Proc. 2006; 5:155-79), non-small-cell lung cancer (Donnenberg et al., J. Control Release 2007; 122(3):385-91), and glioblastoma (Beier et al., Cancer Res. 2007; 67(9):4010-5), and CD44 in colorectal cancer (Dalerba er al., Proc. Natl. Acad. Sci. USA 2007; 104(24)10158-63), pancreatic cancer (Li et al., Cancer Res. 2007; 67(3):1030-7), and in head and neck squamous cell carcinoma (Prince et al., Proc. Natl. Acad. Sci. USA 2007; 104(3)973-8). Another useful target for breast cancer therapy is the LIV-1 antigen described by Taylor et al. (Biochem. J. 2003; 375:51-9).

For multiple myeloma therapy, suitable targeting antibodies have been described against, for example, CD38 and CD138 (Stevenson, Mol Med 2006; 12(11-12):345-346; Tassone et al., Blood 2004; 104(12):3688-96), CD74 (Stein et al., ibid.), CS1 (Tai et al., Blood 2008; 112(4):1329-37, and CD40 (Tai et al., 2005; Cancer Res. 65(13):5898-5906).

Macrophage migration inhibitory factor (MIF) is an important regulator of innate and adaptive immunity and apoptosis. It has been reported that CD74 is the endogenous receptor for MIF (Leng et al., 2003, J Exp Med 197:1467-76). The therapeutic effect of antagonistic anti-CD74 antibodies on MIF-mediated intracellular pathways may be of use for treatment of a broad range of disease states, such as cancers of the bladder, prostate, breast, lung, colon and chronic lymphocytic leukemia (e.g., Meyer-Siegler et al., 2004, BMC Cancer 12:34; Shachar & Haran, 2011, Leuk Lymphoma 52:1446-54); autoimmune diseases such as rheumatoid arthritis and systemic lupus erythematosus (Morand & Leech, 2005, Front Biosci 10:12-22; Shachar & Haran, 2011, Leuk Lymphoma 52:1446-54); kidney diseases such as renal allograft rejection (Lan, 2008, Nephron Exp Nephrol. 109:e79-83); and numerous inflammatory diseases (Meyer-Siegler et al., 2009, Mediators Inflamm epub Mar. 22, 2009; Takahashi et al., 2009, Respir Res 10:33; Milatuzumab (hLL1) is an exemplary anti-CD74 antibody of therapeutic use for treatment of MIF-mediated diseases.

Anti-TNF-α antibodies are known in the art and may be of use to treat immune diseases, such as autoimmune disease, immune dysfunction (e.g., graft-versus-host disease, organ transplant rejection) or diabetes. Known antibodies against TNF-α include the human antibody CDP571 (Ofei et al., 2011, Diabetes 45:881-85); murine antibodies MTNFAI, M2TNFAI, M3TNFAI, M3TNFABI, M302B and M303 (Thermo Scientific, Rockford, Ill.); infliximab (Centocor, Malvern, Pa.); certolizumab pegol (UCB, Brussels, Belgium); and adalimumab (Abbott, Abbott Park, Ill.). These and many other known anti-TNF-α antibodies may be used in the claimed methods and compositions. Other antibodies of use for therapy of immune dysregulatory or autoimmune disease include, but are not limited to, anti-B-cell antibodies such as veltuzumab, epratuzumab, milatuzumab or hL243; tocilizumab (anti-IL-6 receptor); basiliximab (anti-CD25); daclizumab (anti-CD25); efalizumab (anti-CD11a); muromonab-CD3 (anti-CD3 receptor); anti-CD40L (UCB, Brussels, Belgium); natalizumab (anti-α4 integrin) and omalizumab (anti-IgE).

Checkpoint inhibitor antibodies have been used primarily in cancer therapy. Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response against tumor tissues. Exemplary checkpoint inhibitor antibodies against cytotoxic T-lymphocyte antigen 4 (CTLA4, also known as CD152), programmed cell death protein 1 (PD1, also known as CD279) and programmed cell death 1 ligand 1 (PD-L1, also known as CD274), may be used in combination with one or more other agents to enhance the effectiveness of immune response against disease cells, tissues or pathogens. Exemplary anti-PD1 antibodies include lambrolizumab (MK-3475, MERCK), nivolumab (BMS-936558, BRISTOL-MYERS SQUIBB), AMP-224 (MERCK), and pidilizumab (CT-011, CURETECH LTD.). Anti-PD1 antibodies are commercially available, for example from ABCAM® (AB137132), BIOLEGEND® (EH12.2H7, RMP1-14) and AFFYMETRIX EBIOSCIENCE (J105, J116, MIH4). Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Anti-PD-L1 antibodies are also commercially available, for example from AFFYMETRIX EBIOSCIENCE (MIH1). Exemplary anti-CTLA4 antibodies include ipilimumab (Bristol-Myers Squibb) and tremelimumab (PFIZER). Anti-PD1 antibodies are commercially available, for example from ABCAM® (AB134090), SINO BIOLOGICAL INC. (11159-H03H, 11159-H08H), and THERMO SCIENTIFIC PIERCE (PA5-29572, PA5-23967, PA5-26465, MA1-12205, MA1-35914). Ipilimumab has recently received FDA approval for treatment of metastatic melanoma (Wada et al., 2013, J Transl Med 11:89).

In another preferred embodiment, antibodies are used that internalize rapidly and are then re-expressed, processed and presented on cell surfaces, enabling continual uptake and accretion of circulating conjugate by the cell. An example of a most-preferred antibody/antigen pair is LL1, an anti-CD74 MAb (invariant chain, class II-specific chaperone, Ii) (see, e.g., U.S. Pat. Nos. 6,653,104; 7,312,318; the Examples section of each incorporated herein by reference). The CD74 antigen is highly expressed on B-cell lymphomas (including multiple myeloma) and leukemias, certain T-cell lymphomas, melanomas, colonic, lung, and renal cancers, glioblastomas, and certain other cancers (Ong et al., Immunology 98:296-302 (1999)). A review of the use of CD74 antibodies in cancer is contained in Stein et al., Clin Cancer Res. 2007 Sep. 15; 13(18 Pt 2):5556s-5563s, incorporated herein by reference.

The diseases that are preferably treated with anti-CD74 antibodies include, but are not limited to, non-Hodgkin's lymphoma, Hodgkin's disease, melanoma, lung, renal, colonic cancers, glioblastome multiforme, histiocytomas, myeloid leukemias, and multiple myeloma. Continual expression of the CD74 antigen for short periods of time on the surface of target cells, followed by internalization of the antigen, and re-expression of the antigen, enables the targeting LL1 antibody to be internalized along with any chemotherapeutic moiety it carries. This allows a high, and therapeutic, concentration of LL1-chemotherapeutic drug conjugate to be accumulated inside such cells. Internalized LL1-chemotherapeutic drug conjugates are cycled through lysosomes and endosomes, and the chemotherapeutic moiety is released in an active form within the target cells.

Other antibodies of use for therapy of immune dysregulatory or autoimmune disease include, but are not limited to, anti-B-cell antibodies such as veltuzumab, epratuzumab, milatuzumab or hL243; tocilizumab (anti-IL-6 receptor); basiliximab (anti-CD25); daclizumab (anti-CD25); efalizumab (anti-CD11a); muromonab-CD3 (anti-CD3 receptor); OKT3 (anti-CDR3); anti-CD40L (UCB, Brussels, Belgium); natalizumab (anti-α4 integrin) and omalizumab (anti-IgE). Antibodies of use to treat autoimmune/immune dysfunction disease may bind to exemplary antigens including, but not limited to, BCL-1, BCL-2, BCL-6, CD1a, CD2, CD3, CD4, CD5, CD7, CD8, CD10, CD11b, CD11c, CD13, CD14, CD15, CD16, CD19, CD20, CD21, CD22, CD23, CD25, CD33, CD34, CD38, CD40, CD40L, CD41a, CD43, CD45, CD55, TNF-alpha, interferon, IL-6 and HLA-DR. Antibodies that bind to these and other target antigens, discussed above, may be used to treat autoimmune or immune dysfunction diseases. Autoimmune diseases that may be treated with immunoconjugates may include acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, ANCA-associated vasculitides, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, bullous pemphigoid, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis or fibrosing alveolitis.

Antibody Fragments

Antibody fragments which recognize specific epitopes can be generated by known techniques. The antibody fragments are antigen binding portions of an antibody, such as F(ab)₂, Fab′, Fab, Fv, scFv and the like. Other antibody fragments include, but are not limited to: the F(ab′)₂ fragments which can be produced by pepsin digestion of the antibody molecule and the Fab′ fragments, which can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are disclosed in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991).

An antibody fragment can be prepared by known methods, for example, as disclosed by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

A single complementarity-determining region (CDR) is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. (See, e.g., Larrick et al., Methods: A Companion to Methods in Enzymology 2: 106 (1991); Courtenay-Luck, “Genetic Manipulation of Monoclonal Antibodies,” in MONOCLONAL ANTIBODIES: PRODUCTION, ENGINEERING AND CLINICAL APPLICATION, Ritter et al. (eds.), pages 166-179 (Cambridge University Press 1995); and Ward et al., “Genetic Manipulation and Expression of Antibodies,” in MONOCLONAL ANTIBODIES: PRINCIPLES AND APPLICATIONS, Birch et al., (eds.), pages 137-185 (Wiley-Liss, Inc. 1995).

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 2006, 43:1912-19; Tanha et al., J. Biol. Chem. 2001, 276:24774-780).

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797; Hwang and Foote, 2005, Methods 36:3-10; Clark, 2000, Immunol Today 21:397-402; J Immunol 1976 117:1056-60; Ellison et al., 1982, Nucl Acids Res 13:4071-79; Stickler et al., 2011, Genes and Immunity 12:213-21).

Multispecific and Multivalent Antibodies

Methods for producing bispecific antibodies include engineered recombinant antibodies which have additional cysteine residues so that they crosslink more strongly than the more common immunoglobulin isotypes. (See, e.g., FitzGerald et al, Protein Eng. 10(10):1221-1225, (1997)). Another approach is to engineer recombinant fusion proteins linking two or more different single-chain antibody or antibody fragment segments with the needed dual specificities. (See, e.g., Coloma et al., Nature Biotech. 15:159-163, (1997)). A variety of bispecific antibodies can be produced using molecular engineering. In one form, the bispecific antibody may consist of, for example, an scFv with a single binding site for one antigen and a Fab fragment with a single binding site for a second antigen. In another form, the bispecific antibody may consist of, for example, an IgG with two binding sites for one antigen and two scFv with two binding sites for a second antigen. In alternative embodiments, multispecific and/or multivalent antibodies may be produced as DOCK-AND-LOCK™ (DNL™) complexes as described below.

In certain embodiments, one or more anti-B-cell antibodies may be administered to a patient as part of a combination of antibodies. Bispecific antibodies are preferred to administration of combinations of separate antibodies, due to cost and convenience. However, where combinations of separate antibodies provide improved safety or efficacy, the combination may be utilized. The antibodies may bind to different epitopes of the same antigen or to different antigens. Preferably, the antigens are selected from the group consisting of BCL-1, BCL-2, BCL-6, CD1a, CD2, CD3, CD4, CD5, CD7, CD8, CD10, CD11b, CD11c, CD13, CD14, CD15, CD16, CD19, CD20, CD21, CD22, CD23, CD25, CD33, CD34, CD38, CD40, CD40L, CD41a, CD43, CD45, CD55, CD56, CCD57, CD59, CD64, CD71, CD79a, CD79b, CD117, CD138, FMC-7 and HLA-DR. However, antibodies against other antigens of use for therapy of cancer, autoimmune diseases or immune dysfunction diseases are known in the art, as discussed below, and antibodies against any such disease-associated antigen known in the art may be utilized.

DOCK-AND-LOCK™ (DNL™)

In preferred embodiments, a bivalent or multivalent antibody is formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264. Wong and Scott, Nat. Rev. Mol. Cell. Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Although the standard DNL™ complex comprises a trimer with two DDD-linked molecules attached to one AD-linked molecule, variations in complex structure allow the formation of dimers, trimers, tetramers, pentamers, hexamers and other multimers. In some embodiments, the DNL™ complex may comprise two or more antibodies, antibody fragments or fusion proteins which bind to the same antigenic determinant or to two or more different antigens. The DNL™ complex may also comprise one or more other effectors, such as proteins, peptides, immunomodulators, cytokines, interleukins, interferons, binding proteins, peptide ligands, carrier proteins, toxins, ribonucleases such as onconase, inhibitory oligonucleotides such as siRNA, antigens or xenoantigens, polymers such as PEG, enzymes, therapeutic agents, hormones, cytotoxic agents, anti-angiogenic agents, pro-apoptotic agents or any other molecule or aggregate.

PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RII), and each type has α and β isoforms (Scott, Pharmacol. Ther. 1991; 50:123). Thus, the four isoforms of PKA regulatory subunits are RIα, RIβ, RIIα and RIIβ. The R subunits have been isolated only as stable dimers and the dimerization domain has been shown to consist of the first 44 amino-terminal residues of RIIα or RIIβ (Newlon et al., Nat. Struct. Biol. 1999; 6:222). As discussed below, similar portions of the amino acid sequences of other regulatory subunits are involved in dimerization and docking, each located at or near the N-terminal end of the regulatory subunit. Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265;21561)

Since the first AKAP, microtubule-associated protein-2, was characterized in 1984 (Lohmann et al., Proc. Natl. Acad. Sci. USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell. Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIα are both located within the same N-terminal 44 amino acid sequence (Newlon et al., Nat. Struct. Biol. 1999; 6:222; Newlon et al., EMBO J. 2001; 20:1651), which is termed the DDD herein.

We have developed a platform technology to utilize the DDD of human PKA regulatory subunits and the AD of AKAP as an excellent pair of linker modules for docking any two entities, referred to hereafter as A and B, into a noncovalent complex, which could be further locked into a DNL™ complex through the introduction of cysteine residues into both the DDD and AD at strategic positions to facilitate the formation of disulfide bonds. The general methodology of the approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically. Using various combinations of linkers, adaptor modules and precursors, a wide variety of DNL™ constructs of different stoichiometry may be produced and used (see, e.g., U.S. Pat. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. Utilizing the fusion protein method of constructing AD and DDD conjugated effectors described in the Examples below, virtually any protein or peptide may be incorporated into a DNL™ construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

Structure-Function Relationships in AD and DDD Moieties

For different types of DNL™ constructs, different AD or DDD sequences may be utilized. Exemplary DDD and AD sequences are provided below.

DDD1
(SEQ ID NO: 1)
SHIQIPPGLTELLQGYTVEVLRQQPPDLVEFAVEYFTRLREARA
DDD2
(SEQ ID NO: 2)
CGHIQIPPGLTELLQGYTVEVLRQQPPDLVEFAVEYFTRLREARA
AD1
(SEQ ID NO: 3)
QIEYLAKQIVDNAIQQA
AD2
(SEQ ID NO: 4)
CGQIEYLAKQIVDNAIQQAGC

The skilled artisan will realize that DDD1 and DDD2 are based on the DDD sequence of the human RIIα isoform of protein kinase A. However, in alternative embodiments, the DDD and AD moieties may be based on the DDD sequence of the human RIα form of protein kinase A and a corresponding AKAP sequence, as exemplified in DDD3, DDD3C and AD3 below.

DDD3
(SEQ ID NO: 5)
SLRECELYVQKHNIQALLKDSIVQLCTARPERPMAFLREYFERLEKEEAK
DDD3C
(SEQ ID NO: 6)
MSCGGSLRECELYVQKHNIQALLKDSIVQLCTARPERPMAFLR
EYFERLEKEEAK
AD3
(SEQ ID NO: 7)
CGFEELAWKIAKMIWSDVFQQGC

In other alternative embodiments, other sequence variants of AD and/or DDD moieties may be utilized in construction of the DNL™ complexes. For example, there are only four variants of human PKA DDD sequences, corresponding to the DDD moieties of PKA RIα, RIIα, RIβ and RIIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 8)
SLRECELYVQKHNIQALLKDVSIVQLCTARPERPMAFLREYFEKLEKEEAK
PKA RIβ
(SEQ ID NO: 9)
SLKGCELYVQLHGIQQVLKDCIVHLCISKPERPMKFLREHFEKLEKEENRQ
ILA
PKA RIIα
(SEQ ID NO: 10)
SHIQIPPGLTELLQGYTVEVGQQPPDLVDFAVEYFTRLREARRQ
PKA RIIβ
(SEQ ID NO: 11)
SIEIPAGLTELLQGFTVEVLRHQPADLLEFALQHFTRLQQENER

The structure-function relationships of the AD and DDD domains have been the subject of investigation. (See, e.g., Burns-Hamuro et al., 2005, Protein Sci 14:2982-92; Carr et al., 2001, J Biol Chem 276:17332-38; Alto et al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24:397-408, the entire text of each of which is incorporated herein by reference.)

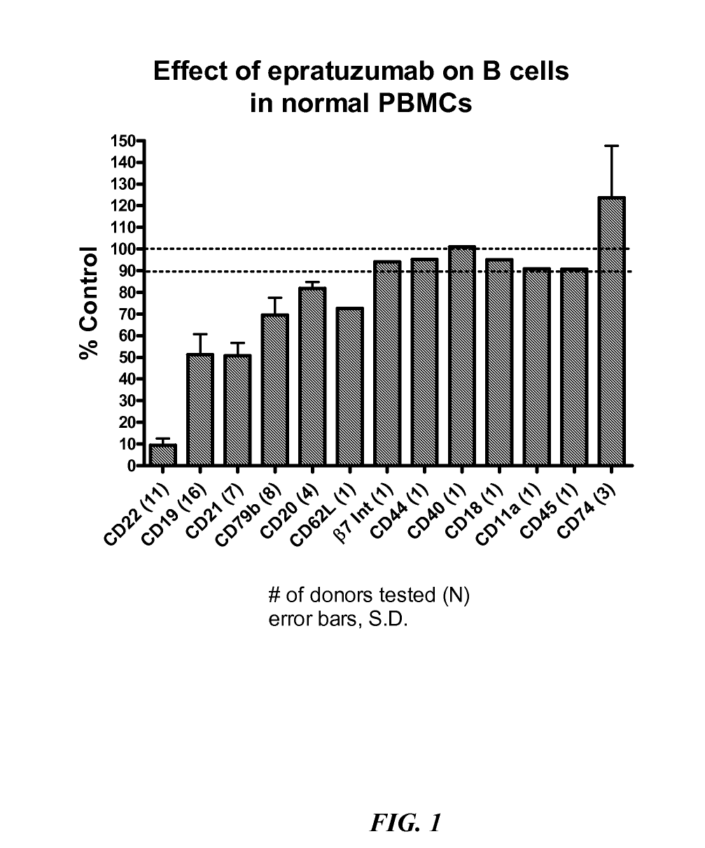

For example, Kinderman et al. (2006, Mol Cell 24:397-408) examined the crystal structure of the AD-DDD binding interaction and concluded that the human DDD sequence contained a number of conserved amino acid residues that were important in either dimer formation or AKAP binding, underlined in SEQ ID NO:1 below. (See FIG. 1 of Kinderman et al., 2006, incorporated herein by reference.) The skilled artisan will realize that in designing sequence variants of the DDD sequence, one would desirably avoid changing any of the underlined residues, while conservative amino acid substitutions might be made for residues that are less critical for dimerization and AKAP binding.

(SEQ ID NO: 1)
SHIQIPPGLTELLQGYTVEVLRQQPPDLVEFAVEYFTRLREARA

As discussed in more detail below, conservative amino acid substitutions have been characterized for each of the twenty common L-amino acids. Thus, based on the data of Kinderman (2006) and conservative amino acid substitutions, potential alternative DDD sequences based on SEQ ID NO:1 are shown in Table 2. In devising Table 2, only highly conservative amino acid substitutions were considered. For example, charged residues were only substituted for residues of the same charge, residues with small side chains were substituted with residues of similar size, hydroxyl side chains were only substituted with other hydroxyls, etc. Because of the unique effect of proline on amino acid secondary structure, no other residues were substituted for proline. A limited number of such potential alternative DDD moiety sequences are shown in SEQ ID NO:12 to SEQ ID NO:31 below. The skilled artisan will realize that an almost unlimited number of alternative species within the genus of DDD moieties can be constructed by standard techniques, for example using a commercial peptide synthesizer or well known site-directed mutagenesis techniques. The effect of the amino acid substitutions on AD moiety binding may also be readily determined by standard binding assays, for example as disclosed in Alto et al. (2003, Proc Natl Acad Sci USA 100:4445-50).

TABLE 2
Conservative Amino Acid Substitutions in DDD1 (SEQ ID NO: 1). Consensus
sequence disclosed as SEQ ID NO: 87.

| | | | | | | | | | | | | | | | | | | | | | |
|---|---|---|---|---|---|---|---|---|---|---|---|---|---|---|---|---|---|---|---|---|---|
| S | H | I | Q | I | P | P | G | L | T | E | L | L | Q | G | Y | T | V | E | V | L | R |
| T | K | | N | | | | A | | S | D | | | N | A | | S | | D | | | K |
| | R | | | | | | | | | | | | | | | | | | | | |
| Q | Q | P | P | D | L | V | E | F | A | V | E | Y | F | T | R | L | R | E | A | R | A |
| N | N | | | E | | | D | | L | | D | | | S | K | | K | D | L | K | L |
| | | | | | | | | | I | | | | | | | | | | I | I | |
| | | | | | | | | | V | | | | | | | | | | V | | V |

THIQIPPGLTELLQGYTVEVLRQQPPDLVEFAVEYFTRLREARA (SEQ ID NO: 12)
SKIQIPPGLTELLQGYTVEVLRQQPPDLVEFAVEYFTRLREARA (SEQ ID NO: 13)
SRIQIPPGLTELLQGYTVEVLRQQPPDLVEFAVEYFTRLREARA (SEQ ID NO: 14)
SHINIPPGLTELLQGYTVEVLRQQPPDLVEFAVEYFTRLREARA (SEQ ID NO: 15)
SHIQIPPALTELLQGYTVEVLRQQPPDLVEFAVEYFTRLREARA (SEQ ID NO: 16)
SHIQIPPGLSELLQGYTVEVLRQQPPDLVEFAVEYFTRLREARA (SEQ ID NO: 17)
SHIQIPPGLTDLLQGYTVEVLRQQPPDLVEFAVEYFTRLREARA (SEQ ID NO: 18)
SHIQIPPGLTELLNGYTVEVLRQQPPDLVEFAVEYFTRLREARA (SEQ ID NO: 19)
SHIQIPPGLTELLQAYTVEVLRQQPPDLVEFAVEYFTRLREARA (SEQ ID NO: 20)
SHIQIPPGLTELLQGYSVEVLRQQPPDLVEFAVEYFTRLREARA (SEQ ID NO: 21)
SHIQIPPGLTELLQGYTVDVLRQQPPDLVEFAVEYFTRLREARA (SEQ ID NO: 22)
SHIQIPPGLTELLQGYTVEVLKQQPPDLVEFAVEYFTRLREARA (SEQ ID NO: 23)
SHIQIPPGLTELLQGYTVEVLRNQPPDLVEFAVEYFTRLREARA (SEQ ID NO: 24)
SHIQIPPGLTELLQGYTVEVLRQNPPDLVEFAVEYFTRLREARA (SEQ ID NO: 25)
SHIQIPPGLTELLQGYTVEVLRQQPPELVEFAVEYFTRLREARA (SEQ ID NO: 26)
SHIQIPPGLTELLQGYTVEVLRQQPPDLVDFAVEYFTRLREARA (SEQ ID NO: 27)
SHIQIPPGLTELLQGYTVEVLRQQPPDLVEFLVEYFTRLREARA (SEQ ID NO: 28)
SHIQIPPGLTELLQGYTVEVLRQQPPDLVEFIVEYFTRLREARA (SEQ ID NO: 29)
SHIQIPPGLTELLQGYTVEVLRQQPPDLVEFVVEYFTRLREARA (SEQ ID NO: 30)
SHIQIPPGLTELLQGYTVEVLRQQPPDLVEFAVDYFTRLREARA (SEQ ID NO: 31)

Alto et al. (2003, Proc Natl Acad Sci USA 100:4445-50) performed a bioinformatic analysis of the AD sequence of various AKAP proteins to design an RII selective AD sequence called AKAP-IS (SEQ ID NO:3), with a binding constant for DDD of 0.4 nM. The AKAP-IS sequence was designed as a peptide antagonist of AKAP binding to PKA. Residues in the AKAP-IS sequence where substitutions tended to decrease binding to DDD are underlined in SEQ ID NO:3 below. The skilled artisan will realize that in designing sequence variants of the AD sequence, one would desirably avoid changing any of the underlined residues, while conservative amino acid substitutions might be made for residues that are less critical for DDD binding. Table 3 shows potential conservative amino acid substitutions in the sequence of AKAP-IS (AD1, SEQ ID NO:3), similar to that shown for DDD1 (SEQ ID NO:1) in Table 2 above.

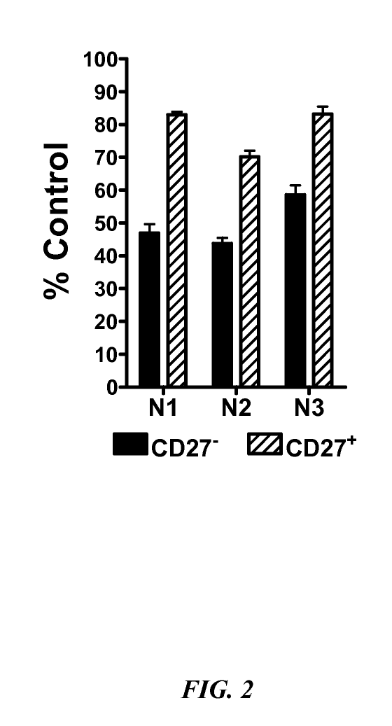

A limited number of such potential alternative AD moiety sequences are shown in SEQ ID NO:32 to SEQ ID NO:49 below. Again, a very large number of species within the genus of possible AD moiety sequences could be made, tested and used by the skilled artisan, based on the data of Alto et al. (2003). It is noted that FIG. 2 of Alto (2003) shows an even large number of potential amino acid substitutions that may be made, while retaining binding activity to DDD moieties, based on actual binding experiments.

AKAP-IS
(SEQ ID NO: 3)
QIEYLAKQIVDNAIQQA

TABLE 3
Conservative Amino Acid Substitutions in AD1 (SEQ ID NO: 3). Consensus
sequence disclosed as SEQ ID NO: 88.

| | | | | | | | | | | | | | | | | |
|---|---|---|---|---|---|---|---|---|---|---|---|---|---|---|---|---|
| Q | I | E | Y | L | A | K | Q | I | Y | D | N | A | I | Q | Q | A |
| N | L | D | F | I | | R | N | | E | Q | | | N | N | L | |
| | V | | T | V | | | | | | | | | | | | I |
| | | | S | | | | | | | | | | | | | V |

NIEYLAKQIVDNAIQQA (SEQ ID NO: 32)
QLEYLAKQIVDNAIQQA (SEQ ID NO: 33)
QVEYLAKQIVDNAIQQA (SEQ ID NO: 34)
QIDYLAKQIVDNAIQQA (SEQ ID NO: 35)
QIEFLAKQIVDNAIQQA (SEQ ID NO: 36)
QIETLAKQIVDNAIQQA (SEQ ID NO: 37)
QIESLAKQIVDNAIQQA (SEQ ID NO: 38)
QIEYIAKQIVDNAIQQA (SEQ ID NO: 39)
QIEYVAKQIVDNAIQQA (SEQ ID NO: 40)
QIEYLARQIVDNAIQQA (SEQ ID NO: 41)
QIEYLAKNIVDNAIQQA (SEQ ID NO: 42)
QIEYLAKQIVENAIQQA (SEQ ID NO: 43)
QIEYLAKQIVDQAIQQA (SEQ ID NO: 44)
QIEYLAKQIVDNAINQA (SEQ ID NO: 45)
QIEYLAKQIVDNAIQNA (SEQ ID NO: 46)
QIEYLAKQIVDNAIQQL (SEQ ID NO: 47)
QIEYLAKQIVDNAIQQI (SEQ ID NO: 48)
QIEYLAKQIVDNAIQQV (SEQ ID NO: 49)

Gold et al. (2006, Mol Cell 24:383-95) utilized crystallography and peptide screening to develop a SuperAKAP-IS sequence (SEQ ID NO:50), exhibiting a five order of magnitude higher selectivity for the RII isoform of PKA compared with the RI isoform. Underlined residues indicate the positions of amino acid substitutions, relative to the AKAP-IS sequence, which increased binding to the DDD moiety of RIIα. In this sequence, the N-terminal Q residue is numbered as residue number 4 and the C-terminal A residue is residue number 20. Residues where substitutions could be made to affect the affinity for RIIα were residues 8, 11, 15, 16, 18, 19 and 20 (Gold et al., 2006). It is contemplated that in certain alternative embodiments, the SuperAKAP-IS sequence may be substituted for the AKAP-IS AD moiety sequence to prepare DNL™ constructs. Other alternative sequences that might be substituted for the AKAP-IS AD sequence are shown in SEQ ID NO:51-53. Substitutions relative to the AKAP-IS sequence are underlined. It is anticipated that, as with the AD2 sequence shown in SEQ ID NO:4, the AD moiety may also include the additional N-terminal residues cysteine and glycine and C-terminal residues glycine and cysteine.

SuperAKAP-IS
(SEQ ID NO: 50)
QIEYVAKQIVDYAIHQA
Alternative AKAP sequences
(SEQ ID NO: 51)
QIEYKAKQIVDHAIHQA
(SEQ ID NO: 52)
QIEYHAKQIVDHAIHQA
(SEQ ID NO: 53)
QIEYVAKQIVDHAIHQA

FIG. 2 of Gold et al. disclosed additional DDD-binding sequences from a variety of AKAP proteins, shown below.

RII-Specific AKAPs
AKAP-KL
(SEQ ID NO: 54)
PLEYQAGLLVQNAIQQAI
AKAP79
(SEQ ID NO: 55)
LLIETASSLVKNAIQLSI
AKAP-Lbc
(SEQ ID NO: 56)
LIEEAASRIVDAVIEQVK
RI-Specific AKAPs
AKAPce
(SEQ ID NO: 57)
ALYQFADRFSELVISEAL
RIAD
(SEQ ID NO: 58)
LEQVANQLADQIIKEAT
PV38
(SEQ ID NO: 59)
FEELAWKIAKMIWSDVF
Dual-Specificity AKAPs
AKAP7
(SEQ ID NO: 60)
ELVRLSKRLVENAVLKAV
MAP2D
(SEQ ID NO: 61)
TAEEVSARIVQVVTAEAV
DAKAP1
(SEQ ID NO: 62)
QIKQAAFQLISQVILEAT
DAKAP2
(SEQ ID NO: 63)
LAWKIAKMIVSDVMQQ

Stokka et al. (2006, Biochem J 400:493-99) also developed peptide competitors of AKAP binding to PKA, shown in SEQ ID NO:64-66. The peptide antagonists were designated as Ht31 (SEQ ID NO:64), RIAD (SEQ ID NO:65) and PV-38 (SEQ ID NO:66). The Ht-31 peptide exhibited a greater affinity for the RH isoform of PKA, while the RIAD and PV-38 showed higher affinity for RI.

Ht31
(SEQ ID NO: 64)
DLIEEAASRIVDAVIEQVKAAGAY
RIAD
(SEQ ID NO: 65)
LEQYANQLADQIIKEATE
PV-38
(SEQ ID NO: 66)
FEELAWKIAKMIWSDVFQQC

Hundsrucker et al. (2006, Biochem J 396:297-306) developed still other peptide competitors for AKAP binding to PKA, with a binding constant as low as 0.4 nM to the DDD of the RII form of PKA. The sequences of various AKAP antagonistic peptides are provided in Table 1 of Hundsrucker et al., reproduced in Table 4 below. AKAPIS represents a synthetic RII subunit-binding peptide. All other peptides are derived from the RII-binding domains of the indicated AKAPs.

TABLE 4
AKAP Peptide sequences

| | Peptide Sequence |
|---|---|
| (SEQ ID NO: 3) | |
| AKAPIS | QIEYLAKQIVDNAIQQA |
| (SEQ ID NO: 67) | |
| AKAPIS-P | QIEYLAKQIPDNAIQQA |
| (SEQ ID NO: 68) | |
| Ht31 | KGADLIEEAASRIVDAVIEQVKAAG |
| (SEQ ID NO: 69) | |
| Ht31-P | KGADLIEEAASRIPDAPIEQVKAAG |
| (SEQ ID NO: 70) | |
| AKAP7δ-wt-pep | PEDAELVRLSKRLVENAVLKAVQQY |
| (SEQ ID NO: 71) | |
| AKAP7δ-L304T-pep | PEDAELVRTSKRLVENAVLKAVQQY |
| (SEQ ID NO: 72) | |
| AKAP7δ-L308D-pep | PEDAELVRLSKRDVENAVLKAVQQY |
| (SEQ ID NO: 73) | |
| AKAP7δ-P-pep | PEDAELVRLSKRLPENAVLKAVQQY |
| (SEQ ID NO: 74) | |
| AKAP7δ-PP-pep | PEDAELVRLSKRLPENAPLKAVQQY |
| (SEQ ID NO: 75) | |
| AKAP7δ-L314E-pep | PEDAELVRLSKRLVENAVEKAVQQY |
| (SEQ ID NO: 76) | |
| AKAP1-pep | EEGLDRNEEIKRAAFQIISQVISEA |
| (SEQ ID NO: 77) | |
| AKAP2-pep | LVDDPLEYQAGLLVQNAIQQAIAEQ |
| (SEQ ID NO: 78) | |
| AKAP5-pep | QYETLLIETASSLVKNAIQLSIEQL |
| (SEQ ID NO: 79) | |
| AKAP9-pep | LEKQYQEQLEEEVAKVIVSMSIAFA |
| (SEQ ID NO: 80) | |
| AKAP10-pep | NTDEAQEELAWKIAKMIVSDIMQQA |
| (SEQ ID NO: 81) | |
| AKAP11-pep | VNLDKKAVLAEKIVAEAIEKAEREL |
| (SEQ ID NO: 82) | |
| AKAP12-pep | NGILELETKSSKLVQNIIQTAVDQF |
| (SEQ ID NO: 83) | |
| AKAP14-pep | TQDKNYEDELTQVALALVEDVINYA |
| (SEQ ID NO: 84) | |
| Rab32-pep | ETSAKDNINIEEAARFLVEKILVNH |

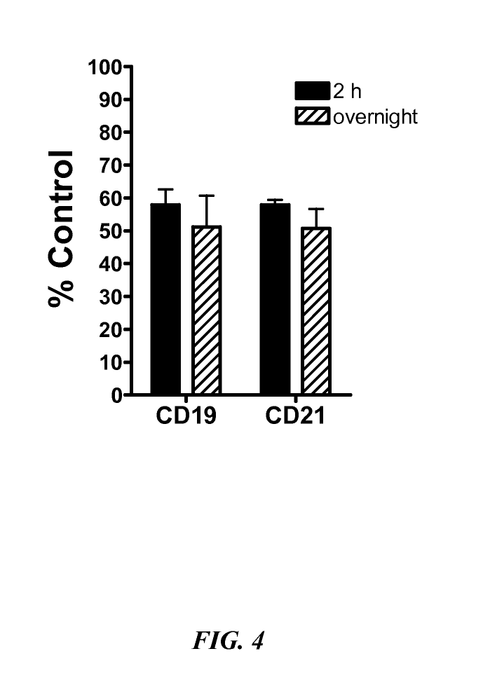

Residues that were highly conserved among the AD domains of different AKAP proteins are indicated below by underlining with reference to the AKAP IS sequence (SEQ ID NO:3). The residues are the same as observed by Alto et al. (2003), with the addition of the C-terminal alanine residue. (See FIG. 4 of Hundsrucker et al. (2006), incorporated herein by reference.) The sequences of peptide antagonists with particularly high affinities for the RII DDD sequence were those of AKAP-IS, AKAP7δ-wt-pep, AKAP7δ-L304T-pep and AKAP7δ-L308D-pep.

AKAP-IS
(SEQ ID NO: 3)
QIEYLAKQIVDNAIQQA

Can et al. (2001, J Biol Chem 276:17332-38) examined the degree of sequence homology between different AKAP-binding DDD sequences from human and non-human proteins and identified residues in the DDD sequences that appeared to be the most highly conserved among different DDD moieties. These are indicated below by underlining with reference to the human PKA RIIα DDD sequence of SEQ ID NO:1. Residues that were particularly conserved are further indicated by italics. The residues overlap with, but are not identical to those suggested by Kinderman et al. (2006) to be important for binding to AKAP proteins. The skilled artisan will realize that in designing sequence variants of DDD, it would be most preferred to avoid changing the most conserved residues (italicized), and it would be preferred to also avoid changing the conserved residues (underlined), while conservative amino acid substitutions may be considered for residues that are neither underlined nor italicized.

(SEQ ID NO: 1)
SHIQIPPGLTELLQGYTVEVLRQQPPDLVEFAVEYFTRLREARA

A modified set of conservative amino acid substitutions for the DDD1 (SEQ ID NO:1) sequence, based on the data of Carr et al. (2001) is shown in Table 5. Even with this reduced set of substituted sequences, there are over 65,000 possible alternative DDD moiety sequences that may be produced, tested and used by the skilled artisan without undue experimentation. The skilled artisan could readily derive such alternative DDD amino acid sequences as disclosed above for Table 2 and Table 3.

TABLE 5
Conservative Amino Acid Substitutions in DDD1 (SEQ ID
NO: 1). Consensus sequence disclosed as SEQ ID NO: 89.

| S | H | I | Q | [symbol] | [symbol] | P | [symbol] | [symbol] | T | E | [symbol] | [symbol] | Q | [symbol] | [symbol] | [symbol] | V | [symbol] | [symbol] | [symbol] | [symbol] |
|---|---|---|---|---|---|---|---|---|---|---|---|---|---|---|---|---|---|---|---|---|---|
| T | | | N | | | | | | S | | | | | | | | I | | | | |
| | | | | | | | | | | | | | | | | | L | | | | |
| | | | | | | | | | | | | | | | | | A | | | | |

| Q | [symbol] | [symbol] | P | [symbol] | [symbol] | V | E | [symbol] | [symbol] | V | E | [symbol] | [symbol] | T | R | [symbol] | R | E | A | [symbol] | A |
|---|---|---|---|---|---|---|---|---|---|---|---|---|---|---|---|---|---|---|---|---|---|
| N | | | | | | | | | | I | D | | | S | K | | K | | L | | L |
| | | | | | | | | | | L | | | | | | | | | I | | I |
| | | | | | | | | | | A | | | | | | | | | V | | V |

The skilled artisan will realize that these and other amino acid substitutions in the DDD or AD amino acid sequences may be utilized to produce alternative species within the genus of AD or DDD moieties, using techniques that are standard in the field and only routine experimentation.

Amino Acid Substitutions

In alternative embodiments, the disclosed methods and compositions may involve production and use of proteins or peptides with one or more substituted amino acid residues. For example, the DDD and/or AD sequences used to make DNL™ constructs may be modified as discussed above.

The skilled artisan will be aware that, in general, amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (O) glu, asn; Glu (E) gln, asp; Gly (G) ala; H is (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL website at rockefeller.edu) For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.). Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., H is, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

Affibodies and Fynomers

Certain alternative embodiments may utilize affibodies in place of antibodies. Affibodies are commercially available from Affibody AB (Solna, Sweden). Affibodies are small proteins that function as antibody mimetics and are of use in binding target molecules. Affibodies were developed by combinatorial engineering on an alpha helical protein scaffold (Nord et al., 1995, Protein Eng 8:601-8; Nord et al., 1997, Nat Biotechnol 15:772-77). The affibody design is based on a three helix bundle structure comprising the IgG binding domain of protein A (Nord et al., 1995; 1997). Affibodies with a wide range of binding affinities may be produced by randomization of thirteen amino acids involved in the Fc binding activity of the bacterial protein A (Nord et al., 1995; 1997). After randomization, the PCR amplified library was cloned into a phagemid vector for screening by phage display of the mutant proteins. The phage display library may be screened against any known antigen, using standard phage display screening techniques (e.g., Pasqualini and Ruoslahti, 1996, Nature 380:364-366; Pasqualini, 1999, Quart. J. Nucl. Med. 43:159-162), in order to identify one or more affibodies against the target antigen.

A ¹⁷⁷Lu-labeled affibody specific for HER2/neu has been demonstrated to target HER2-expressing xenografts in vivo (Tolmachev et al., 2007, Cancer Res 67:2773-82). Although renal toxicity due to accumulation of the low molecular weight radiolabeled compound was initially a problem, reversible binding to albumin reduced renal accumulation, enabling radionuclide-based therapy with labeled affibody (Id.).

The feasibility of using radiolabeled affibodies for in vivo tumor imaging has been recently demonstrated (Tolmachev et al., 2011, Bioconjugate Chem 22:894-902). A maleimide-derivatized NOTA was conjugated to the anti-HER2 affibody and radiolabeled with ¹¹¹In (Id.). Administration to mice bearing the HER2-expressing DU-145 xenograft, followed by gamma camera imaging, allowed visualization of the xenograft (Id.).

Fynomers can also bind to target antigens with a similar affinity and specificity to antibodies. Fynomers are based on the human Fyn SH3 domain as a scaffold for assembly of binding molecules. The Fyn SH3 domain is a fully human, 63 amino acid protein that can be produced in bacteria with high yields. Fynomers may be linked together to yield a multispecific binding protein with affinities for two or more different antigen targets. Fynomers are commercially available from COVAGEN AG (Zurich, Switzerland).

The skilled artisan will realize that affibodies or fynomers may be used as targeting molecules in the practice of the claimed methods and compositions.

Nanobodies

Nanobodies are single-domain antibodies of about 12-15 kDa in size (about 110 amino acids in length). Nanobodies can selectively bind to target antigens, like full-size antibodies, and have similar affinities for antigens. However, because of their much smaller size, they may be capable of better penetration into solid tumors. The smaller size also contributes to the stability of the nanobody, which is more resistant to pH and temperature extremes than full size antibodies (Van Der Linden et al., 1999, Biochim Biophys Act 1431:37-46). Single-domain antibodies were originally developed following the discovery that camelids (camels, alpacas, llamas) possess fully functional antibodies without light chains (e.g., Hamsen et al., 2007, Appl Microbiol Biotechnol 77:13-22). The heavy-chain antibodies consist of a single variable domain (V_{HH}) and two constant domains (C_{H}2 and C_{H}3). Like antibodies, nanobodies may be developed and used as multivalent and/or bispecific constructs. Humanized forms of nanobodies are in commercial development that are targeted to a variety of target antigens, such as IL-6R, vWF, TNF, RSV, RANKL, IL-17A & F and IgE (e.g., ABLYNX®, Ghent, Belgium), with potential clinical use in cancer, inflammation, infectious disease, Alzheimer's disease, acute coronary syndrome and other disorders (e.g., Saerens et al., 2008, Curr Opin Pharmacol 8:600-8; Muyldermans, 2013, Ann Rev Biochem 82:775-97; Ibanez et al., 2011, J Infect Dis 203:1063-72).

The plasma half-life of nanobodies is shorter than that of full-size antibodies, with elimination primarily by the renal route. Because they lack an Fc region, they do not exhibit complement dependent cytotoxicity.

Nanobodies may be produced by immunization of camels, llamas, alpacas or sharks with target antigen, following by isolation of mRNA, cloning into libraries and screening for antigen binding. Nanobody sequences may be humanized by standard techniques (e.g., Jones et al., 1986, Nature 321: 522, Riechmann et al., 1988, Nature 332: 323, Verhoeyen et al., 1988, Science 239: 1534, Carter et al., 1992, Proc. Nat'l Acad. Sci. USA 89: 4285, Sandhu, 1992, Crit. Rev. Biotech. 12: 437, Singer et al., 1993, J. Immun. 150: 2844). Humanization is relatively straight-forward because of the high homology between camelid and human FR sequences.

In various embodiments, the subject CL2A-SN-38 conjugates may comprise nanobodies for targeted delivery of conjugated drug to cells, tissues, organs or pathogens. Nanobodies of use are disclosed, for example, in U.S. Pat. Nos. 7,807,162; 7,939,277; 8,188,223; 8,217,140; 8,372,398; 8,557,965; 8,623,361 and 8,629,244, the Examples section of each incorporated herein by reference.)

Pre-Targeting

Bispecific or multispecific antibodies may be utilized in pre-targeting techniques. Pre-targeting is a multistep process originally developed to resolve the slow blood clearance of directly targeting antibodies, which contributes to undesirable toxicity to normal tissues such as bone marrow. With pre-targeting, a radionuclide or other therapeutic agent is attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

Targetable Constructs

In certain embodiments, targetable construct peptides labeled with one or more therapeutic or diagnostic agents for use in pre-targeting may be selected to bind to a bispecific antibody with one or more binding sites for a targetable construct peptide and one or more binding sites for a target antigen associated with a disease or condition. Bispecific antibodies may be used in a pretargeting technique wherein the antibody may be administered first to a subject. Sufficient time may be allowed for the bispecific antibody to bind to a target antigen and for unbound antibody to clear from circulation. Then a targetable construct, such as a labeled peptide, may be administered to the subject and allowed to bind to the bispecific antibody and localize at the diseased cell or tissue.

Such targetable constructs can be of diverse structure and are selected not only for the availability of an antibody or fragment that binds with high affinity to the targetable construct, but also for rapid in vivo clearance when used within the pre-targeting method and bispecific antibodies (bsAb) or multispecific antibodies. Hydrophobic agents are best at eliciting strong immune responses, whereas hydrophilic agents are preferred for rapid in vivo clearance. Thus, a balance between hydrophobic and hydrophilic character is established. This may be accomplished, in part, by using hydrophilic chelating agents to offset the inherent hydrophobicity of many organic moieties. Also, sub-units of the targetable construct may be chosen which have opposite solution properties, for example, peptides, which contain amino acids, some of which are hydrophobic and some of which are hydrophilic.

Peptides having as few as two amino acid residues, preferably two to ten residues, may be used and may also be coupled to other moieties, such as chelating agents. The linker should be a low molecular weight conjugate, preferably having a molecular weight of less than 50,000 daltons, and advantageously less than about 20,000 daltons, 10,000 daltons or 5,000 daltons. More usually, the targetable construct peptide will have four or more residues, such as the peptide DOTA-Phe-Lys(HSG)-Tyr-Lys(HSG)-NH₂ (SEQ ID NO: 90), wherein DOTA is 1,4,7,10-tetraazacyclododecane-1,4,7,10-tetraacetic acid and HSG is the histamine succinyl glycyl group. Alternatively, DOTA may be replaced by NOTA (1,4,7-triaza-cyclononane-1,4,7-triacetic acid), TETA (p-bromoacetamido-benzyl-tetraethylaminetetraacetic acid), NETA ([2-(4,7-biscarboxymethyl[1,4,7]triazacyclononan-1-yl-ethyl]-2-carbonylmethyl-amino]acetic acid) or other known chelating moieties. Chelating moieties may be used, for example, to bind to a therapeutic and or diagnostic radionuclide, paramagnetic ion or contrast agent.

The targetable construct may also comprise unnatural amino acids, e.g., D-amino acids, in the backbone structure to increase the stability of the peptide in vivo. In alternative embodiments, other backbone structures such as those constructed from non-natural amino acids or peptoids may be used.

The peptides used as targetable constructs are conveniently synthesized on an automated peptide synthesizer using a solid-phase support and standard techniques of repetitive orthogonal deprotection and coupling. Free amino groups in the peptide, that are to be used later for conjugation of chelating moieties or other agents, are advantageously blocked with standard protecting groups such as a Boc group, while N-terminal residues may be acetylated to increase serum stability. Such protecting groups are well known to the skilled artisan. See Greene and Wuts Protective Groups in Organic Synthesis, 1999 (John Wiley and Sons, N.Y.). When the peptides are prepared for later use within the bispecific antibody system, they are advantageously cleaved from the resins to generate the corresponding C-terminal amides, in order to inhibit in vivo carboxypeptidase activity. Exemplary methods of peptide synthesis are disclosed in the Examples below.

Where pretargeting with bispecific antibodies is used, the antibody will contain a first binding site for an antigen produced by or associated with a target tissue and a second binding site for a hapten on the targetable construct. Exemplary haptens include, but are not limited to, HSG and In-DTPA. Antibodies raised to the HSG hapten are known (e.g. 679 antibody) and can be easily incorporated into the appropriate bispecific antibody (see, e.g., U.S. Pat. Nos. 6,962,702; 7,138,103 and 7,300,644, incorporated herein by reference with respect to the Examples sections). However, other haptens and antibodies that bind to them are known in the art and may be used, such as In-DTPA and the 734 antibody (e.g., U.S. Pat. No. 7,534,431, the Examples section incorporated herein by reference).

Preparation of Immunoconjugates

In preferred embodiments, a therapeutic or diagnostic agent may be covalently attached to an antibody or antibody fragment to form an immunoconjugate. Where the immunoconjugate is to be administered in concentrated form by subcutaneous, intramuscular or transdermal delivery, the skilled artisan will realize that only non-cytotoxic agents may be conjugated to the antibody. Where a second antibody or fragment thereof is administered by a different route, such as intravenously, either before, simultaneously with or after the subcutaneous, intramuscular or transdermal delivery, then the type of diagnostic or therapeutic agent that may be conjugated to the second antibody or fragment thereof is not so limited, and may comprise any diagnostic or therapeutic agent known in the art, including cytotoxic agents.

In some embodiments, a diagnostic and/or therapeutic agent may be attached to an antibody or fragment thereof via a carrier moiety. Carrier moieties may be attached, for example to reduced SH groups and/or to carbohydrate side chains. A carrier moiety can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the carrier moiety can be conjugated via a carbohydrate moiety in the Fc region of the antibody.

Methods for conjugating functional groups to antibodies via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, the Examples section of which is incorporated herein by reference. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); U.S. Pat. Nos. 5,443,953 and 6,254,868, the Examples section of which is incorporated herein by reference. The engineered carbohydrate moiety is used to attach the therapeutic or diagnostic agent.

An alternative method for attaching carrier moieties to a targeting molecule involves use of click chemistry reactions. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. Although the copper catalyst is toxic to living cells, the copper-based click chemistry reaction may be used in vitro for immunoconjugate formation.

A copper-free click reaction has been proposed for covalent modification of biomolecules. (See, e.g., Agard et al., 2004, J Am Chem Soc 126:15046-47.) The copper-free reaction uses ring strain in place of the copper catalyst to promote a [3+2] azide-alkyne cycloaddition reaction (Id.). For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions (Id.).

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.). Examples of such substituted cyclooctynes include difluorinated cyclooctynes, 4-dibenzocyclooctynol and azacyclooctyne (Id.). An alternative copper-free reaction involved strain-promoted alkyne-nitrone cycloaddition to give N-alkylated isoxazolines (Id.). The reaction was reported to have exceptionally fast reaction kinetics and was used in a one-pot three-step protocol for site-specific modification of peptides and proteins (Id.). Nitrones were prepared by the condensation of appropriate aldehydes with N-methylhydroxylamine and the cycloaddition reaction took place in a mixture of acetonitrile and water (Id.). These and other known click chemistry reactions may be used to attach carrier moieties to antibodies in vitro.

Agard et al. (2004, J Am Chem Soc 126:15046-47) demonstrated that a recombinant glycoprotein expressed in CHO cells in the presence of peracetylated N-azidoacetylmannosamine resulted in the bioincorporation of the corresponding N-azidoacetyl sialic acid in the carbohydrates of the glycoprotein. The azido-derivatized glycoprotein reacted specifically with a biotinylated cyclooctyne to form a biotinylated glycoprotein, while control glycoprotein without the azido moiety remained unlabeled (Id.). Laughlin et al. (2008, Science 320:664-667) used a similar technique to metabolically label cell-surface glycans in zebrafish embryos incubated with peracetylated N-azidoacetylgalactosamine. The azido-derivatized glycans reacted with difluorinated cyclooctyne (DIFO) reagents to allow visualization of glycans in vivo.

The Diels-Alder reaction has also been used for in vivo labeling of molecules. Rossin et al. (2010, Angew Chem Int Ed 49:3375-78) reported a 52% yield in vivo between a tumor-localized anti-TAG72 (CC49) antibody carrying a trans-cyclooctene (TCO) reactive moiety and an ¹¹¹In-labeled tetrazine DOTA derivative. The TCO-labeled CC49 antibody was administered to mice bearing colon cancer xenografts, followed 1 day later by injection of ¹¹¹In-labeled tetrazine probe (Id.). The reaction of radiolabeled probe with tumor localized antibody resulted in pronounced radioactivity localization in the tumor, as demonstrated by SPECT imaging of live mice three hours after injection of radiolabeled probe, with a tumor-to-muscle ratio of 13:1 (Id.). The results confirmed the in vivo chemical reaction of the TCO and tetrazine-labeled molecules.

Antibody labeling techniques using biological incorporation of labeling moieties are further disclosed in U.S. Pat. No. 6,953,675 (the Examples section of which is incorporated herein by reference). Such “landscaped” antibodies were prepared to have reactive ketone groups on glycosylated sites. The method involved expressing cells transfected with an expression vector encoding an antibody with one or more N-glycosylation sites in the CH1 or Vκ domain in culture medium comprising a ketone derivative of a saccharide or saccharide precursor. Ketone-derivatized saccharides or precursors included N-levulinoyl mannosamine and N-levulinoyl fucose. The landscaped antibodies were subsequently reacted with agents comprising a ketone-reactive moiety, such as hydrazide, hydrazine, hydroxylamino or thiosemicarbazide groups, to form a labeled targeting molecule. Exemplary agents attached to the landscaped antibodies included chelating agents like DTPA, large drug molecules such as doxorubicin-dextran, and acyl-hydrazide containing peptides. The landscaping technique is not limited to producing antibodies comprising ketone moieties, but may be used instead to introduce a click chemistry reactive group, such as a nitrone, an azide or a cyclooctyne, onto an antibody or other biological molecule.

Modifications of click chemistry reactions are suitable for use in vitro or in vivo. Reactive targeting molecule may be formed either by either chemical conjugation or by biological incorporation. The targeting molecule, such as an antibody or antibody fragment, may be activated with an azido moiety, a substituted cyclooctyne or alkyne group, or a nitrone moiety. Where the targeting molecule comprises an azido or nitrone group, the corresponding targetable construct will comprise a substituted cyclooctyne or alkyne group, and vice versa. Such activated molecules may be made by metabolic incorporation in living cells, as discussed above.

Alternatively, methods of chemical conjugation of such moieties to biomolecules are well known in the art, and any such known method may be utilized. General methods of immunoconjugate formation are disclosed, for example, in U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.

Therapeutic and Diagnostic Agents

In certain embodiments, the antibodies or fragments thereof may be used in combination with one or more therapeutic and/or diagnostic agents. Where the agent is attached to an antibody or fragment thereof to be administered by subcutaneous, intramuscular or transdermal administration of a concentrated antibody formulation, then only non-cytotoxic agents are contemplated. Non-cytotoxic agents may include, without limitation, immunomodulators, cytokines (and their inhibitors), chemokines (and their inhibitors), tyrosine kinase inhibitors, growth factors, hormones and certain enzymes (i.e., those that do not induce local necrosis), or their inhibitors. Where the agent is co-administered either before, simultaneously with or after the subcutaneous, intramuscular or transdermal antibody formulation, then cytotoxic agents may be utilized. An agent may be administered as an immunoconjugate with a second antibody or fragment thereof, or may be administered as a free agent. The following discussion applies to both cytotoxic and non-cytotoxic agents.

Therapeutic agents may be selected from the group consisting of a radionuclide, an immunomodulator, an anti-angiogenic agent, a cytokine, a chemokine, a growth factor, a hormone, a drug, a prodrug, an enzyme, an oligonucleotide, a pro-apoptotic agent, an interference RNA, a photoactive therapeutic agent, a tyrosine kinase inhibitor, a Bruton kinase inhibitor, a sphingosine inhibitor, a cytotoxic agent, which may be a chemotherapeutic agent or a toxin, and a combination thereof. The drugs of use may possess a pharmaceutical property selected from the group consisting of antimitotic, antikinase, alkylating, antimetabolite, antibiotic, alkaloid, anti-angiogenic, pro-apoptotic agents, and combinations thereof.

Exemplary drugs may include, but are not limited to, 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celebrex, chlorambucil, cisplatin (CDDP), Cox-2 inhibitors, irinotecan (CPT-11), SN-38, carboplatin, cladribine, camptothecans, cyclophosphamide, cytarabine, dacarbazine, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR (FUdR-dO), fludarabine, flutamide, farnesyl-protein transferase inhibitors, gemcitabine, hydroxyurea, idarubicin, ifosfamide, L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin, PSI-341, raloxifene, semustine, streptozocin, tamoxifen, taxol, temazolomide (an aqueous form of DTIC), transplatinum, thalidomide, thioguanine, thiotepa, teniposide, topotecan, uracil mustard, vinorelbine, vinblastine, vincristine and vinca alkaloids.

Toxins may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Immunomodulators may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β, -λ or -γ, and stem cell growth factor, such as that designated “S1 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, -λ and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-23, IL-25, LIF, kit-ligand or FLT-3, angiostatin, thrombospondin, endostatin, tumor necrosis factor and lymphotoxin.

Chemokines of use include RANTES, MCAF, MIP 1-alpha, MIP 1-Beta and IP-10.

Radioactive isotopes include, but are not limited to ¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

A variety of tyrosine kinase inhibitors are known in the art and any such known therapeutic agent may be utilized. Exemplary tyrosine kinase inhibitors include, but are not limited to canertinib, dasatinib, erlotinib, gefitinib, imatinib, lapatinib, leflunomide, nilotinib, pazopanib, semaxinib, sorafenib, sunitinib, sutent and vatalanib. A specific class of tyrosine kinase inhibitor is the Bruton tyrosine kinase inhibitor. Bruton tyrosine kinase (Btk) has a well-defined role in B-cell development. Bruton kinase inhibitors include, but are not limited to, PCI-32765 (ibrutinib), PCI-45292, GDC-0834, LFM-A13 and RN486.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Joni et al. (eds.), PHOTODYNAMIC THERAPY OF TUMORS AND OTHER DISEASES (Libreria Progetto 1985); van den Bergh, Chem. Britain (1986), 22:430. Moreover, monoclonal antibodies have been coupled with photoactivated dyes for achieving phototherapy. See Mew et al., J. Immunol. (1983), 130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Corticosteroid hormones can increase the effectiveness of other chemotherapy agents, and consequently, they are frequently used in combination treatments. Prednisone and dexamethasone are examples of corticosteroid hormones.

In certain embodiments, anti-angiogenic agents, such as angiostatin, baculostatin, canstatin, maspin, anti-placenta growth factor (P1GF) peptides and antibodies, anti-vascular growth factor antibodies (such as anti-VEGF and anti-P1GF), anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, interferon-lambda, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline may be of use.

The therapeutic agent may comprise an oligonucleotide, such as a siRNA. The skilled artisan will realize that any siRNA or interference RNA species may be attached to an antibody or fragment thereof for delivery to a targeted tissue. Many siRNA species against a wide variety of targets are known in the art, and any such known siRNA may be utilized in the claimed methods and compositions.

Known siRNA species of potential use include those specific for IKK-gamma (U.S. Pat. No. 7,022,828); VEGF, Flt-1 and Flk-1/KDR (U.S. Pat. No. 7,148,342); Bcl2 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Minis Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL complexes.

Exemplary siRNA species known in the art are listed in Table 6. Although siRNA is delivered as a double-stranded molecule, for simplicity only the sense strand sequences are shown in Table 6.

TABLE 6
Exemplary siRNA Sequences

| Target | Sequence | SEQ ID NO |
|---|---|---|
| VEGF R2 | AATGCGGCGGTGGTGACAGTA | SEQ ID NO: 91 |
| VEGF R2 | AAGCTCAGCACACAGAAAGAC | SEQ ID NO: 92 |
| CXCR4 | UAAAAUCUUCCUGCCCACCdTdT | SEQ ID NO: 93 |
| CXCR4 | GGAAGCUGUUGGCUGAAAAdTdT | SEQ ID NO: 94 |
| PPARC1 | AAGACCAGCCUCUUUGCCCAG | SEQ ID NO: 95 |
| Dynamin 2 | GGACCAGGCAGAAAACGAG | SEQ ID NO: 96 |
| Catenin | CUAUCAGGAUGACGCGG | SEQ ID NO: 97 |
| ElA binding protein | UGACACAGGCAGGCUUGACUU | SEQ ID NO: 98 |
| Plasminogen | GGTGAAGAAGGGCGTCCAA | SEQ ID NO: 99 |
| activator | | |
| K-ras | GATCCGTTGGAGCTGTTGGCGTAGTT | SEQ ID NO: 100 |
| | CAAGAGACTCGCCAACAGCTCCAACT | |
| | TTTGGAAA | |
| Sortilin 1 | AGGTGGTGTTAACAGCAGAG | SEQ ID NO: 101 |
| Apolipoprotein E | AAGGTGGAGCAAGCGGTGGAG | SEQ ID NO: 102 |
| Apolipoprotein E | AAGGAGTTGAAGGCCGACAAA | SEQ ID NO: 103 |
| Bc1-X | UAUGGAGCUGCAGAGGAUGdTdT | SEQ ID NO: 104 |
| Raf-1 | TTTGAATATCTGTGCTGAGAACACA | SEQ ID NO: 105 |
| | GTTCTCAGCACAGATATTCTTTTT | |
| Heat shock | AATGAGAAAAGCAAAAGGTGCCCTGTCTC | SEQ ID NO: 106 |
| transcription factor 2 | | |
| IGFBP3 | AAUCAUCAUCAAGAAAGGGCA | SEQ ID NO: 107 |
| Thioredoxin | AUGACUGUCAGGAUGUUGCdTdT | SEQ ID NO: 108 |
| CD44 | GAACGAAUCCUGAAGACAUCU | SEQ ID NO: 109 |
| MMP14 | AAGCCTGGCTACAGCAATATGCCTGTCTC | SEQ ID NO: 110 |
| MAPKAPK2 | UGACCAUCACCGAGUUUAUdTdT | SEQ ID NO: 111 |
| FGFR1 | AAGTCGGACGCAACAGAGAAA | SEQ ID NO: 112 |
| ERBB2 | CUACCUUUCUACGGACGUGdTdT | SEQ ID NO: 113 |
| BCL2L1 | CTGCCTAAGGCGGATTTGAAT | SEQ ID NO: 114 |
| ABL1 | TTAUUCCUUCUUCGGGAAGUC | SEQ ID NO: 115 |
| CEACAM1 | AACCTTCTGGAACCCGCCCAC | SEQ ID NO: 116 |
| CD9 | GAGCATCTTCGAGCAAGAA | SEQ ID NO: 117 |
| CD151 | CATGTGGCACCGTTTGCCT | SEQ ID NO: 118 |
| Caspase 8 | AACTACCAGAAAGGTATACCT | SEQ ID NO: 119 |
| BRCA1 | UCACAGUGUCCUUUAUGUAdTdT | SEQ ID NO: 120 |
| p53 | GCAUGAACCGGAGGCCCAUTT | SEQ ID NO: 121 |
| CEACAM6 | CCGGACAGTTCCATGTATA | SEQ ID NO: 126 |

The skilled artisan will realize that Table 6 represents a very small sampling of the total number of siRNA species known in the art, and that any such known siRNA may be utilized in the claimed methods and compositions.

Diagnostic agents are preferably selected from the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹⁸F, ⁵²Fe, ¹¹⁰In, ¹¹¹In, ¹⁷⁷Lu, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters.

Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III).

Ultrasound contrast agents may comprise liposomes, such as gas filled liposomes. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerytherin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Methods of Administration

The subject antibodies and immunoglobulins in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active ingredients (i.e., the labeled molecules) are combined in a mixture with one or more pharmaceutically suitable excipients. Sterile phosphate-buffered saline is one example of a pharmaceutically suitable excipient. Other suitable excipients are well known to those in the art. See, e.g., Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The preferred route for administration of the compositions described herein is parenteral injection, more preferably by subcutaneous, intramuscular or transdermal delivery. Other forms of parenteral administration include intravenous, intraarterial, intralymphatic, intrathecal, intraocular, intracerebral, or intracavitary injection. In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hanks' solution. Nonaqueous excipients such as fixed oils and ethyl oleate may also be used. An alternative excipient is 5% dextrose in saline. The excipient may contain minor amounts of additives such as substances that enhance isotonicity and chemical stability, including buffers and preservatives.

Formulated compositions comprising antibodies can be used for subcutaneous, intramuscular or transdermal administration. Compositions can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The compositions may be administered in solution. The formulation thereof should be in a solution having a suitable pharmaceutically acceptable buffer such as phosphate, TRIS (hydroxymethyl)aminomethane-HCl or citrate and the like. Buffer concentrations should be in the range of 1 to 100 mM. The formulated solution may also contain a salt, such as sodium chloride or potassium chloride in a concentration of 50 to 150 mM. An effective amount of a stabilizing agent such as mannitol, trehalose, sorbitol, glycerol, albumin, a globulin, a detergent, a gelatin, a protamine or a salt of protamine may also be included.

The dosage of an administered antibody for humans will vary depending upon such factors as the patient's age, weight, height, sex, general medical condition and previous medical history. Typically, it is desirable to provide the recipient with a dosage of antibody that is in the range of from about 1 mg to 600 mg as a single infusion or single or multiple injections, although a lower or higher dosage also may be administered. Typically, it is desirable to provide the recipient with a dosage that is in the range of from about 50 mg per square meter (m²) of body surface area or 70 to 85 mg of the antibody for the typical adult, although a lower or higher dosage also may be administered. Examples of dosages of antibodies that may be administered to a human subject are 1 to 1,000 mg, more preferably 1 to 70 mg, most preferably 1 to 20 mg, although higher or lower doses may be used. Dosages may be repeated as needed, for example, once per week for 4-10 weeks, preferably once per week for 8 weeks, and more preferably, once per week for 4 weeks. It may also be given less frequently, such as every other week for several months, or more frequently, such as twice weekly or by continuous infusion.

More recently, subcutaneous administration of veltuzumab has been given to NHL patients in 4 doses of 80, 160 or 320 mg, repeated every two weeks (Negrea et al., 2011, Haematologica 96:567-73). Only occasional, mild to moderate and transient injection reactions were observed, with no other safety issues (Id.). The objective response rate (CR+CRu+PR) was 47%, with a CR/CRu (complete response) rate of 24% (Id.). Interestingly, the 80 mg dosage group showed the highest percentage of objective response (2/3, 67%), with one of three patients showing a complete response (Id.). Four out of eight objective responses continued for 60 weeks (Id.). All serum samples evaluated for HAHA were negative (Id.). Although the low sample population reported in this study precludes any definitive conclusions on optimal dosing, it is apparent that therapeutic response was observed at the lowest dosage tested (80 mg).

In certain alternative embodiments, the antibody may be administered by transdermal delivery. Different methods of transdermal delivery are known in the art, such as by transdermal patches or by microneedle devices, and any such known method may be utilized. In an exemplary embodiment, transdermal delivery may utilize a delivery device such as the 3M hollow Microstructured Transdermal System (hMTS) for antibody based therapeutics. The hMTS device comprises a 1 cm² microneedle array consisting of 18 hollow microneedles that are 950 microns in length, which penetrate approximately 600-700 microns into the dermal layer of the skin where there is a high density of lymphatic channels. A spring-loaded device forces the antibody composition from a fluid reservoir through the microneedles for delivery to the subject. Only transient erythema and edema at the injection site are observed (Burton et al., 2011, Pharm Res 28:31-40). The hMTS device is not perceived as a needle injector, resulting in improved patient compliance.

In alternative embodiments, transdermal delivery of peptides and proteins may be achieved by (1) coadministering with a synthetic peptide comprising the amino acid sequence of ACSSSPSKHCG (SEQ ID NO:123) as reported by Chen et al. (Nat Biotechnol 2006; 24: 455-460) and Carmichael et al. (Pain 2010; 149:316-324); (2) coadministering with arginine-rich intracellular delivery peptides as reported by Wang et al. (BBRC 2006; 346: 758-767); (3) coadminstering with either AT1002 (FCIGRLCG, SEQ ID NO:124) or Tat (GRKKRRNRRRCG, SEQ ID NO:125) as reported by Uchida et al. (Chem Pharm Bull 2011; 59:196); or (4) using an adhesive transdermal patch as reported by Jurynczyk et al (Ann Neurol 2010; 68:593-601). In addition, transdermal delivery of negatively charged drugs may be facilitated by combining with the positively charged, pore-forming magainin peptide as reported by Kim et al. (Int J Pharm 2008; 362:20-28).

In preferred embodiments where the antibody is administered subcutaneously, intramuscularly or transdermally in a concentrated formulation, the volume of administration is preferably limited to 3 ml or less, more preferably 2 ml or less, more preferably 1 ml or less. The use of concentrated antibody formulations allowing low volume subcutaneous, intramuscular or transdermal administration is preferred to the use of more dilute antibody formulations that require specialized devices and ingredients (e.g., hyaluronidase) for subcutaneous administration of larger volumes of fluid, such as 10 ml or more. The subcutaneous, intramuscular or transdermal delivery may be administered as a single administration to one skin site or alternatively may be repeated one or more times, or even given to more than one skin site in one therapeutic dosing session. However, the more concentrated the formulation, the lower the volume injected and the fewer injections will be needed for each therapeutic dosing.

Methods of Use

In preferred embodiments, the antibodies are of use for therapy of cancer. Examples of cancers include, but are not limited to, carcinoma, lymphoma, blastoma, glioma, melanoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, neuroblastoma, cervical cancer, ovarian cancer, liver cancer, bladder cancer, hepatoma, breast cancer, colon cancer, rectal cancer, endometrial cancer or uterine carcinoma, salivary gland carcinoma, kidney or renal cancer, prostate cancer, vulval cancer, thyroid cancer, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: acute childhood lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignancies, anal cancer, astrocytoma, bile duct cancer, bladder cancer, bone cancer, brain stem glioma, brain tumors, breast cancer, cancer of the renal pelvis and ureter, central nervous system (primary) lymphoma, central nervous system lymphoma, cerebellar astrocytoma, cerebral astrocytoma, cervical cancer, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood cerebral astrocytoma, childhood extracranial germ cell tumors, childhood Hodgkin's disease, childhood Hodgkin's lymphoma, childhood hypothalamic and visual pathway glioma, childhood lymphoblastic leukemia, childhood medulloblastoma, childhood non-Hodgkin's lymphoma, childhood pineal and supratentorial primitive neuroectodermal tumors, childhood primary liver cancer, childhood rhabdomyosarcoma, childhood soft tissue sarcoma, childhood visual pathway and hypothalamic glioma, chronic lymphocytic leukemia, chronic myelogenous leukemia, colon cancer, cutaneous T-cell lymphoma, endocrine pancreas islet cell carcinoma, endometrial cancer, ependymoma, epithelial cancer, esophageal cancer, Ewing's sarcoma and related tumors, exocrine pancreatic cancer, extracranial germ cell tumor, extragonadal germ cell tumor, extrahepatic bile duct cancer, eye cancer, female breast cancer, Gaucher's disease, gallbladder cancer, gastric cancer, gastrointestinal carcinoid tumor, gastrointestinal tumors, germ cell tumors, gestational trophoblastic tumor, hairy cell leukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, kidney cancer, laryngeal cancer, lip and oral cavity cancer, liver cancer, lung cancer, lymphoproliferative disorders, macroglobulinemia, male breast cancer, malignant mesothelioma, malignant thymoma, medulloblastoma, melanoma, mesothelioma, metastatic occult primary squamous neck cancer, metastatic primary squamous neck cancer, metastatic squamous neck cancer, multiple myeloma, multiple myeloma/plasma cell neoplasm, myelodysplastic syndrome, myelogenous leukemia, myeloid leukemia, myeloproliferative disorders, nasal cavity and paranasal sinus cancer, nasopharyngeal cancer, neuroblastoma, non-Hodgkin's lymphoma during pregnancy, nonmelanoma skin cancer, non-small cell lung cancer, occult primary metastatic squamous neck cancer, oropharyngeal cancer, osteo-/malignant fibrous sarcoma, osteosarcoma/malignant fibrous histiocytoma, osteosarcoma/malignant fibrous histiocytoma of bone, ovarian epithelial cancer, ovarian germ cell tumor, ovarian low malignant potential tumor, pancreatic cancer, paraproteinemias, purpura, parathyroid cancer, penile cancer, pheochromocytoma, pituitary tumor, plasma cell neoplasm/multiple myeloma, primary central nervous system lymphoma, primary liver cancer, prostate cancer, rectal cancer, renal cell cancer, renal pelvis and ureter cancer, retinoblastoma, rhabdomyosarcoma, salivary gland cancer, sarcoidosis sarcomas, Sezary syndrome, skin cancer, small cell lung cancer, small intestine cancer, soft tissue sarcoma, squamous neck cancer, stomach cancer, supratentorial primitive neuroectodermal and pineal tumors, T-cell lymphoma, testicular cancer, thymoma, thyroid cancer, transitional cell cancer of the renal pelvis and ureter, transitional renal pelvis and ureter cancer, trophoblastic tumors, ureter and renal pelvis cell cancer, urethral cancer, uterine cancer, uterine sarcoma, vaginal cancer, visual pathway and hypothalamic glioma, vulvar cancer, Waldenstrom's macroglobulinemia, Wilms' tumor, and any other hyperproliferative disease, besides neoplasia, located in an organ system listed above.

The methods and compositions described and claimed herein may be used to detect or treat malignant or premalignant conditions. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W.B. Saunders Co., Philadelphia, pp. 68-79 (1976)).

Dysplasia is frequently a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. Dysplastic disorders which can be detected include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be detected and/or treated include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

The exemplary conditions listed above that may be treated are not limiting. The skilled artisan will be aware that antibodies or antibody fragments are known for a wide variety of conditions, such as autoimmune disease, graft-versus-host-disease, organ transplant rejection, cardiovascular disease, neurodegenerative disease, metabolic disease, cancer, infectious disease and hyperproliferative disease.

Exemplary autoimmune diseases include acute idiopathic thrombocytopenic purpura, chronic immune thrombocytopenia, dermatomyositis, Sydenham's chorea, myasthenia gravis, systemic lupus erythematosus, lupus nephritis, rheumatic fever, polyglandular syndromes, bullous pemphigoid, pemphigus vulgaris, juvenile diabetes mellitus, Henoch-Schonlein purpura, post-streptococcal nephritis, erythema nodosum, Takayasu's arteritis, ANCA-associated vasculitides, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjögren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis and fibrosing alveolitis.

Kits

Various embodiments may concern kits containing components suitable for treating diseased tissue in a patient. Exemplary kits may contain at least one anti-B-cell antibody or fragment thereof as described herein. A device capable of delivering the kit components by injection, for example, a syringe for subcutaneous injection, may be included. Where transdermal administration is used, a delivery device such as hollow microneedle delivery device may be included in the kit. Exemplary transdermal delivery devices are known in the art, such as 3M's hollow Microstructured Transdermal System (hMTS), and any such known device may be used.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Alternatively, the concentrated antibody may be delivered and stored as a liquid formulation. Other containers that may be used include, but are not limited to, a pouch, tray, box, tube, or the like. Kit components may be packaged and maintained sterilely within the containers. Another component that can be included is instructions to a person using a kit for its use.

### EXAMPLES

### Example 1

### Epratuzumab-Induced Trogocytosis of BCR-Response Modulating Proteins Ex Vivo

The humanized anti-CD22 antibody, epratuzumab, has demonstrated therapeutic activity in clinical trials of patients with non-Hodgkin lymphoma (NHL), acute lymphoblastic leukemia, primary Sjögren's syndrome, and systemic lupus erythematosus (SLE). Thus, epratuzumab offers a promising option for CD22-targeted immunotherapy of B-cell lymphomas and autoimmune diseases. However, its mechanism of action (MOA) remains incompletely understood to-date. Because epratuzumab has modest, but significant, antibody-dependent cell-mediated cytotoxicity and negligible complement-dependent cytotoxicity when evaluated in vitro, and its moderate depletion of circulating B cells in patients (35% on average) may be overestimated due to use of CD19⁺ cells to measure B cells by flow cytometry (discussed below), the therapeutic action of epratuzumab in vivo may not result from B-cell depletion. We investigated whether ligation of epratuzumab to CD22 could modulate other surface molecules on B cells. In particular, we focused on those surface molecules involved in regulating antigen-specific B-cell receptor (BCR) signaling, since modulation of such molecules may lead to altered B-cell functions that ultimately mitigate symptoms of autoimmune or other diseases. With regard to its function of killing malignant B cells expressing CD22, our studies have shown that these effects are more related to the BCR signaling pathway than effector-cell function.

As discussed below, epratuzumab induced a substantial reduction of CD22, along with CD19, CD21, CD20, and CD79b, on the surface of B cells in peripheral blood mononuclear cells (PBMCs) obtained from normal donors or lupus patients, and three NHL Burkitt cell lines (Daudi, Raji, and Ramos) spiked into normal PBMCs. The intriguing observation that only CD22, but not other surface markers, was appreciably decreased by epratuzumab in isolated NHL cells prompted us to assess the role of FcγR-bearing effector cells, with the finding that epratuzumab effectively mediates trogocytosis [a process whereby cells binding to antigen-presenting cells extract surface molecules from these cells and express them on their own surface] of multiple surface proteins from B cells to monocytes, NK cells, and neutrophils. This mechanism of action may explain the limited effectiveness of high doses of epratuzumab compared to lower doses in patients with SLE.

Peripheral blood mononuclear cells (PBMCs) obtained from healthy donors were incubated overnight (16-24 h) with 10 μg/mL of either epratuzumab or an isotype control mAb (hMN-14) and the relative levels of various antigens on the surface of the B cells were analyzed by flow cytometry. PBMCs from heparinized whole blood of normal donors were isolated by density gradient centrifugation on UNI-SEP tubes (Novamed Ltd, Israel). PBMCs were reconstituted in RPMI media supplemented with 10% heat inactivated fetal bovine serum and plated at a cell density of 1.5×10⁶/mL in non-tissue culture treated 48-well plates. Epratuzumab or hMN-14 were added to triplicate wells at a final concentration of 10 μg/mL and incubated overnight (16-20 h) before staining with fluorescent-labeled primary antibodies (Biolegend) following the manufacturers suggested protocols. Stained cells were analyzed by flow cytometry on a FACSCALIBUR® (BD Biosciences) using Flowjo (V7.6.5) software. Initially, the lymphocyte population was gated by side vs. forward scattering, and B cells were further gated from this population with the CD19 signal. The mean fluorescence intensity (MFI), obtained with fluorochrome-conjugated antibodies to various cell surface antigens, on the gated B cells was calculated following treatment with epratuzumab, hMN-14 or without antibody. PBMCs from 16 healthy donors were assessed in various experiments.

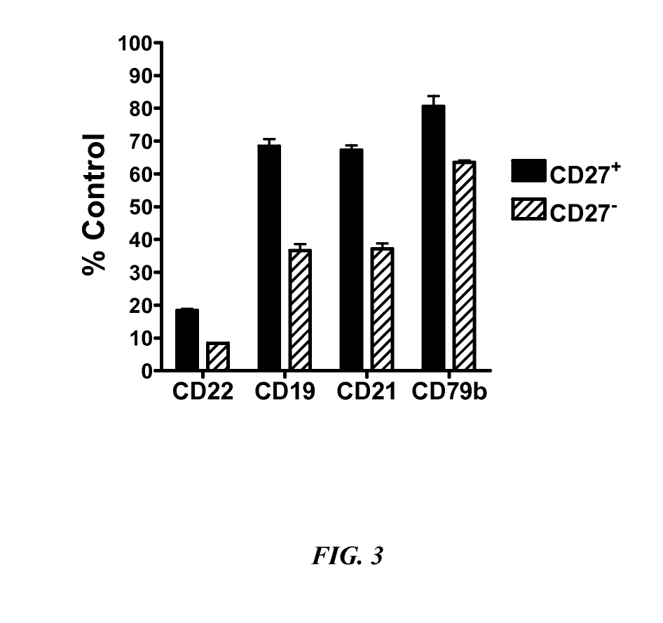

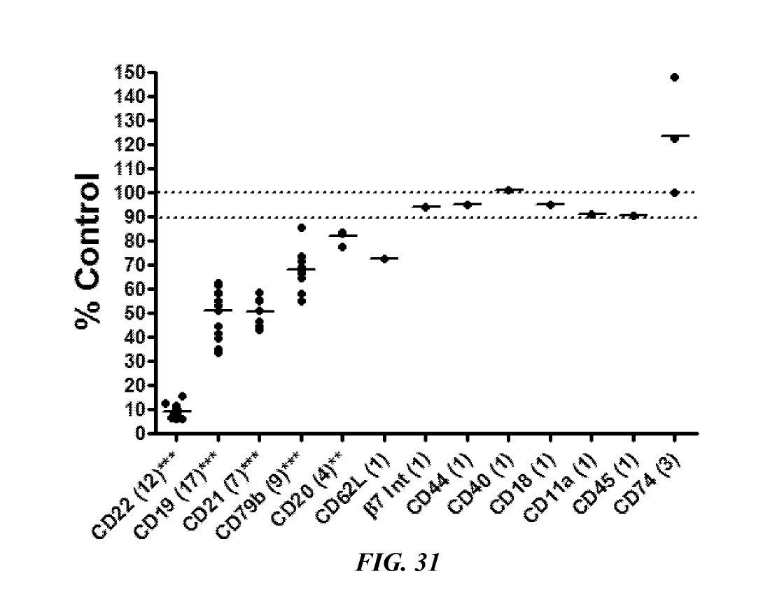

Treatment with the control mAb (hMN-14) did not affect the levels of any of the tested proteins and resulted in MFI measurements that were very similar to untreated samples. Alternatively, epratuzumab significantly reduced the levels of key BCR-regulating proteins, including CD22, CD19, CD21 and CD79b, which were reduced to 10, 50, 52 and 70%, respectively, of the level of untreated or control mAb (FIG. 1). CD20 (82%) and CD62L (73%) also were reduced, but to a lesser extent. Other surface proteins including CD27 (on CD27⁺ B cells), CD40, CD44, CD45, β7 integrin and LFA-1 (CD11a and CD18) were affected minimally (<10% change) by epratuzumab. Similar data with slightly higher sample numbers is shown in FIG. 31. CD27⁻ naive B cells were more responsive to epratuzumab compared to CD27⁺ memory B cells, as shown with PBMCs as shown for CD19 from 3 different healthy donors (FIG. 2). CD22, CD21 and CD79b were also reduced to a greater extent on CD27⁻ cells (FIG. 3). The effect was essentially complete within a few hours. The reductions in surface CD19 and CD21 were not significantly different following 2-h or overnight treatment (FIG. 4).

### Example 2

### Effect of Various B Cell-Targeting Antibodies

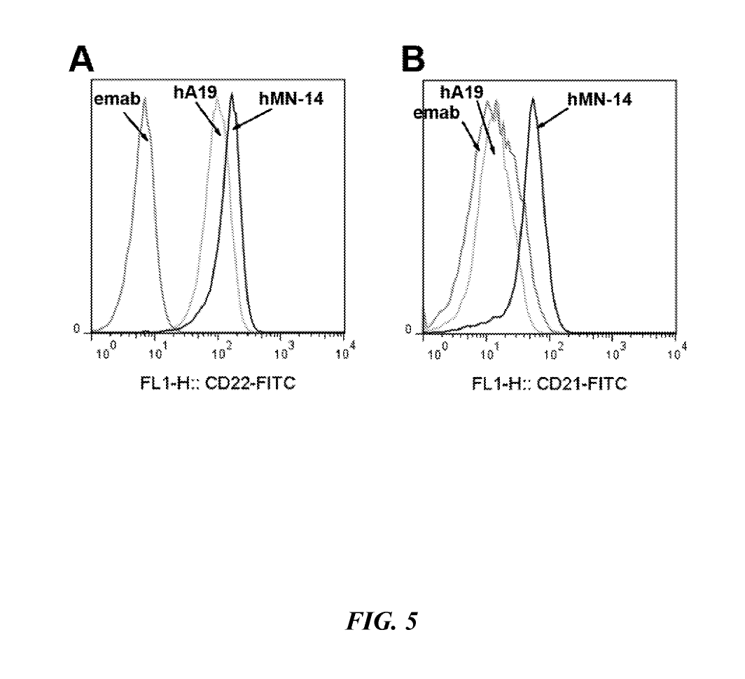

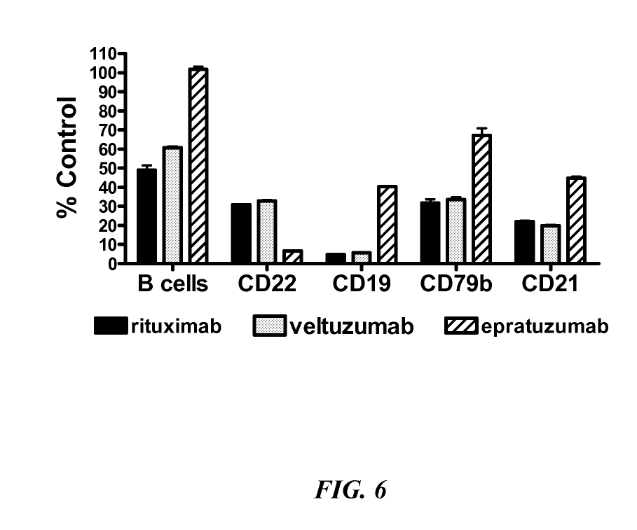

Compared to epratuzumab, a humanized antibody to CD19 (hA19) moderately reduced the level of CD22 on B cells (66% of control) within PBMCs (FIG. 5). Although treatment with hA19 precluded measurement of CD19, that hA19 lowered the level of CD21, to a similar level as epratuzumab, suggests that a concomitant reduction in CD19 also is likely. The CD20-targeting mAbs rituximab and veltuzumab each diminished CD19, CD21 and CD79b to a greater extent than epratuzumab (FIG. 6). Rituximab also reduced CD22, but to a lesser extent than epratuzumab. Notably, rituximab and veltuzumab (at 10 μg/mL) reduced the B cell count by 50%, and 40%, where epratuzumab did not cause significant B cell depletion, either at 10 μg/mL or 1 mg/mL. Unlike rituximab, which reduces the same antigens via trogocytosis, but also potently kills B cells, epratuzumab does not deplete B cells ex vivo.

### Example 3

### Dose-Dependent Trogocytosis with Epratuzumab

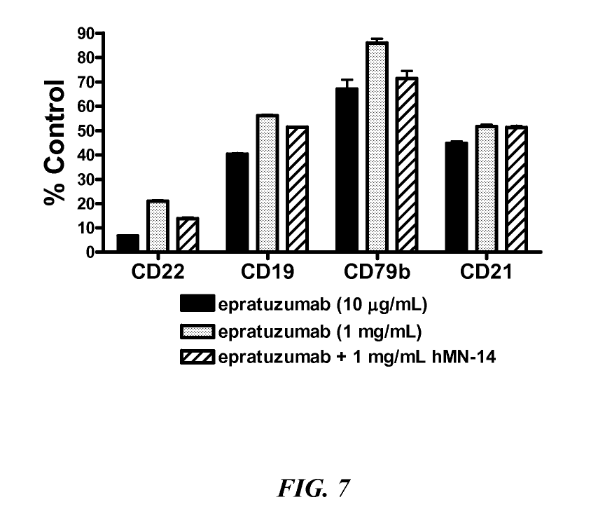

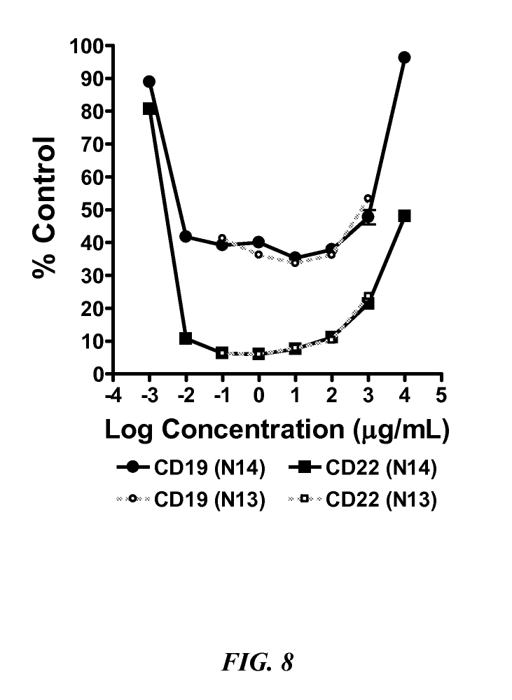

The effect of epratuzumab on the cell surface levels of CD19, CD21, CD22 and CD79b was compared using the standard (10 μg/mL) concentration with a 100-fold higher concentration (1 mg/mL). An additional treatment included 10 μg/mL epratuzumab combined with 1 mg/mL hMN-14. Compared to the lower concentration of epratuzumab (10 μg/mL), the higher concentration (1 mg/mL) resulted in significantly (P<0.02) less reduction in CD22, CD19, CD21 and CD79b (FIG. 7). Competition with high concentration (1 mg/mL) hMN-14 significantly (P<0.003) reduced the effect of epratuzumab (10 mg/mL) on CD22 and CD19, but to a lesser extent than high-dose epratuzumab. A titration experiment, where normal PBMCs were incubated overnight with epratuzumab at concentrations ranging from 0.1-1000 μg/mL, confirmed that doses approaching 1 mg/mL dampened the effect (FIG. 8, donor N13, dashed curves). A second titration covering 8 logs (1 ng/mL-10 mg/mL) produced a classic U-shaped curve with substantial dampening at concentrations lower than 10 ng/mL or greater than 1 mg/mL (FIG. 8, donor N14, solid curves). The reduction of both CD22 and CD19 on B cells within PBMCs was similar over a wide concentration range (10 ng/mL-100 μg/mL) of epratuzumab.

### Example 4

### The Fc is Required for Trogocytosis

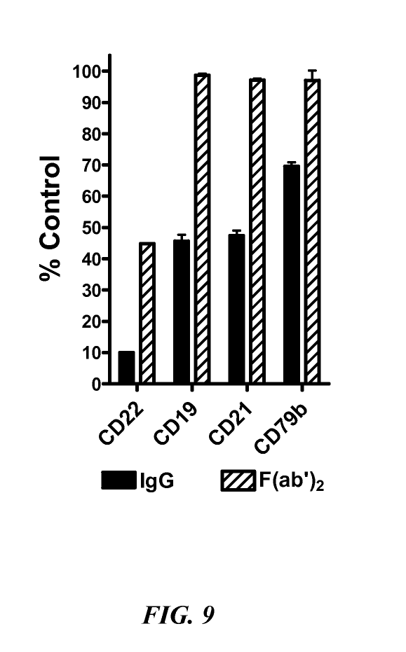

An F(ab′)₂ fragment of epratuzumab, which was prepared by pepsin digestion, reduced CD22 moderately (45% control), compared to the full IgG (10% control), and had no effect on CD19, CD21 and CD79b (FIG. 9). The loss of CD22 can be attributed to internalization of the antibody/antigen complex, which is a well established phenomenon associated with epratuzumab, and not due to trogocytosis. That CD19, CD21 and CD79 are not affected by the F(ab′)₂ indicates that no trogocytosis is induced by the Fc-lacking antibody fragment. A similar finding was observed when PBMCs from lupus patients were used instead of from healthy donors (Example 10).

### Example 5

### Effector Cells are Required for Epratuzumab-Induced Trogocytosis

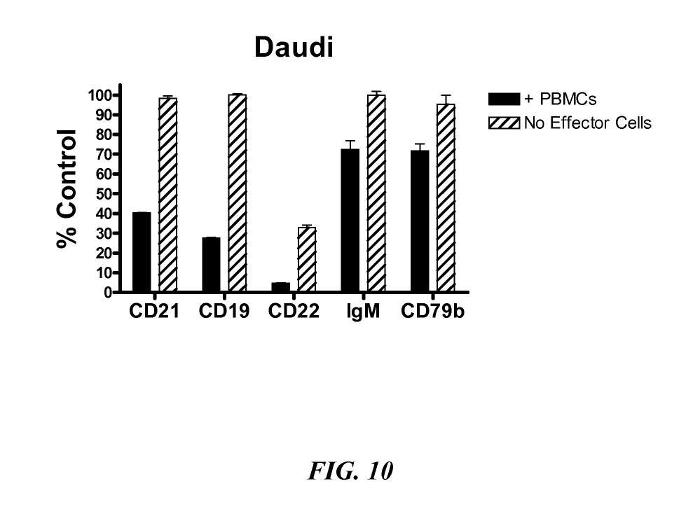

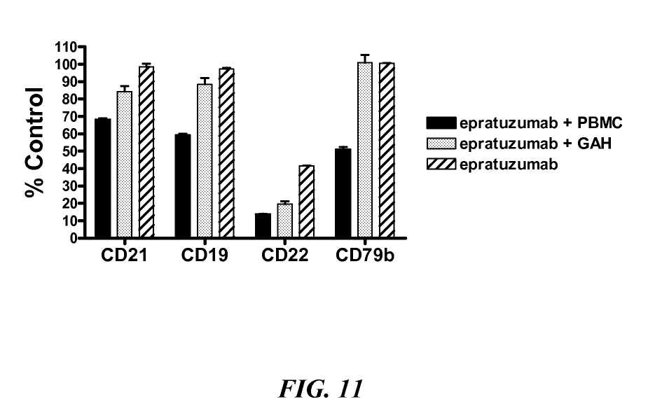

B cell lymphoma cell lines were used as “isolated B cells” that were evaluated for epratuzumab induced trogocytosis. In vitro, epratuzumab induced an intermediate reduction (33% control) of CD22 on the surface of isolated Daudi Burkitt lymphoma cells, and did not affect the levels of other markers (FIG. 10). In an ex vivo setting, where Daudi were spiked into PBMCs from a healthy donor, epratuzumab minimized CD22 (<5% control) and significantly (P<0.0001) reduced CD19 (28% control), CD21 (40% control), CD79b (72% control) and surface IgM (73% control). Similar results were obtained with Raji lymphoma cells, where CD19, CD21 and CD79b were diminished by epratuzumab only in the presence of PBMCs. (FIG. 11). The addition of a crosslinking second antibody resulted in only a modest reduction of CD19, CD21 and CD79b. That the effect only was observed in the presence of PBMCs, and it was not accomplished in the presence of PBMCs with a F(ab′)₂ fragment (Example 4) or with a crosslinking second antibody in place of PBMCs, indicates that effector cells bearing Fc receptors are involved in the epratuzumab-induced trogocytosis process.

### Example 6

### Monocytes, but not T Cells can Modulate Epratuzumab-Induced Trogocytosis

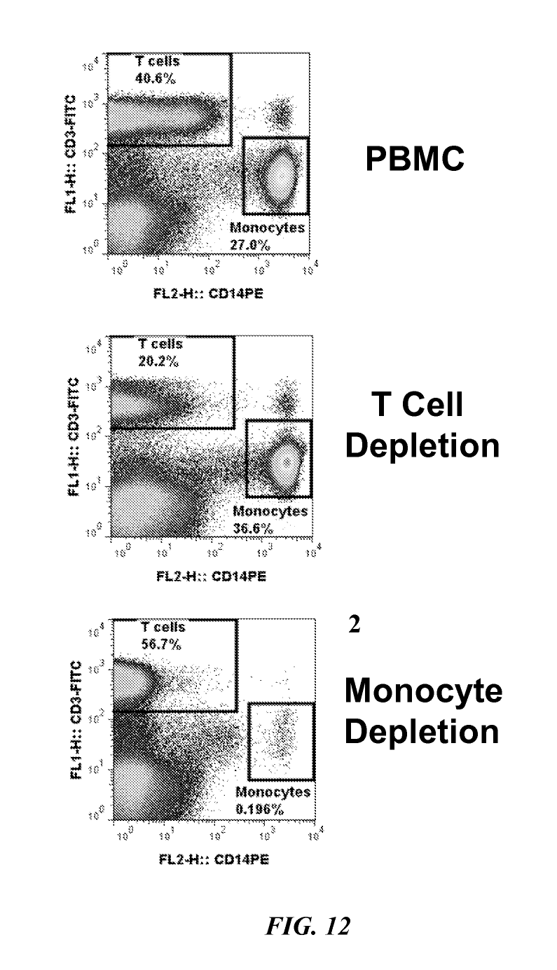

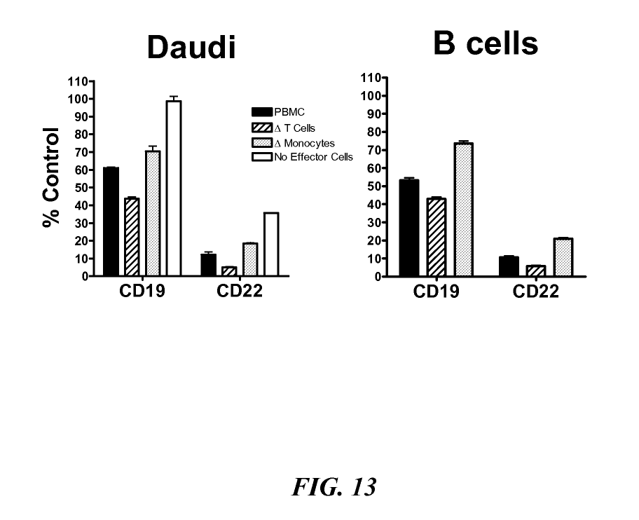

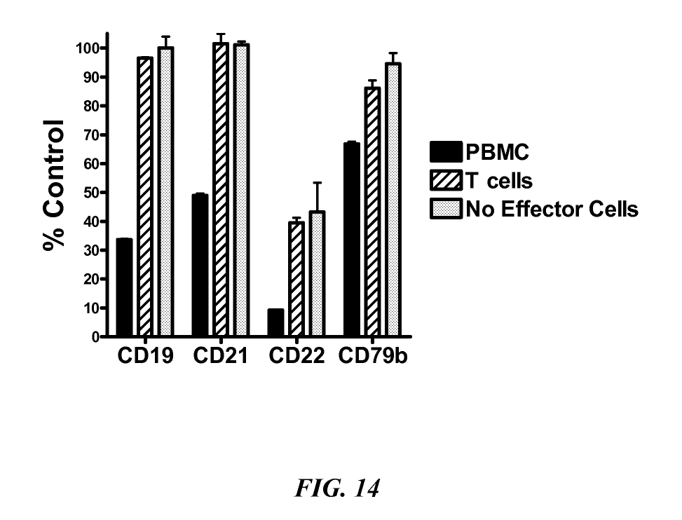

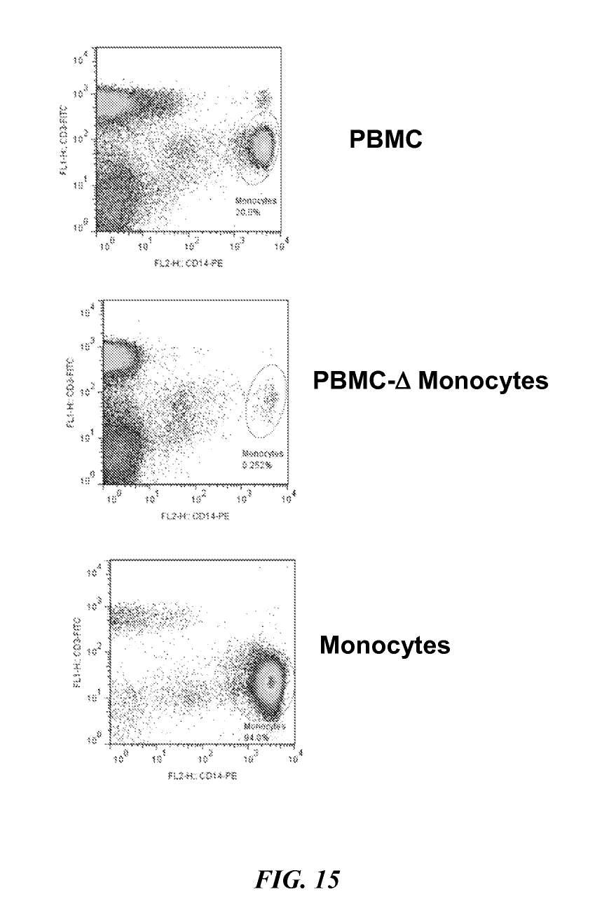

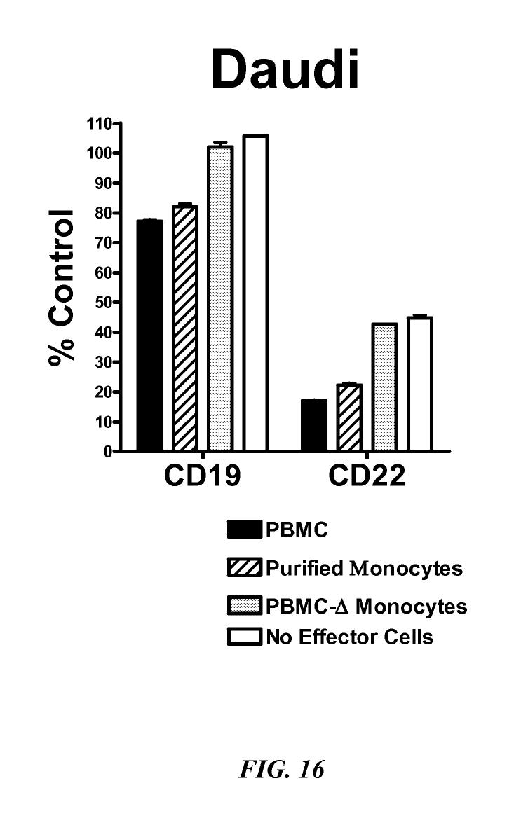

Combined, T cells and monocytes comprise approximately 70-80% of the total PBMCs. The ability of PBMC fractions, which were depleted of either T cells or monocytes using MACS separation technology (Miltenyi Biotec) with magnetically labeled microbeads in an LS or MS column, were evaluated for epratuzumab-induced reduction of CD22 and CD19 on Daudi and normal B cells. For this experiment the ratio of total effector cells to Daudi was held constant. Therefore, removal of a specific cell type resulted in increased numbers of the remaining cell types (FIG. 12). Depletion of T cells was only 50% efficient; however, this resulted in a 10% increase in monocytes and other cell types. The T-cell-depleted PBMCs were significantly more active than total PBMCs, indicating that T cells are not involved (FIG. 13). Indeed, purified T cells were not capable of affecting the epratuzumab-induced reduction of CD19 or CD21 on Daudi (FIG. 14). Conversely, depletion of monocytes, which was 99% efficient (FIG. 12), significantly dampened the reduction of both CD19 and CD22 on either Daudi or B cells (FIG. 13), implicating the involvement of monocytes. That there was appreciable reduction of CD19 with the monocyte-depleted PBMCs, suggests the participation of additional cell types. In a subsequent experiment, purified monocytes (94%, FIG. 15) induced a similar decrease in CD19 as the whole PBMCs, whereas the remaining monocyte-depleted PBMCs had minimal effect, comparable to the levels measured without effector cells (FIG. 16). A similar pattern was observed for CD22. This particular donor gave relatively weak activity (25% reduction in CD19) compared to most others, where we have typically observed a 40-60% reduction in CD19. Nonetheless, the results support the key role of monocytes among PBMCs.

### Example 7

### Epratuzumab-Induced Trogocytosis with Monocytes

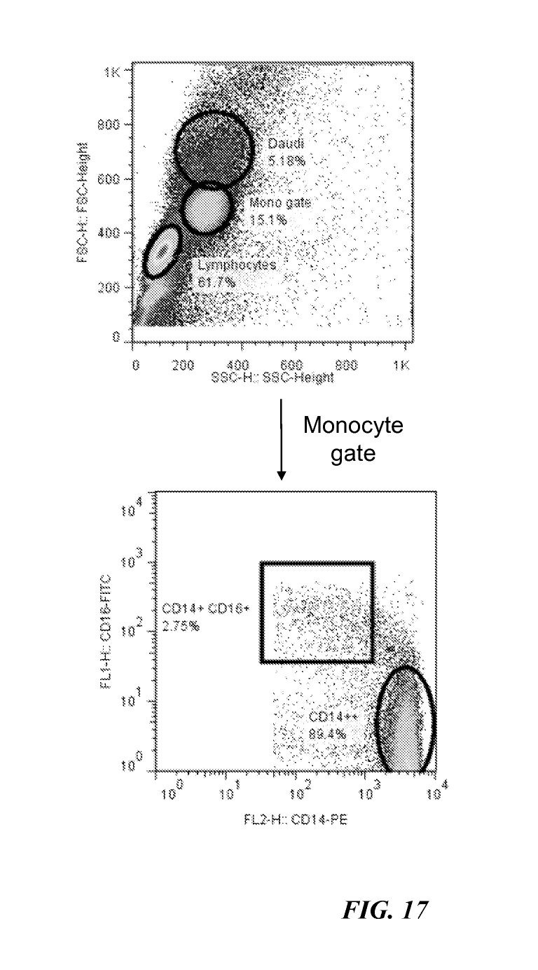

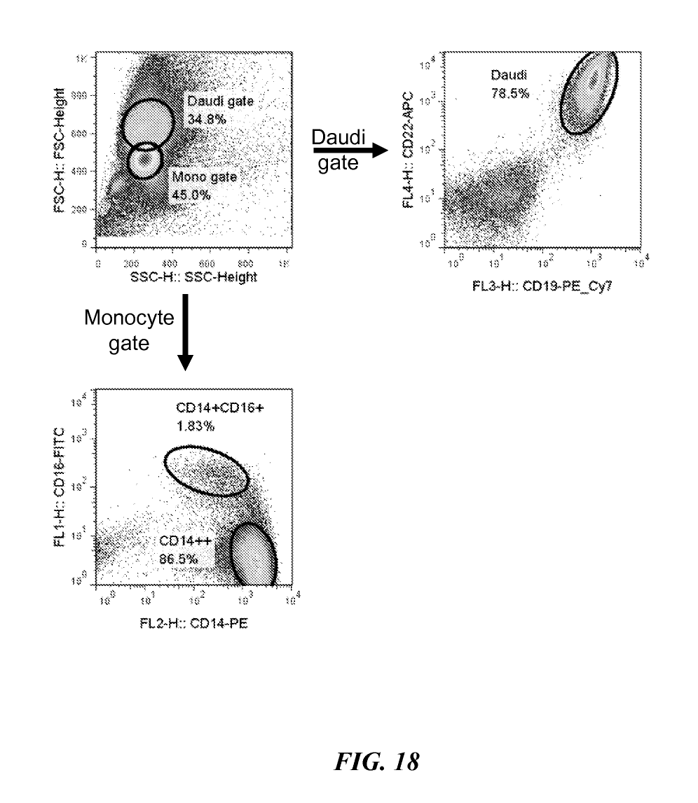

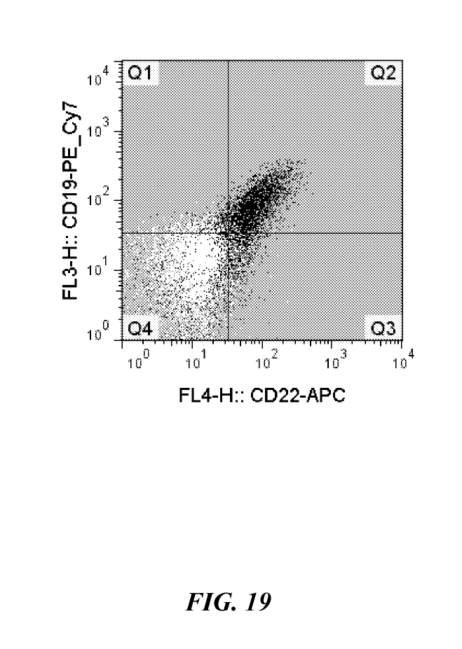

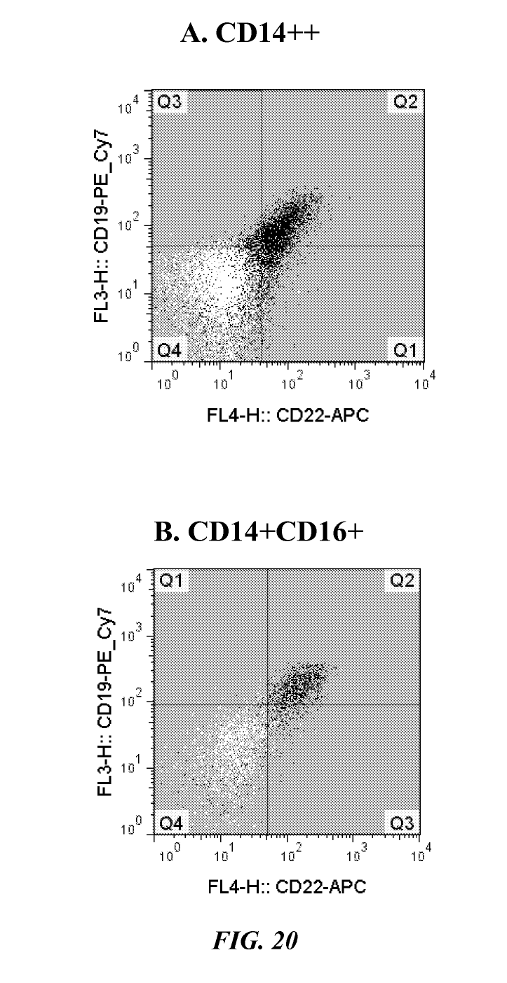

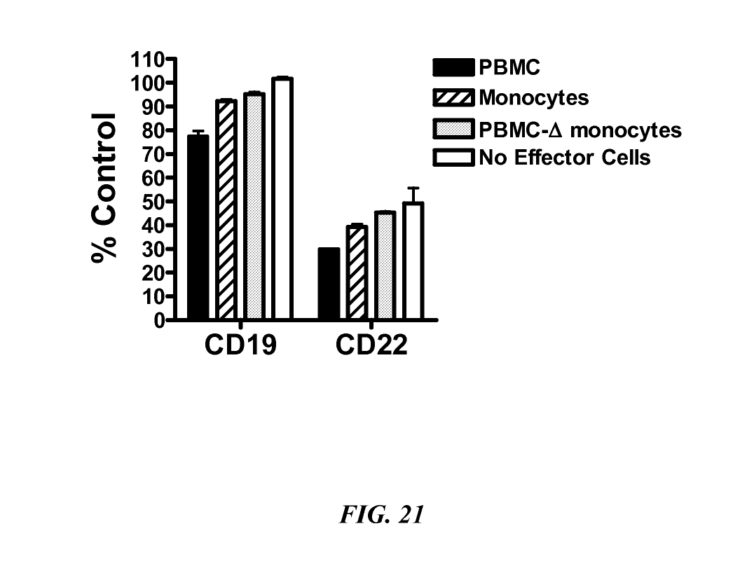

Trogocytosis involves the transfer of membrane components from one cell to another. To determine if the loss of surface antigen on B cells is due to their transfer to effector cells (trogocytosis), Daudi cells were mixed with PBMCs (FIG. 17), purified monocytes (FIG. 18) or monocyte-depleted PBMCs, and treated with epratuzumab or the isotype control for 1 h. Daudi, monocyte and lymphocyte populations were gated by forword vs. side scattering. When mixed with Daudi cells and treated with epratuzumab, but not the isotype control mAb, purified monocytes (CD14 positive cells) stained positive for either CD22 (56.6% positive) and CD19 (52.4% positive), with 44% positive for both (FIG. 19). Treatment with an isotype control mAb resulted in only 1.6% double positive monocytes. The monocytes were further gated into CD14⁺⁺ (˜90%) and CD14⁺CD16⁺ (˜10%) sub-populations (FIG. 17 and FIG. 18). The CD14⁺CD16⁺ monocytes (FIG. 20A) exhibited more activity (66.4% CD19⁺CD22⁺) compared to the more abundant CD14⁺⁺ (31.4%) cells (FIG. 20B). Even after only 1 h, CD19 and CD22 were specifically reduced from Daudi cells when treated with epratuzumab in the presence of PBMCs or purified monocytes (FIG. 21).

Trogocytosis by monocytes induced by epratuzumab was confirmed by fluorescence microscopy. Purified monocytes (membrane-labeled with a red fluorochrome), were mixed 1:1 with Daudi cells (membrane-labeled with a green fluorochrome) and treated with labetuzumab or epratuzumab. Images were captured over 30 min using a 40× objective lens with 115× camera zoom. Incubation of the cell mixture with labetuzumab had no affect, since cells were observed predominantly (>99%) as single cells after 30 min (not shown). Even when cells were juxtaposed, there was no evidence of immunological synapse formation or trogocytosis (not shown). Addition of epratuzumab to the cell mixture resulted in immunological synapse formation between Daudi and monocytes within 10 min (not shown), and subsequent trogocytosis of green-stained Daudi membrane components to the redstained monocytes (not shown). After 30 min, more than 50% of each cell type was associated in various configurations including 1:1, one monocyte with multiple Daudi cells, multiple monocytes with one Daudi cell, and mixed cell clusters (not shown). We conclude that epratuzumab induces formation of immunological synapses between B cells and effector cells by binding to CD22 on B cells and to Fcγ receptors on effector cells (monocytes, NK cells, granulocytes).

### Example 8

### Epratuzumab-Induced Trogocytosis with NK Cells

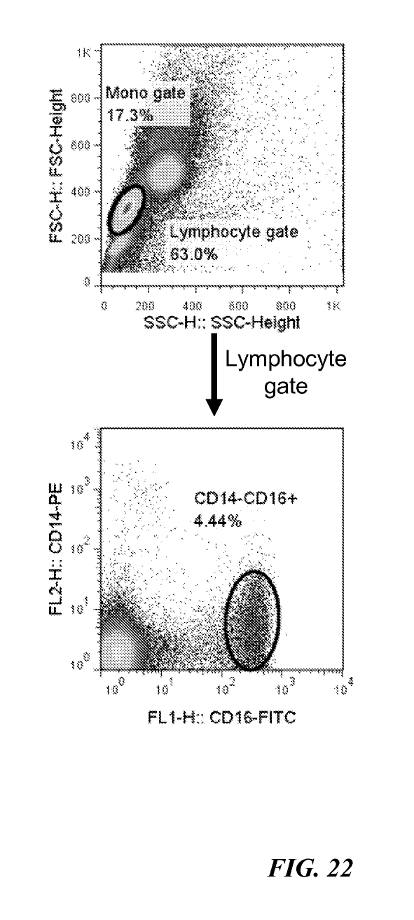

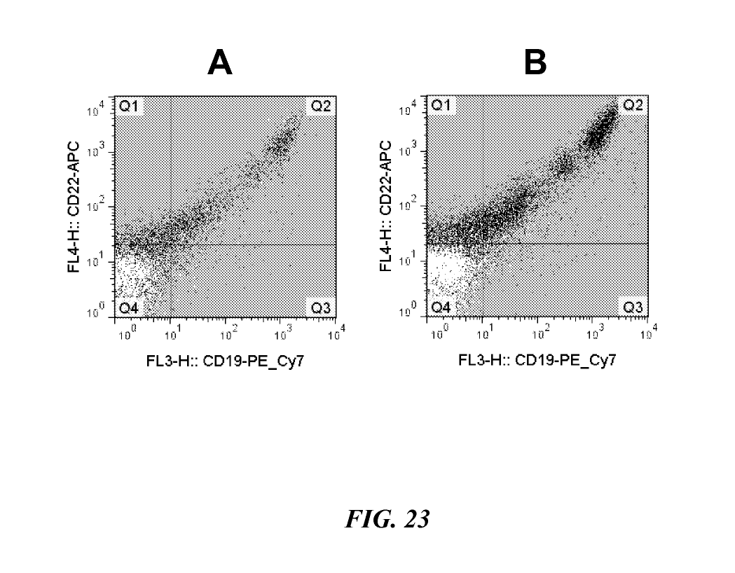

CD19 and CD22 were significantly reduced from Daudi cells in monocyte-depleted PBMCs (FIG. 21), suggesting the involvement of effector cells in addition to monocytes. NK cells, which express FcγRIII (CD16), are identified among PBMCs by flow cytometry as CD14−CD16+ cells located in the lymphocyte (forward vs. side scatter) gate. Using the Daudi/PBMC and Daudi/monocyte-depleted PBMC mixtures from Example 7, the lymphocyte gate was further gated for CD14 and CD16 to identify CD14⁻ CD16⁺ NK cells (FIG. 22). NK cells potently acquired CD19 and CD22 when either PBMCs (FIG. 23A) or monocyte-depleted PBMCs (FIG. 23B) were mixed with Daudi and epratuzumab. These results indicate that NK cells can function in epratuzumab-induced trogocytosis.

### Example 9

### Epratuzumab-Induced Trogocytosis with Granulocytes

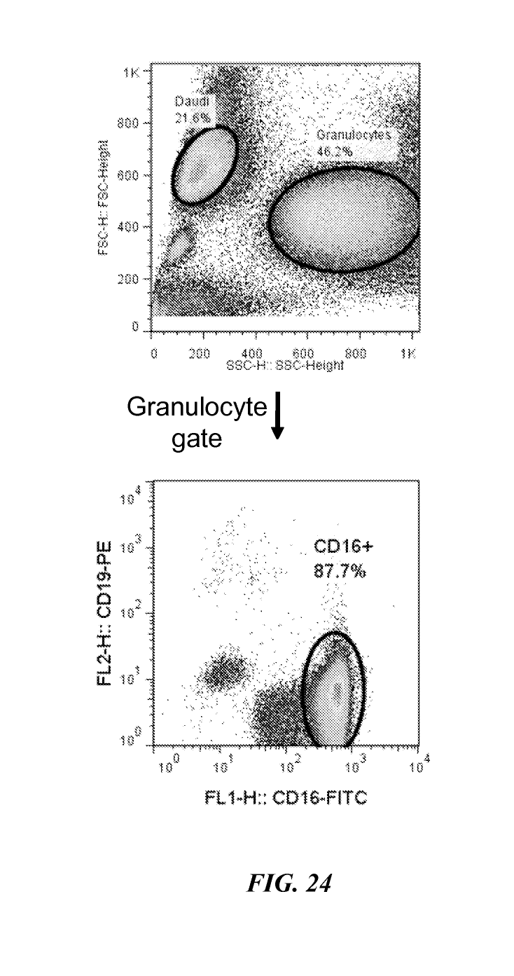

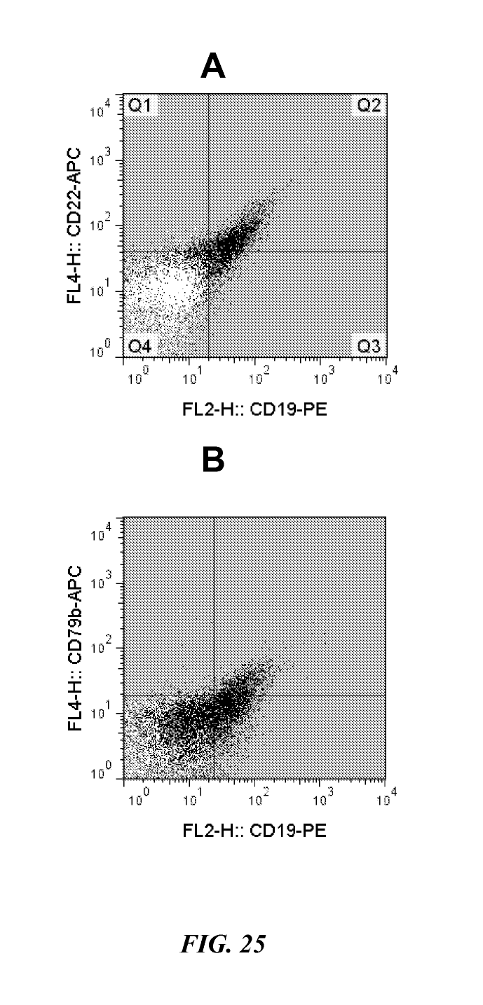

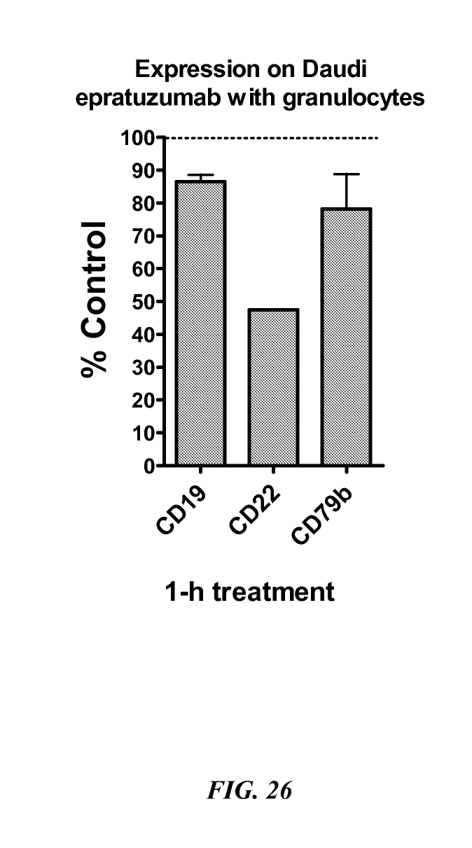

Granulocytes, or polymorphonuclear cells, which comprise mostly neutrophils, are separated from the PBMCs during processing of whole blood. Granulocytes, which express FcγRIII (CD16), were assessed for their ability to participate in trogocytosis when mixed with Daudi cells and epratuzumab. Granulocytes were readily gated from the Daudi cells by side scattering and CD16 (FIG. 24). When mixed with Daudi cells and treated with epratuzumab, but not the isotype control mAb, granulocytes stained positive for CD22 (30.4% positive), CD19 (40.9% positive) and CD79b (13.7% positive) (FIG. 25). Following the 1-h incubation, a significant reduction on Daudi of each antigen indicates their transfer from Daudi to granulocytes (FIG. 26).

TABLE 7
Trogocytosis of CD19 and CD22 from Daudi to monocytes, NK cells and
granulocytes following treatment with epratuzumab.

| Cells | mAb | % CD19⁺ | % CD22⁺ | % CD19⁺CD22⁺ |
|---|---|---|---|---|
| All | epratuzumab | 52.4 | 56.6 | 44.4 |
| Monocytes | hMN-14 | 10.1 | 5.3 | 1.6 |
| CD14⁺CD16⁺ | epratuzumab | 67.5 | 81.6 | 66.4 |
| Monocytes | hMN-14 | 4.3 | 6.7 | 2.3 |
| CD14⁺⁺ | epratuzumab | 35.4 | 48.9 | 31.4 |
| Monocytes | hMN-14 | 2.1 | 2.6 | 0.5 |
| CD14⁻CD16⁺ | epratuzumab | 46.3 | 58.0 | 43.6 |
| NK | hMN-14 | 3.7 | 4.7 | 2.4 |
| Granulocytes | epratuzumab | 40.9 | 30.4 | 26.8 |
| | hMN-14 | 2.2 | 1.9 | 0.5 |

Purified monocytes, monocyte-depleted PBMCs (CD14⁻CD16⁺ NK cells), or granulocytes were mixed with an equal number of Daudi cells and treated with 10 μg/mL epratuzumab or hMN-14 (anti-CEA mAb as control) for 1 h.

### Example 10

### Ex Vivo Trogocytosis with SLE Patient PBMCs

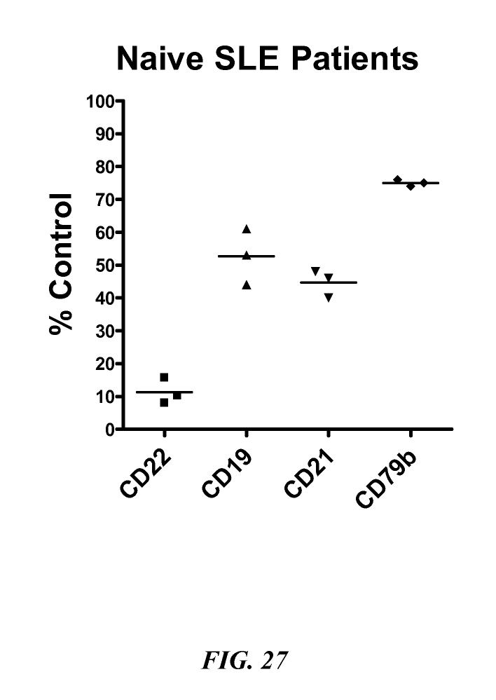

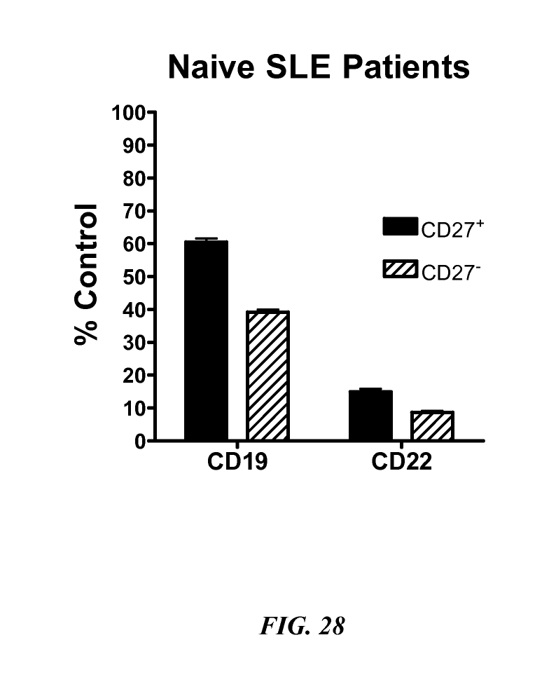

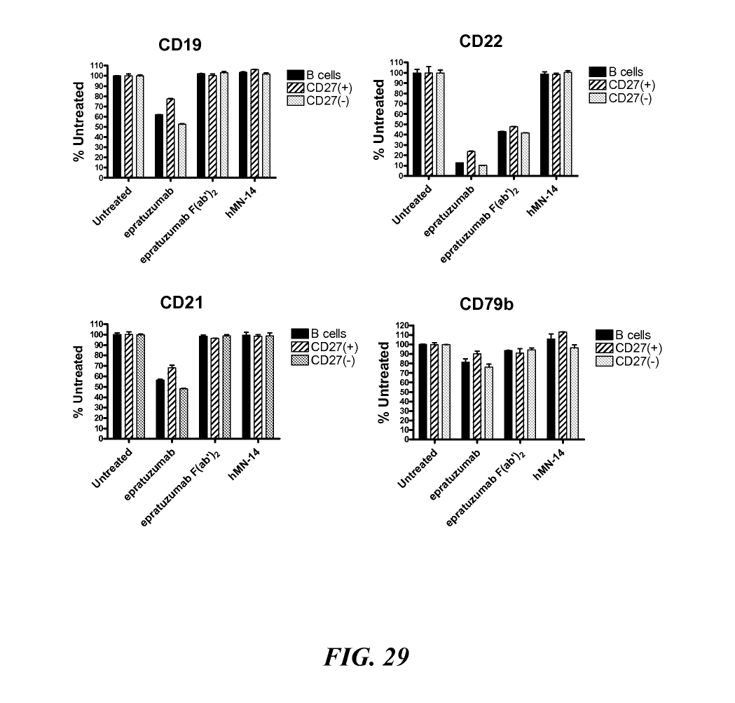

PBMCs were isolated from blood specimens of systemic lupus erythematosus (SLE, lupus) patients, who had yet to receive any therapy for their disease (naïve), and treated ex vivo with epratuzumab, using the same method that was applied to PBMCs from healthy donors. PBMCs of naive SLE patients responded similarly to healthy PBMCs (as in Example 1), where CD22, CD19, CD21 and CD79b on the surface of B cells were reduced to 11±4, 53±8, 45±4 and 75±1% control, respectively (FIG. 27). Also similar to the results from normal donor PBMCs, CD27⁻ naive B cells were more responsive than CD27⁺ memory B cells (FIG. 28), and, a F(ab′)₂ fragment of epratuzumab did not induce the reduction of CD19, CD21 or CD79b (FIG. 29). PBMCs isolated from blood specimens of SLE patients who currently were on epratuzumab immunotherapy had minimal response to the ex vivo treatment with epratuzumab (not shown), presumably due to low levels of CD22 on their B cells, resulting from therapy.

### Example 11

### Surface Levels of CD19, CD21, CD22 and CD79b on SLE Patient B Cells on Epratuzumab Immunotherapy

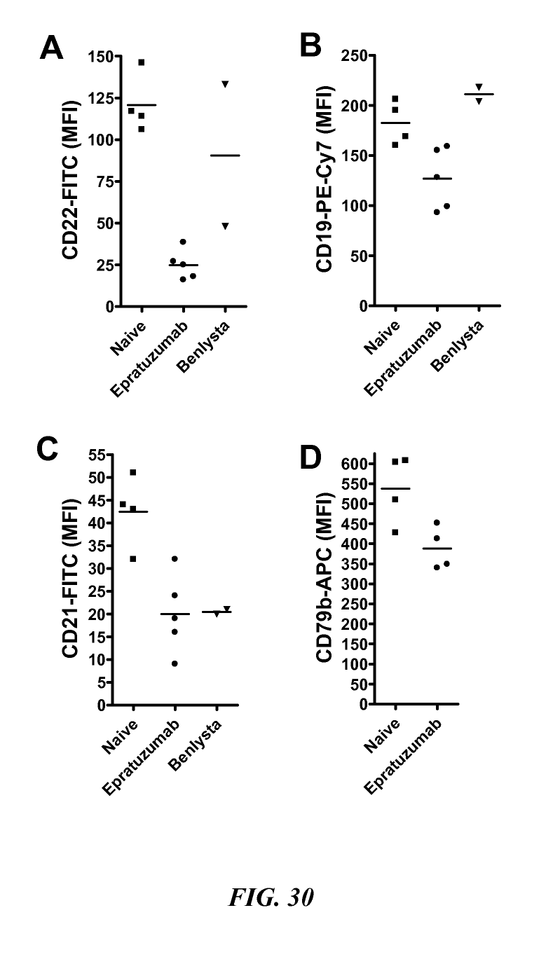

The relative levels of CD22, CD19, CD21 and CD79b on B cells from five SLE patients who were receiving epratuzumab immunotherapy, were compared the results obtained from four naive lupus patients and two receiving BENLYSTA®, using identical conditions (Table 8). Only one of the epratuzumab group (S7) had a markedly reduced B cell count; however, this patient was also taking prednisone and methotrexate. Each of the four patients on epratuzumab without methotrexate had B cell counts in the same range as the naive patients. Both BENLYSTA® patients had low B cell counts. As expected, CD22 was significantly (P<0.0001) lower (>80%) on the B cells of epratuzumab-treated patients (FIG. 30A). Notably, CD19, CD21 and CD79b were each significantly (P<0.02) lower for the epratuzumab group (FIG. 30B-D). We also compared the results for the epratuzumab patient specimens with those of two patients who were receiving immunotherapy with BENLYSTA®. Although the sample size is small, both CD19 and CD22 levels were significantly (P<0.05) lower on the B cells of patients on epratuzumab compared to BENLYSTA®. The level of CD21 was similarly low for the epratuzumab and BENLYSTA® patient B cells. Because anti-CD79b-PE (instead of APC) was used to measure CD79b on B cells from BENLYSTA® patients, we could only compare these results with one epratuzumab patient specimen, which was measured similarly. The CD79-PE MFI was greater for each of the BENLYSTA® specimens (MFI=425 and 470) compared to that of the epratuzumab sample (MFI=186).

TABLE 8
Comparison of B cells from lupus patients

| | | % B cell | | | | |
|---|---|---|---|---|---|---|
| | Treat- | in lymph- | CD19 | CD21 | CD22 | CD79b |
| Patient | ment | gate | (PE-Cy7) | (FITC) | (FITC) | (APC) |
| S7 | E, P, M | 0.5 | 99 | 9 | 16 | 186^{PE} |
| S8 | P, I | 5.0 | 145 | nd | 84 | nd |
| S9 | B | 0.5 | 218 | 21 | 48 | 470^{PE} |
| S10 | B | 0.9 | 204 | 20 | 133 | 425^{PE} |
| S11 | None | 18.0 | 195 | 51 | 106 | 608 |
| S12 | None | 13.1 | 160 | 44 | 114 | 428 |
| S13 | None | 13.3 | 206 | 43 | 117 | 510 |
| S14 | None | 11.1 | 169 | 32 | 146 | 604 |
| S16 | E, P | 8.9 | 128 | 24 | 27 | 452 |
| S17 | E, P | 4.5 | 93 | 16 | 25 | 340 |
| S18 | E, P | 17.6 | 159 | 32 | 18 | 413 |
| S19 | E, P | 20.3 | 155 | 19 | 38 | 349 |

E, epratuzumab;
P, prednisone;
M, methotrexate;
I, Imuran;
B, BENLYSTA ®;
^{PE}used instead of APC;
nd, not determined

The present studies disclose previously unknown, and potentially important, mechanisms of action of epratuzumab in normal and lupus B cells, as well as B-cell lymphomas, which may be more pertinent to the therapeutic effects of epratuzumab in autoimmune patients. The prominent loss of CD19, CD21, CD20, and CD79b induced by epratuzumab is not only Fc-dependent, but also requires further engagement with FcγR-expressing effector cells present in PBMCs. The findings of reduced levels of CD19 are of particular relevance for the efficacy of epratuzumab in autoimmune diseases, because elevated CD19 has been correlated with susceptibility to SLE in animal models as well as in patients, and loss of CD19 would attenuate activation of B cells by raising the BCR signaling threshold. Based on these findings, the activity of epratuzumab on B cells is two-fold, one via binding to CD22, which also occurs with F(ab′)₂, and the other via engagement of FcγR-bearing effector cells. Whereas the former leads to internalization of CD22, as well as its phosphorylation with concurrent relocation to lipid rafts (resulting in the activation of tyrosine phosphatase to inhibit the activity of Syk and PLCr2), the latter results in the trogocytosis (shaving) of CD19, among others.

We propose that the consequences of losing CD19 from B cells are as follows. BCR activation upon encountering membrane-bound antigen involves the initial spreading and the subsequent formation of microclusters. Because CD19 is critical for mediating B-cell spreading, CD19-deficient B cells are unable to gather sufficient antigen to trigger B-cell activation. In addition, loss of CD19 on B cells may severely affect the ability of B cells to become activated in response to T cell-dependent antigens. Thus, the epratuzumab-mediated loss of CD19 (and possibly other BCR markers and cell-adhesion molecules) on target B cells may incapacitate such B cells and render them unresponsive to activation by T cell-dependent antigen. In summary, epratuzumab inactivates B cells via the loss of CD19, other BCR constituents, and cell-adhesion molecules that are involved in sustaining B-cell survival, leading to therapeutic control in B-cell-mediated autoimmune diseases. Although targeting B cells with either epratuzumab to CD22 or rituximab to CD20 appears to share a common effect of reducing CD19 by trogocytosis, we are currently investigating whether rituximab has a scope of trogocytosis as broad as epratuzumab. The results also caution that using CD19 as a marker for quantifying B cells by flow cytometry from patients treated with agents that induce CD19 trogocytosis may result in an over-estimation of B-cell depletion.

It has been shown with rituximab administered to chronic lymphocytic leukemia cells that too much antibody results in removal of complexes of rituximab-CD20 from the leukemia cells by trogocytosis to monocytes, and can enable these malignant cells to escape the effects of the antibody by antigenic modulation. It was then found that reducing the dose of therapeutic antibody could limit the extent of trogocytosis and improve the therapeutic effects (Herrera et al., 2006). Based on our present findings, a similar process of antigen shaving (trogocytosis) by anti-CD22 or anti-CD20 antibodies that extends beyond the respective targeted antigens can be implicated in the therapy with epratuzumab or rituximab (or the humanized anti-CD20 mAb, veltuzumab). This could explain the clinical observations that higher doses of epratuzumab administered to SLE or lymphoma patients did not show an improvement in efficacy over the mid-range dose used, because the concentrations of epratuzumab in serum would be in the μM range (150 μg/mL or higher) and could mask the low-affinity FcγRs on effector cells, thus reducing the likely events of trogocytosis.

### Example 12

### Time Course of Trogocytosis

The events of epratuzumab-induced trogocytosis were studied by flow cytometry over 20 h using Daudi cells and purified monocytes. Within 15 min, >50% of the monocytes were bound to Daudi cells, due to the formation of epratuzumab-mediated immunological synapses; and, nearly half of the remaining (unconjugated) monocytes (FSClow/CD20−) were already CD22 and CD19 positive (not shown). The Daudi/monocyte conjugates (CD14+CD20+CD19++FSChigh) dissociated rapidly (not shown). Although the presence of Daudi cells prohibited measurement of CD22/CD19 transfer to monocytes among the conjugates, the levels of CD22 and CD19 in the unconjugated monocytes peaked before 30 min and returned to baseline by 20 h (not shown), presumably due to internalization. The levels of CD22 and CD19 on unconjugated Daudi (CD14−CD20+FSClow) decreased sharply over the first 30 min, and then continued to decline more gradually throughout the incubation (not shown). The isotype control did not alter the levels of either marker over the duration of the experiment.

### Example 13

### Administration Of Epratuzumab in Systemic Lupus Erythematosus (SLE)

An open-label, single-center study of patients with moderately active SLE (total British Isles Lupus Assessment Group (BILAG) score 6 to 12) is conducted. Patients receive dosages of epratuzumab of 100, 200, 400 and 600 mg subcutaneously (SC) every week for 6 weeks. Evaluations include safety, SLE activity (BILAG), blood levels of B and T cells, human anti-epratuzumab antibody (HAHA) titers, and levels of cell surface CD19, CD20, CD21, CD22 and CD79b on B cells. It is determined that a dosage of 400-600 mg per SC injection results in optimal depletion of B cell CD19, while producing less than 50% depletion of normal B cells. Subsequently, a subcutaneous dose of 400 mg epratuzumab is administered to a new group of patients with moderately active SLE.

Total BILAG scores decrease by at least 50% in all patients, with 92% having decreases continuing to at least 18 weeks. Almost all patients (93%) experience improvement in at least one BILAG B- or C-level disease activity at 6, 10 and 18 weeks. Additionally, 3 patients with multiple BILAG B involvement at baseline have completely resolved all B-level disease activities by 18 weeks. Epratuzumab is well tolerated, with no evidence of immunogenicity or significant changes in T cells, immunoglobulins or autoantibody levels. B-cell levels decrease by an average of 35% at 18 weeks and remain depressed for 6 months post-treatment.

### Example 14

### Prediction Of Epratuzumab Response in Systemic Lupus Erythematosus (SLE)

Another open-label, single-center study of patients with moderately active SLE is conducted. Patients receive a single dose of 400 mg epratuzumab subcutaneously. Blood levels of B and T-cells and levels of cell surface CD19, CD20, CD21, CD22 and CD79b on B cells are determined.

Patients are divided into two groups, based on whether they show a decrease in B-cell CD19 levels above (responders) or below (non-responders) the median response for the group. It is observed that decreased B-cell CD19 levels are correlated with decreases in B-cell CD20, CD21, CD22 and CD79b. Subsequent s.c. administration of 400 mg of epratuzumab occurs every week for 8 weeks and SLE activity (BILAG) is monitored.

The group of responders shows a substantial improvement in BILAG scores compared with the group of non-responders. Three of ten patients in the responders group have completely resolved all BILAG B-level disease activities by 18 weeks, compared with zero of ten patients in the non-responders group. In addition, a significant improvement in total BILAG scores is observed in the responders group compared to the non-responders. It is concluded that trogocytosis (antigen-shaving) of CD19 and other BCR antigens is predictive of therapeutic response to therapy with anti-CD22 antibody in SLE.

### Example 15

### Administration Of Epratuzumab in Hairy Cell Leukemia

Patients with previously untreated or relapsed hairy cell leukemia receive 4 doses of 80, 160, 320 or 640 mg epratuzumab injected s.c. every week or every two weeks. Occasional mild to moderate transient injection reactions are seen with the s.c. injection and no other safety issues are observed. The s.c. epratuzumab exhibits a slow release pattern over several days. Transient B-cell depletion is observed at all dosage levels of epratuzumab. Depletion of B cell surface levels of CD19, CD20, CD21, CD22 and CD79b is observed at a moderate level with 320 mg and at a much higher level at 640 mg epratuzumab.

Objective responses are observed at all dose levels of s.c. epratuzumab, but with particularly high responses of 30% (mostly partial responses) at the dose of 320 mg. All serum samples evaluated for human anti-epratuzumab antibody (HAHA) are negative. Six months after treatment, optimal outcome is observed in the group treated with 320 mg epratuzumab, with decreased response at either higher or lower dosages. It is concluded that under these conditions, 320 mg epratuzumab is the optimum dosage that was used. Monitoring response of BCR levels to therapeutic antibody provides an effective surrogate marker for determining antibody efficacy and is predictive of disease prognosis in response to therapy.

### Example 16

### Trogocytosis of BCR-Response Modulating Proteins Induced by the RFB4 Anti-CD22 Antibody

Trogocytosis of BCR-regulating proteins, including CD19, CD21, CD22 and CD79b is assayed as described in Example 1 in response to exposure to the anti-CD22 antibody RFB4, which binds to a different epitope of CD22 than epratuzumab. Control antibody (hMN-14) is used as described in Example 1. Exposure to RFB4 antibody induces trogocytosis of BCR-regulating proteins, similar to that induced by epratuzumab as disclosed in Example 1. CD27⁻ naive B cells are more responsive to RFB4 compared to CD27⁺ memory B cells. The effect is essentially complete within a few hours. The reductions in surface CD19 and CD21 are not significantly different following 2-h or overnight treatment.

### Example 17

### Mechanism Of Cytotoxicity Induced on Malignant B Cells by Anti-CD22 Antibody (Epratuzumab)

Summary

Epratuzumab has shown activity in patients with non-Hodgkin lymphoma, systemic lupus erythematosus, and Sjögren's syndrome, but the mechanism by which it depletes B cells in vivo has previously been unknown. In vitro, epratuzumab is cytotoxic to CD22-expressing human Burkitt lymphoma lines only when immobilized onto plastic plates or combined with a secondary antibody plus anti-IgM.

We used a Daudi lymphoma subclone selected for high expression of membrane IgM (mIgM) to investigate the cytotoxic mechanism of immobilized epratuzumab, and showed that it induced similar intracellular changes as observed upon crosslinking mIgM with anti-IgM. Specifically, we identified phosphorylation of CD22, CD79a and CD79b, and their translocation to lipid rafts, as essential for cell killing. Other findings include the co-localization of CD22 with mIgM, forming caps before internalization; induction of caspase-dependent apoptosis (25-60%); and a pronounced increase of pLyn, pERKs and pJNKs with a concurrent decrease of constitutively-active p38. The apoptosis was preventable by JNK or caspase inhibitors, and involved mitochondrial membrane depolarization, generation of reactive oxygen species, upregulation of pro-apoptotic Bax, and downregulation of anti-apoptotic Bcl-x1, Mcl-1 and Bcl-2. These findings indicated, for the first time, that epratuzumab and anti-IgM behave similarly in perturbing multiple BCR-mediated signals in malignant B cells.

Introduction

Epratuzumab (hLL2), a humanized anti-CD22 monoclonal antibody, is currently under clinical investigation for the treatment of non-Hodgkin lymphoma (NHL) and systemic lupus erythematosus (SLE). CD22, also referred to as sialic acid-binding Ig-like lectin-2 (Siglec-2) or B-lymphocyte adhesion molecule (BL-CAM), is a transmembrane type-I glycoprotein of 140 kDa, widely and differentially expressed on B cells (Kelm et al., 1994, Curr Biol 4:965-972; Law et al., 1995, J Immunol 155:3368-76; Wilson et al., 1991, J Exp Med 173:137-46). Structurally, the extracellular portion of CD22 comprises 7 Ig-like domains, of which the two N-terminal domains are involved in ligand binding, while the cytoplasmic tail contains 6 conserved tyrosine residues localized within the immunoreceptor tyrosine-based inhibition motifs (ITIM) and immunoreceptor tyrosine-based activation motifs (ITAM) (Wilson et al., 1991, J Exp Med 173:137-46; Schulte et al., 1992, Science 258:1001-4; Torres et al., 1992, J Immunol 149:2641-49). Functionally, CD22 recognizes α2,6-linked sialic acids on glycoproteins in both cis (on the same cell) and trans (on different cells) locations, and modulates B cells via interaction with CD79a and CD79b, the signaling components of the B-cell receptor (BCR) complex (Leprince et al., 1993, Proc Natl Acad Sci USA 90:3236-40; Peaker et al., 1993, Eur J Immunol 23:1358-63). Crosslinking BCR with cognate antigens or appropriate antibodies against membrane immunoglobulin (mIg) on the cell surface induces translocation of the aggregated BCR complex to lipid rafts, where CD79a, CD79b and CD22, among others, are phosphorylated by Lyn (Marshall et al., 2000, Immunol Rev 176:30-46; Niiro et al., 2002, Nat Rev Immunol 2:945-56; Smith et al., 1998, J Exp Med 187:807-11), which in turn triggers various downstream signaling pathways, culminating in proliferation, survival, or death (Peaker et al., 1993, Eur J Immunol 23:1358-63; Niiro et al., 2002, Nat Rev Immunol 2:945-56; Pierce & Liu, 2010, Nat Rev Immunol 10:767-77). Importantly, phosphorylated CD22, depending on environmental cues, can either positively or negatively affect BCR-mediated signaling pathways (Niiro et al., 2002, Nat Rev Immunol 2:945-56; Pierce & Liu, 2010, Nat Rev Immunol 10:767-77; Nitschke, 2005, Curr Opin Immunol 17:290-97; Otipoby et al., 2001, J Biol Chem 276:44315-22). Understanding the role of CD22 in B-cell malignancies, as well as B-cell-implicated autoimmune diseases, is of considerable interest.

As a single agent, epratuzumab is well-tolerated and depletes circulating B cells in patients with NHL, SLE, and Sjögren's syndrome by 35 to 50% (Goldenberg, 2006, Expert Rev Anticancer Ther 6:1341-53; Leonard & Goldenberg, 2007, Oncogene 26:3704-13; Leonard et al., 2003, J Clin Oncol 21:3051-59; Leonard et al., 2004, Clin Cancer Res 10:5327-34; Dorner et al., 2006, Arthritis Res Ther 8:R74). It has modest antibody-dependent cellular cytotoxicity (ADCC) but no complement-dependent cytotoxicity in vitro (Carnahan et al., 2007, Mol Immunol 44:1331-41). In vivo, it targets CD27⁻ naïve and transitional B cells, and decreases surface CD22 expression (Jacobi et al., 2008, Ann Rheum Dis 67:450-57). Epratuzumab downregulates the surface expression of certain adhesion molecules (CD62L and (37 integrin), and increases the expression of β1 integrin on CD27⁻ B cells, resulting in migration of B cells towards the chemokine, CXCL12 (Daridon et al., 2010, Arthritis Res Ther 12:R204). Soluble epratuzumab does not have cytotoxic or cytostatic effects in vitro or in xenografts of human lymphoma in vivo (Carnahan et al., 2007, Mol Immunol 44:1331-41; Carnahan et al., 2003, Clin Cancer Res 9:3928 S-90S; Stein et al., 1993, Cancer Immunol Immunother 37:293-98). However, when immobilized to plastic plates or added in combination with suboptimal amounts of anti-IgM along with a crosslinking secondary antibody, it induces growth-inhibition in NHL cell lines, such as Ramos and Daudi (D1-1), a subclone of Daudi selected for a high expression of BCR (Qu et al., 2008, Blood 111:2211-19). We have reported previously that soluble epratuzumab phosphorylates and translocates CD22 to lipid rafts upon engagement (Qu et al., 2008, Blood 111:2211-19), but the exact mechanism by which epratuzumab kills normal and malignant B cells in patients, and inhibits the growth of lymphoma lines in vitro upon immobilization, remains elusive.

In this study, we evaluated key signaling pathways and molecules affected by immobilized epratuzumab. We showed in D1-1 cells that epratuzumab by either non-covalent adsorption on microtiter plates or conjugated covalently to polystyrene beads induces phosphorylation of CD22, CD79a and CD79b, and their translocation to lipid rafts, which are instrumental for cell death via caspase-dependent apoptosis. Additional experiments showed that immobilization of epratuzumab also induces substantial apoptosis (25 to 60%) in Ramos lymphomas. A pronounced phosphorylation of ERK and JNK MAP kinases, accompanied by a decrease in phosphorylated p38 MAP kinase, also was observed. Selective experiments interrogating intracellular events identified changes in mitochondrial membrane potential, generation of reactive oxygen species (ROS), involvement of caspases, and modulation of pro- and anti-apoptotic proteins, in the mechanisms of immobilized epratuzumab.

Materials and Methods

Cell Lines, Antibodies, and Reagents—

The Burkitt lymphoma cell lines, Daudi and Ramos, were obtained from ATCC (Manassas, Va.). D1-1, a subclone of Daudi selected for a higher expression of the BCR, was developed in-house (Qu et al. 2008, Blood 111:2211-19). Phospho-specific and other antibodies were obtained from CELL SIGNALING TECHNOLOGY®(Danvers, Mass.) and SANTA CRUZ BIOTECHNOLOGY® (Santa Cruz, Calif.). Anti-tyrosine antibody 4G10 was bought from Millipore (Billerica, Mass.), anti-IgM antibody, secondary goat anti-human Fc specific and rhodamine conjugated F(ab′)₂ fragment goat anti-human IgG, F(ab′)₂ fragment specific were obtained from Jackson ImmunoResearch (West Grove, Pa.). Cell culture media, supplements, annexin V ALEXA FLUOR® 488 conjugate, TMRE, and CM-H₂DCF-DA were supplied by INVITROGEN™ (Grand Island, N.Y.). One Solution Cell Proliferation assay reagent was obtained from Promega (Madison, Wis.). PHOSPHOSAFE™ and RIPA buffers were procured from EMD chemicals (Billerica, Mass.). For epratuzumab immobilization, non-tissue-culture flat-bottom polystyrene plates were obtained from BD Biosciences (San Jose, Calif.), and CP-30-10 carboxyl-coated polystyrene beads were bought from Spherotech (Lake Forest, Ill.). All other chemicals were obtained from SIGMA-ALDRICH® (St. Louis, Mo.).

Immobilization of Epratuzumab—

Epratuzumab (10 μg/mL or as indicated) in carbonate/bicarbonate buffer (50 mM; pH 9.6) was immobilized on non-tissue-culture flat-bottom plates by incubating the plate at 4° C. overnight. Next day, plates were washed 2× with RPMI-1640 medium. Besides immobilizing epratuzumab onto plates, 100 μg was also immobilized to Protein A beads (100 μL). Supernatants were analyzed for the amounts of epratuzumab bound to the beads. Epratuzumab-bound beads were washed 3× with PBS and reconstituted in 100 μL of the RPMI-1640 medium. For flow cytometry, epratuzumab also was conjugated to CP-30-10 carboxyl-coated polystyrene beads using the manufacturer's protocol. Briefly, 50 μg of epratuzumab was conjugated to 200 μL of polystyrene beads in 1 mL of MES buffer containing 20 mg of EDC for 30 min. Beads were washed 3× with PBS and reconstituted in 0.05M MES buffer containing 0.05% BSA.

Cell Culture and Cytotoxicity Assay—

Cell lines were cultured in RPMI-1640 medium supplemented with 10% heat-inactivated fetal bovine serum (FBS), 2 mM L-glutamine, 200 U/mL penicillin, and 100 μg/mL streptomycin in a humidified incubator at 37° C. with 5% CO₂. To evaluate the functional activity of epratuzumab or epratuzumab F(ab′)₂, different amounts (5, 10 and 20 μg/mL) were immobilized in 48-well plates. Plates were washed and D1-1 or Ramos cells were seeded (1×10⁴ cells per well) and incubated for 4 days. The number of viable cells was then determined using the MTS assay per the manufacturer's protocol, plotted as percent of the untreated. Activity of soluble epratuzumab or epratuzumab F(ab′)₂ was also evaluated.

Annexin V Binding Assay—

Cells in 6-well plates (2×10⁵ cells per well) were either treated with epratuzumab immobilized to polystyrene beads or immobilized to plates for 24 or 48 h, washed, resuspended in 100 μl of annexin-binding buffer, and stained with 5 μl of Annexin V-ALEXA FLUOR® 488 conjugate for 20 min. Cells were then stained with 1 μg/mL propidium iodide (PI) in 400 μl of annexin-binding buffer, and analyzed by flow cytometry (FACSCALIBUR™). When required, cells were pretreated with the indicated inhibitors for 2 h before adding the test article.

Immunoblot Analysis—

D1-1 and Ramos cells (2×10⁷ cells) were added to plates immobilized with epratuzumab (10 μg/mL) for varying time points as indicated. Cells were washed with PBS, lysed in ice-cold PHOSPHOSAFE™ buffer, and the lysates clarified by centrifugation at 13,000×g. Protein samples (25 μg/lane) were resolved by SDS-PAGE on 4-20% gradient tris-glycine gels followed by transfer onto nitrocellulose membranes.

Isolation of Lipid Rafts—

D1-1 cells (3×10⁷) were treated with the indicated antibodies or added to plates coated with epratuzumab (10 μg/mL) for 2 h. After treatment, cells were lysed in 2 mL of buffer containing CHAPS/low-salt (20 mM NaCl and 40% sucrose), and lysates were fractionated in a sucrose gradient and lipid rafts were prepared as described earlier (Qu et al., 2008, Blood 111:2211-19).

Co-Immunoprecipitation Analysis—

Six-well plates were coated with the required antibodies (10 μg/mL) in carbonate/bicarbonate buffer for 24 h. Plates were washed with RPMI-1640 medium containing 5% FBS, and D1-1 cells were added to the wells (5×10⁶ cell/well) for 2 h. Following incubation, cells were lysed in ice-cold RIPA buffer, and co-immunuprecipitation was performed using phospho-tyrosine antibody (4G10; 1:200 dilution), as described earlier (Gupta et al., 2006, Cancer Res 66:8182-91). 20 μl of the samples were separated by SDS-PAGE and transferred onto a nitro-cellulose membrane, followed by probing with the indicated antibodies.

Mitochondrial Membrane Potential (Δψₘ) and Reactive Oxygen Species (ROS) Assays—

D1-1 cells (2×10⁵ cells per well) were added to the 6-well plates coated with epratuzumab (10 μg/mL) for 48 h. Cells were washed and stained for 30 min in the dark at 37° C., either with TMRE (50 nM) for Δψₘ analysis or CM-H₂DCF-DA (1 μM) for ROS analysis. Samples were washed 3× with PBS and analyzed for changes in fluorescence using flow cytometry.

Immunofluorescence Analysis—

To analyze the co-localization of CD22 and IgM receptors, D1-1 cells were treated with epratuzumab (7.5 μg/mL) or anti-IgM conjugated to ALEXA FLUOR® 488 (1 μg/mL) alone and in combination with a secondary crosslinking goat anti-human antibody for 5 min at 37° C. Cells were washed with PBS to remove the antibodies and incubated at room temperature for 30 min, followed by fixation with 4% paraformaldehyde, and staining with rhodamine-conjugated Fc-specific goat anti-human IgG for 20 min. Cells were washed with PBS and visualized by fluorescence microscopy. To evaluate the translocation of CD22 in lipid rafts, D1-1 cells were incubated with Protein A-immobilized epratuzumab for 4 h, fixed, and permeabilized with 0.1% Triton X-100 in PBS. CD22 and IgM receptors were evaluated by epratuzumab-dylight 550 and anti-IgM-ALEXA FLUOR® 488, respectively. Images were overlaid using Photoshop software.

Cell Cycle Analysis—

Cells were seeded in 6-well plates (2×10⁵ cells per well) and treated with epratuzumab conjugated to polystyrene beads or the indicated antibodies for 72 h. Following incubation, cell cycle analysis was performed by flow cytometry as described (Gupta et al., 2010, Blood 116:3258-67).

Results

Immobilization of epratuzumab induces growth-inhibition and apoptosis—

The ability to induce growth-inhibition was evaluated by immobilizing epratuzumab to non-tissue-culture coated flat-bottom plates. Varying amounts of epratuzumab were immobilized. In the cell viability assay, 5 μg/mL of immobilized epratuzumab induced significant growth-inhibition in D1-1 cells (data not shown). About 60% growth-inhibition was observed at this concentration, and little change was found at higher concentrations of 10 and 20 μg/mL, indicating saturation (data not shown). Similar growth-inhibition of the Burkitt lymphoma line, Ramos, was observed, although it was slightly less than with D1-1. In Ramos cells, 10 μg/mL epratuzumab induced about 45% growth-inhibition (data not shown). This difference in sensitivity could be due to the levels of CD22 and overexpression of BCR components in D1-1. Immobilized nonspecific hMN-14 antibody did not induce growth-inhibition in either cell line (data not shown). Soluble epratuzumab in the media, even at the highest concentration (20 μg/mL), did not induce growth-inhibition in either cell line, indicating the requirement of immobilization (data not shown).

We next evaluated the role of apoptosis in the effect of epratuzumab. Carboxyl-coated polystyrene beads were used to immobilize epratuzumab. Exposure to 5 and 20 μL, of epratuzumab-coated beads induced apoptosis in both D1-1 and Ramos at 24 h (data not shown). In D1-1, 5 μL of epratuzumab-coated beads induced about 75% apoptosis, while similar amounts of uncoated beads displayed annexin V staining, comparable to untreated cells (data not shown). Significant apoptosis was also observed in Ramos cells by epratuzumab-coated beads (data not shown). Likewise, Protein A-immobilized epratuzumab induced apoptosis and growth-inhibition in both D1-1 and Ramos cells (data not shown). These results demonstrate the requirement of epratuzumab immobilization onto plastic or to beads for inducing growth-inhibition and apoptosis in the target malignant cells. Similar to epratuzumab, the immobilized F(ab′)₂ fragments of epratuzumab also induced apoptosis and growth inhibition in D1-1 cells (data not shown). These results negate the role of Fc effector functions and confirm the role of signaling events in the target cells for observed growth inhibition though immobilization.

Immobilized Epratuzumab Induces Phosphorylation of CD22, CD79a and CD79b—

To understand the mechanism by which immobilized epratuzumab inhibits growth in these lymphoma lines, we evaluated the phosphorylation profiles of the BCR components, CD79a and CD79b. CD79a and CD79b form hetrodimers and are noncovalently-bound membrane immunoglobulins that regulate BCR-mediated signaling by ITAM motifs in their cytoplasmic tails. Cells were subjected to immobilized epratuzumab and other antibodies for 2 h, and co-immunoprecipitation experiments were performed using the phospho-tyrosine antibody, 4G10. Anti-IgM (10 μg/mL) antibody induced phosphorylation of CD22, CD79a and CD79b molecules, while soluble epratuzumab induced phosphorylation of CD22, but not CD79a and CD79b (data not shown). Immobilization of anti-IgM and epratuzumab induced phosphorylation of CD22 as well as CD79a and CD79b (data not shown). Ligation of CD22 on D1-1 by immobilized epratuzumab was similar to ligation of BCR by anti-IgM (above a threshold concentration, i.e., 10 μg/mL), in that both resulted in the phosphorylation of CD22, CD79a and CD79b. Similar phosphorylation of CD22, CD79a and CD79b was observed with soluble epratuzumab combined with suboptimal amounts of anti-IgM (1 μg/mL) and a secondary crosslinking goat anti-human IgG, while anti-IgM (1 μg/mL) alone did not induce phosphorylation of any of these molecules (data not shown). Soluble epratuzumab in combination with anti-IgM and a secondary crosslinking antibody has been observed previously to induce growth-inhibition in lymphoma lines. These results with respect to differences in the phosphorylation profiles of CD79a and CD79b by soluble and immobilized epratuzumab clearly implicate components of BCR in the growth-inhibition due to immobilized epratuzumab or the combination of epratuzumab and anti-IgM antibody.

Immobilized Epratuzumab Translocates CD22 and CD79 to Lipid Rafts—

The observation that immobilized epratuzumab induces phosphorylation of BCR components, CD79a and CD79b, prompted us to investigate the membrane distribution of CD22, CD79a and CD79b in lipid rafts, using sucrose density gradient ultracentrifugation. Anti-IgM (10 μg/mL) treatment resulted in the distribution of CD22, CD79a and Cd79b into lipid rafts (data not shown). Soluble epratuzumab, which is known to induce phosphorylation of CD22 and migration of CD22 into lipid rafts (Qu et al., 2008, Blood 111:2211-19), did not induce redistribution of CD79a and CD79b into lipid rafts (data not shown). However, soluble epratuzumab together with suboptimal amounts of anti-IgM (1 μg/mL) and a secondary crosslinker resulted in the migration of CD22, CD79a and CD79b into lipid rafts. Since soluble epratuzumab together with anti-IgM (1 μg/mL) and a crosslinker induced growth-inhibition in these malignant cells, the presence of phosphorylated CD22, CD79a and CD79b in lipid rafts seems to be critical for the effects of epratuzumab. Immobilized epratuzumab also induced migration of these components into lipid rafts, although the signals were not as strong as they were for other samples; this could be due to loss of some treated cells because of adherence to the epratuzumab-coated plates (data not shown).

We also examined the distribution of CD22 and BCR components by immunofluorescence. Soluble epratuzumab binds to CD22 and internalizes rapidly into the cells (Carnahan et al., 2003, Clin Cancer Res 9:3982 S-90S). To study the distribution of CD22 and IgM receptors, we treated the cells with different antibodies alone or in combination for 5 min at 37° C. Cells were fixed after 30 min. Immunofluorescence analysis revealed the binding of soluble epratuzumab and anti-IgM to cell-surface CD22 and IgM receptors, respectively, when the two antibodies were evaluated separately (data not shown). However, when soluble epratuzumab combined with suboptimal amounts of anti-IgM (1 μg/mL) were added, they formed caps and co-localized in about 70% of cells (data not shown). Similar co-localization of CD22 and IgM receptors was observed when cells were treated with Protein A-bound epratuzumab (data not shown). These observations indicate the co-localization and requirement of both IgM and CD22 receptors, either when soluble epratuzumab is used together with suboptimal amounts of anti-IgM or when epratuzumab is immobilized.

Requirement of Lyn for Growth-Inhibition by Immobilized Epratuzumab—

Lyn plays a critical role in regulating BCR activity by phosphorylating tyrosine residues in the ITAM domain of CD79a, CD79b, and ITIM domain in CD22, followed by recruitment of SHP-1 to CD22 (Schulte et al., 1992, Science 258:1001-4; Nitschke, 2005, Curr Opin Immunol 17:290-97; Chaouchi et al., 1995, J Immunol 154:3096-104; Doody et al., 1995, Science 269:242-44; Nitschke 2009, Immunol Rev 230:128-43). To understand this growth-inhibition, we evaluated the phosphorylation profiles of Lyn as a function of time. D1-1 cells were added to epratuzumab-coated plates for different times up to 4 h. Cells were lysed in RIPA buffer and phospho-tryosine residues were immunoprecipitated using monoclonal antibody 4G10. Immobilized epratuzumab induced rapid phosphorylation of tyrosine residues that continued for 4 h (not shown). Probing the same membranes with different antibodies depicted rapid and sustained phosphorylation of Lyn and Syk molecules (not shown). In a separate experiment, we repeated these studies until 24 h, and observed that immobilized epratuzumab induces the phosphorylation of Lyn and PLCγ2 (not shown). Although we observed phosphorylation of Syk by co-immunoprecipitation, we did not observe a similar time-dependent phosphorylation of Syk by using anti-phospho Syk antibodies (not shown).

To further elucidate the role of Lyn in this growth-inhibition by immobilized epratuzumab, we evaluated the binding of SHP-1 to the tyrosine residues. Cells were treated with various antibody combinations and a co-immunoprecipitation experiment was performed using antibody 4G10. Membranes were probed with SHP-1 antibody and the results indicate binding of SHP-1 to tyrosine residues in the samples treated with immobilized epratuzumab (not shown). Similar binding of SHP-1 was observed in samples treated with epratuzumab and suboptimal amounts of anti-IgM in presence of a secondary crosslinking antibody (not shown). In contrast, no significant binding was observed in samples treated with soluble epratuzumab or suboptimal amounts of anti-IgM alone (not shown). These results establish the requirement of phosphorylation of Lyn and recruitment of SHP-1 to CD22 to negatively regulate BCR signaling resulting in growth-inhibition.

Modulation Of MAP Kinases—

Mitogen-activated protein (MAP) kinases are a group of serine threonine protein kinases that respond to a variety of environmental cues, such as growth factors, cellular stress (e.g., UV, osmotic shock, DNA damage) and others, by either inducing survival and cell growth, or apoptosis. Previously, we observed that the anti-HLA-DR mAb, IMMU-114, induced growth-inhibition by hyperactivation of the ERK and JNK group of MAP kinases, while p38 was not affected (Stein et al., 2010, Blood 115:5180-90). To further elucidate the mechanism of growth-inhibition by immobilized epratuzumab, we studied the effects on all three MAP kinases. Immobilized epratuzumab induced modest activation and phosphorylation of the ERK and JNK group of MAP kinases (not shown). This activation was rapid, and could be detected within 30 min and sustained over a period of 24 h. In contrast, p38, the third group of MAP kinases, was inhibited and the phoshorylation of p38 was downregulated by immobilized epratuzumab within 30 min of treatment of the target cells (not shown).

We further studied the role of stress in the growth-inhibition by immobilized epratuzumab in the presence of an inhibitor of stress-activated JNK MAP kinase, SP600125. Two doses (2.5 and 5 nM) of the inhibitor were evaluated and at both doses, apoptosis was inhibited significantly in D1-1 cells (not shown). Thus, this differential activation/inhibition of MAP kinases attests to the fact that immobilized epratuzumab affects target cells by invoking multiple signaling pathways.

Immobilized Epratuzumab Induces Production of ROS and Changes in Mitochondrial Membrane Potential—

Induction of stress in cells results in the generation of free oxygen radicals in mitochondria. ROS are chemically-reactive oxygen molecules that induce mitochondrial membrane depolarization, activating pro-apoptotic proteins such as Bax, and resulting in programmed cell death in the target cells. To further investigate the role of stress in this growth-inhibition by immobilized epratuzumab, we studied the generation of ROS and changes in mitochondrial membrane potential in the affected cells. Treatment with immobilized epratuzumab resulted in about 24% cells having enhanced ROS production compared to about 10% in D1-1 cells treated with soluble epratuzumab or untreated (not shown).

Immobilized epratuzumab induced mitochondrial membrane depolarization in about 45% of D1-1 cells, compared to about 20% of cells treated with immobilized nonspecific hMN-14 antibody or untreated (not shown). Similar results for ROS and changes in mitochondrial membrane potential were observed in Ramos (data not shown).

Immobilized Epratuzumab Induces Caspase-Mediated Apoptosis—

We next evaluated the effect of immobilized epratuzumab on pro-/anti-apoptotic proteins and caspases in D1-1 and Ramos cells subjected to immobilized epratuzumab for 24, 48 and 72 h. Cell lysates were evaluated for the expression profiles of anti-apoptotic proteins, Bcl-2, Bcl-xL and Mcl-1, and pro-apoptotic protein, Bax. In both cell lines, immobilized epratuzumab downregulated anti-apoptotic proteins, Bcl-xL and Mcl-1, and increased the expression levels of pro-apototic, Bax (not shown). Bcl-2 was downregulated in D1-1, and very low levels were detected in Ramos. The observed apoptosis by immobilized epratuzumab in both D1-1 and Ramos was caspase-dependent, as observed by the cleavage of caspase 3, caspase 9 and PARP molecules, which are known to induce apoptosis in the target cells (not shown). The observed apoptosis was abrogated by the pan-caspase inhibitor, z-vad-fmk (10 μM) in D1-1, confirming the requirement of caspases in the apoptosis induced by immobilized epratuzumab (not shown).

Deregulation Of the Cell Cycle—

Immobilized epratuzumab was observed to arrest D1-1 cells in G1 phase of the cell cycle (not shown), while soluble epratuzumab had no effect. Epratuzumab conjugated to beads resulted in about 10% more cells in the G1 phase. A similar increase in the levels of cells was observed in samples treated with anti-IgM or epratuzumab combined with suboptimal amounts of anti-IgM. This deregulation of the cell cycle was associated with changes in the levels of CDK inhibitors, such as p21, p27, and p57 and expression levels of cyclin D1, Rb and phosphorylation of Rb (not shown).

Calcium Release Assay—

We did not observe any release of calcium by immobilized epratuzumab. Also, we did not find an inhibitory effect of epratuzumab or immobilized epratuzumab on the anti-IgM-mediated release of calcium, even after preincubating the cells for 18 h (not shown).

Discussion

In the present study, we confirmed that ligation of mIgM by a sufficient amount of anti-IgM (10 μg/mL) induces the phosphorylation of CD22, CD79a and CD79b, and the localization of all three phosphorylated proteins in the lipid rafts, leading to cell death in the Burkitt D1-I line. We further show that ligation of CD22 with immobilized epratuzumab induces a similar change in CD22, CD79a and CD79b, including phosphorylation, translocation into lipid rafts, and subsequent cell death. Thus, it appears that for a CD22-binding agent to kill Daudi cells in particular, and perhaps other CD22-expressing B-cell lymphomas, two critical events must occur in concert, (i) phosphorylation of CD22, CD79a and CD79b above a threshold level, and (ii) their movement to lipid rafts. This notion is supported by the finding that little or no cell death was observed for D1-1 with either soluble epratuzumab at 50 nM plus a secondary crosslinking antibody or with a suboptimal amount of anti-IgM (1 μg/mL). The former treatment efficiently induced phosphorylation of CD22 and the localization of phospho-CD22 into lipid rafts, but was unable to translocate the weakly phosphorylated CD79a and CD79b to lipid rafts, whereas the latter treatment failed to phosphorylate CD22, CD79a and CD79b at all. On the other hand, combining these two treatments could effect both phosphorylation of CD22, CD79a and CD79b, along with their localization into lipid rafts, and consequently, cell death, as observed for anti-IgM at 10 μg/mL or immobilized epratuzumab.

Binding of CD22 to beads coated with B3 antibody for human CD22 was reported to lower the threshold concentration of anti-IgM required for stimulating DNA synthesis in tonsillar B cells by two orders of magnitude, presumably due to sequestration of CD22 from mIgM by restricting the lateral movement of CD22 in the plane of the cell membrane (Doody et al., 1995, Science 269:242-44). Our immunofluorescence results obtained with D1-1 cells, however, show otherwise, as demonstrated by the colocalization of mIgM and CD22 into a cap-like structure with both soluble epratuzumab and anti-IgM added, and an even more massive coaggregation with epratuzumab immobilized on beads. Thus, we believe that the ability of immobilized epratuzumab to promote such a high degree of mIgM crosslinking without the need for anti-IgM constitutes a sufficient condition for cell killing and negates the inhibitory effect of phosphorylated CD22 in close proximity.

Knowing that binding of CD22 by soluble epratuzumab leads to prompt internalization, and engagement of CD22 with epratuzumab immobilized on plastics should not, raises the question whether internalization of CD22 plays a role in the mechanism of cell killing. Also, the intracellular fate of CD22 after internalization needs to be addressed with experiments designed to determine the kinetics of CD22 recycling, which may reveal that internalized CD22 is predominantly degraded, rather than recycled.

Taking a cue from CD20, which also interacts with BCR and affects calcium mobilization (Walshe et al., 2008, J Biol Chem 283:16971-84) and its own degradation Kheirallah et al., 2010, Blood 115:985-94), the expression levels of CD22 as well as BCR on the cell surface may be critical for the activity of anti-CD22 mAbs, in particular for a non-blocking anti-CD22 mAb like epratuzumab.

Intriguingly, we neither observed any transient increase in intracellular calcium by immobilized epratuzumab nor any inhibitory effect of immobilized epratuzumab on calcium release after stimulation with anti-IgM (not shown). Experiments with longer incubation (16 h) of immobilized epratuzumab followed by stimulation with anti-IgM also did not have any effect on resulting calcium release (not shown). These results were corroborated by a recent finding that a multivalent sialylated polymer synthesized to bind only CD22, but not mIgM, failed to induce any calcium flux (Courtney et al., 2009, Proc Natl Acad Sci USA 106:2500-5), and highlight a key dissimilarity between the mechanism of anti-IgM and immobilized epratuzumab is calcium mobilization, which may require direct engagement of mIgM with anti-IgM. However, resemblances of anti-IgM and immobilized epratuzumab in their characteristic mechanism of action abound, as demonstrated by a similar profile of signal alterations in ERKs, JNKs and p38 MAPK, caspase-dependent apoptosis, change in mitochondria membrane potential, and the generation of ROS.

In conclusion, we provide evidence for the mechanism of action by which immobilized epratuzumab induces cytotoxic and cytostatic effects in CD22-expressing B lymphoma lines (D1-1 and Ramos), both of which have BCR of the IgM isotype. These findings indicate, for the first time, that immobilized epratuzumab and anti-IgM behave similarly in perturbing the BCR-mediated signals in malignant B cells.

### Example 18

### Anti-CD22/CD20 Bispecific Antibody With Enhanced Trogocytosis for Treatment of Lupus

The humanized anti-CD22 mAb, epratuzumab, has demonstrated therapeutic activity in lymphoma and autoimmune diseases. Since epratuzumab only partially depletes circulating B cells, we proposed that its therapeutic activity may result from the modulation of B-cell surface molecules involved in regulating signaling, activation, homing, and re-circulation. Epratuzumab mediates the Fc/FcR-dependent membrane transfer from B cells to effector cells via trogocytosis, resulting in a substantial reduction of multiple B-cell antigen receptor modulators and cell adhesion molecules on the surface of B cells from normal donors or lupus patients. This is the first study of trogocytosis mediated by bispecific antibodies targeting neighboring cell-surface proteins. We show that, compared to epratuzumab, a bispecific hexavalent antibody comprising epratuzumab and veltuzumab (humanized anti-CD20 mAb) exhibits enhanced trogocytosis resulting in major reductions in B-cell surface levels of CD19, CD20, CD21, CD22, CD79b, CD44, CD62L and 137-integrin, and with considerably less immunocompromising B-cell depletion than would result with anti-CD20 mAbs such as veltuzumab or rituximab, given either alone or in combination with epratuzumab. The bispecific antibody is of use for treatment of B-cell diseases, such as B-cell leukemia or lymphoma, immune dysfunction diseases, lupus and other autoimmune diseases, offering advantages over administration of the two parental antibodies in combination.

Introduction

Although the previous view of B cells in autoimmunity was as precursors of deleterious autoantibody-producing plasma cells, they have more recently been ascribed other roles in the pathogenesis of autoimmune diseases, including SLE, such as cytokine production, presentation of autoantigens, promotion of breakdown of T-cell tolerance, and possibly activation of populations of T cells with low affinity toward autoantigens. Due to the central role of B cells in the pathogenesis of autoimmunity, targeted anti-B-cell immunotherapies should offer therapeutic opportunities in the treatment of SLE. Of note, belimumab, which was approved recently for the treatment of SLE, is a mAb that inhibits activation of B cells by blocking B-cell activating factor.

CD22, a B-lymphocyte-restricted member of the immunoglobulin superfamily that regulates B-cell activation and interaction with T cells, is yet another attractive target. The humanized mAb, epratuzumab (hLL2 or IMMU-103), has demonstrated therapeutic activity in clinical trials of lymphoma and autoimmune disease, having treated over 1500 cases of non-Hodgkin lymphoma (NHL), acute lymphoblastic leukemias, Sjögren's syndrome, and SLE. Although epratuzumab has indicated clinical activity, its mechanism of action (MOA) remains obscure. Because epratuzumab has modest antibody-dependent cellular cytotoxicity (ADCC) and negligible complement-dependent cytotoxicity (CDC) in vitro, we postulated that, unlike CD20-targeting mAbs, such as rituximab, its therapeutic action may not result from its moderate depletion of circulating B cells.

Recently, we identified trogocytosis as a previously unknown, and potentially important, MOA of epratuzumab, which may be pertinent to its therapeutic effects in B-cell-regulated autoimmune disease. Trogocytosis, also referred to as shaving, is a mechanism of intercellular communication where two different types of cells initially form an immunological synapse due to the interaction of receptors and ligands on acceptor and donor cells, respectively, after which the ligands and portions of the associated donor cell membrane are taken up and subsequently internalized by the acceptor cell. Importantly, trogocytosis may regulate immune responsiveness to disease-associated antigens and can either stimulate or suppress the immune response. In studies with an ex-vivo model, we demonstrated that epratuzumab mediated a significant reduction of the B-cell surface levels of key B-cell antigen receptor (BCR) signal-modulating proteins, including CD22, CD19, CD21 and CD79b, and also important cell-adhesion molecules, such as CD44, CD62L and β7-integrin, that are involved in B-cell homeostasis, activation, recirculation, migration, and homing. The reduction of the surface proteins on B cells occurred via trogocytosis to FcγR-bearing effector cells, including monocytes, granulocytes and NK cells. Importantly, we verified that these key proteins were reduced significantly on B cells of SLE patients receiving epratuzumab therapy, compared to treatment-naïve patients. We proposed that epratuzumab-mediated loss of BCR modulators and cell-adhesion molecules incapacitates B cells, rendering them unresponsive to activation by T-cell-dependent antigens, leading to therapeutic control in B-cell-mediated autoimmune disease.

The primary MOA of anti-CD20 mAbs in NHL and autoimmune disease is B-cell depletion. Whereas elimination of healthy B cells is likely unavoidable for effective therapy of NHL, it may be detrimental in the therapy of autoimmune diseases due to the increased susceptibility to serious, possibly life-threatening, infections. Although rituximab was approved in 2006 for rheumatoid arthritis, it failed to achieve the primary endpoint in the LUNAR trial of SLE, despite encouraging prior results. Moreover, an analysis of efficacy and safety data from BELONG, a phase III trial of ocrelizumab (humanized anti-CD20), found that the treatment did not significantly improve renal response rates compared with treatment controls, and was associated with a higher rate of serious infections. In both trials, the anti-CD20 mAbs achieved numerically, but not statistically, better responses than the control group, which received standard lupus therapies including steroids, in part because many patients were unable to complete the designed regimen due to serious infections resulting from B-cell depletion. In fact, BELONG was terminated early because of this.

Since both CD20 and CD22 targets have shown activity with their respective antibodies given to patients with autoimmune disease, we postulated that a bispecific antibody (bsAb) targeting both antigens could have superior properties to either parental mAb alone or even a combination of both. Herein, we describe for the first time enhanced trogocytosis mediated by bispecific antibodies targeting neighboring cell-surface proteins. We have developed an anti-CD22/CD20 bispecific hexavalent antibody (bsHexAb), 22*-(20)-(20), that combines the advantages of both anti-CD20 and anti-CD22 therapies, with enhanced trogocytosis and reduced B-cell depletion, compared to the parental anti-CD22 and anti-CD20 mAbs, respectively. This bsAb, which was shown previously to have favorable pharmacokinetics and in vivo stability, could be highly effective in the therapy of autoimmune diseases, including SLE.

Methods

Antibodies, Cell Lines and Reagents.

Epratuzumab (humanized anti-CD22 IgG1κ), veltuzumab (humanized anti-CD20 IgG1κ), labetuzumab (humanized anti-CEACAM5 IgG1κ), and hA19 (humanized anti-CD19 IgG1κ) were provided by Immunomedics, Inc. Rituximab was obtained from a commercial source. The Fc fragment was removed from rituximab and 22*-(20)-(20) by digestion with pepsin at pH 4.0. Daudi and Raji human Burkitt lymphoma cell lines were from ATCC (Manassas, Va.). All cell lines, PBMCs and isolated blood cells were maintained in RPMI 1640 media (Life Technologies, Inc., Gaithersburg, Md.), supplemented with 10% heat inactivated fetal bovine serum (Hyclone, Logan, Utah).

Construction of bsHexAbs.

The construction of 22*-(20)-(20) as a DOCK-AND-LOCK™ (DNL™) complex, and its biochemical characterization are described in the following Example. The 22*-(19)-(19) was assembled using the same method. Independent stable transfectant SpESFX-10 myeloma cell lines produced Cₖ-AD2-IgG-epratuzumab and dimeric C_{H}3-DDD2-Fab modules of veltuzumab and hA19, which were isolated from culture broths by affinity chromatography using MAb-Select and Ni-SEPHAROSE® (GE Healthcare) resins. Cₖ-AD2-IgG-epratuzumab was combined with 2.1 mole equivalents (10% excess) of C_{H}3-DDD2-Fab-veltuzumab or C_{H}3-DDD2-Fab-hA19 to generate 22*-(20)-(20) or 22*-(19)-(19), respectively. DNL conjugations were accomplished by overnight room temperature incubation of the mixtures with 1 mM reduced glutathione, followed by the addition of 2 mM oxidized glutathione. Homogeneous preparations of the bsHexAbs were purified from the reaction mixture with MAb-Select affinity chromatography (data not shown).

Preparation of Blood Cell Fractions.

Heparinized whole blood (buffy coat) from healthy donors was purchased from The Blood Center of New Jersey (East Orange, N.J.). PBMCs were isolated by density gradient centrifugation on UNI-SEP® tubes (Novamed Ltd., Jerusalem, Israel). Depletion of NK cells and isolation of monocytes from PBMCs was accomplished using MACS separation technology (Miltenyi Biotec, Auburn, Calif.) with human anti-CD56 and anti-CD14 microbeads, respectively, according to the manufacturer's recommended protocol.

Ex Vivo Experiments.

Unless indicated differently, PBMCs (1.5×10⁶ cells/mL) were treated in triplicate with 10 μg/mL mAbs or bsHexAbs overnight (16-18 h) at 37° C. in non-tissue culture treated 48-well plates, before analysis by flow cytometry. For each antigen evaluated, incubation with the isotype control labetuzumab (anti-CEACAM5 irrelevant mAb) resulted in fluorescence staining that was indistinguishable from untreated cells. Surface antigen levels, shown as % of control, were obtained by dividing the mean fluorescent intensity (MFI) of the epratuzumab-treated cells by that of the cells treated under the same conditions with labetuzumab, and multiplying the quotient by 100. For B-cell depletion, anti-CD19-PE, anti-CD79b-APC, 7-AAD, and 30,000 COUNTBRIGHT® Absolute Counting Beads (Life Technologies) were added to each tube. For each sample, 8,000 COUNTBRIGHT® beads were counted as a normalized reference. Student's t-test was used to evaluate statistical significance (P<0.05).

Flow Cytometry.

Cell mixtures were stained in a one-step procedure by incubating with mixed fluorochrome-antibody cocktails in 1% BSA-PBS for 30 min at 4° C. Following staining, cells were washed twice with 1% BSA-PBS and samples were acquired on a FACSCALIBUR® flow cytometer (Becton Dickinson, Franklin Lakes, N.J.). For multi-color acquisition, compensation adjustments were performed using single color samples. The same instrument settings were maintained in acquiring all samples. Data were analyzed with Flowjo software (version 7.6.5, Treestar Inc., Ashland, Oreg.). Lymphocytes were gated by forward and side scattering. B cells were identified from the lymphocyte gate using two B-cell specific markers (CD19, CD20, CD22 or CD79b), depending on the specific antibody used for treatment, in order to avoid missing any cells where treatment reduced one marker to near background levels.

Fluorochrome-Antibody Conjugates Used with Flow Cytometry.

The following fluorochrome-anti-human mAbs were used according to the manufacturer's recommendations. Anti-CD22 (FITC and APC, clone HIB22), anti-CD21 (FITC, clone LT21), anti-CD79b (APC and PE, clone CD3-1), and anti-CD19 (PE/Cy7, clone HIB19) were from Biolegend (San Diego, Calif.). Anti-CD19 (PE and FITC, clone LT19) and anti-CD20 (PE, clone LT20), were from Miltenyi Biotec. Anti-CD44 (FITC, clone L178), anti-137 integrin (PE, clone FIB504), and anti-CD62L (FITC, clone DREG-56) were from BD Biosciences (San Jose, Calif.). Binding specificity was confirmed using isotype control mAbs. For exclusion of dead cells, 7-AAD (Life Technologies) was added prior to flow cytometry analysis. Preincubation of PBMCs or Daudi cells with epratuzumab or 22*-(20)-(20) at 4° C. did not inhibit detection of CD22, CD19, CD21, or CD79b with anti-CD22 clone HIB22, anti-CD19 clone HIB19, anti-CD21 clone LT21, or anti-CD79b clone CD3-1, respectively. Preincubation with rituximab, veltuzumab, or 22*-(20)-(20) blocked detection of CD20 anti-CD20 clone LT20. Preincubation with hA19 (humanized anti-CD19) or 22*-(19)-(19) blocked detection of CD19 with anti-CD19 clone LT19 (as well as 11 additional anti-CD19 mAbs).

Fluorescence Microscopy.

Monocytes were purified from freshly isolated PBMCs by positive selection and their plasma membranes were labeled with the PKH26-Red fluorescent cell labeling kit (Sigma, St. Louis, Mo.), following the manufacturer's recommended procedure. Daudi cell plasma membranes were labeled with the PKH67-Green fluorescent cell labeling kit (Sigma). Fluorescent-labeled monocytes and Daudi cells were mixed 2:1 (7.5×10⁶/mL total cell density) and incubated at room temperature for 30 minutes in the presence of 10 μg/mL 22*-(20)-(20) or labetuzumab.

Results

Trogocytosis.

The 22*-(20)-(20) bsHexAb exhibited the broadest and most extensive trogocytosis, reducing each of CD22, CD20, CD19, CD21, CD79b, CD44, CD62L, and β7-integrin more than epratuzumab, and to a similar extent as veltuzumab, except for CD22, which was reduced much more with the 22*-(20)-(20) (Table 9).

TABLE 9
Percent reduction of B-cells antigens following overnight treatment of PBMCs

| Treatmeat | CD22 | CD20 | CD19 | CD21 | CD79b | CD62L | CD44 | β7-Int |
|---|---|---|---|---|---|---|---|---|
| 22*-(20)-(20) | 98 | * | 85 | 78 | 56 | 91 | 52 | 83 |
| 22*-(19)-(19) | 94 | 26 | * | 70 | 46 | 81 | 35 | 56 |
| Epratuzumab | 96 | 16 | 56 | 55 | 42 | 83 | 32 | 64 |
| Veltuzumab | 50 | * | 91 | 84 | 50 | 89 | 54 | 85 |
| hA19 | 27 | 10 | * | 66 | 35 | 66 | 28 | 46 |

Average % reduction from three experiments using PBMCs form independent donors.
* not measured due to blocked detection by the specific treatment.

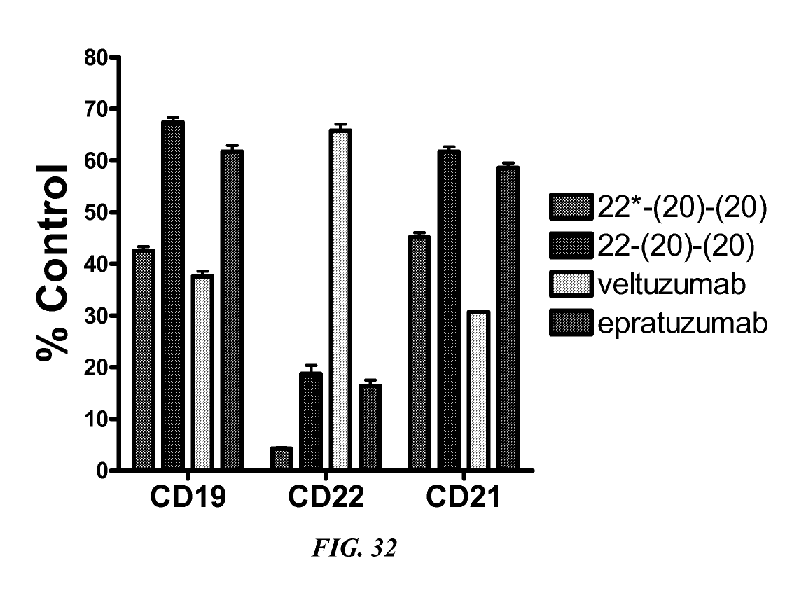

In general, 22*-(19)-(19) showed intermediate trogocytosis, with less antigen reduction than 22*-(20)-(20), but more than epratuzumab for select antigens, such as CD21 and presumably CD19. We were unable to measure CD19 levels following treatment of PBMCs with hA19 or 22*-(19)-(19), because these antibodies block detection of the antigen (12 commercial CD19 mAbs tested). However, the considerable reduction of CD21 suggests a similar reduction of CD19. Similarly, CD20 detection was blocked with veltuzumab or 22*-(20)-(20), although these presumably remove most of the CD20 from B cells. The 22*-(20)-(20) mediated significantly (P<0.001) more trogocytosis compared to 22-(20)-(20), which is a bsHexAb where the additional veltuzumab Fabs are fused at the end of the heavy chain, instead of at the end of the light chain (FIG. 32).

The flow cytometry results demonstrating trogocytosis were further supported by fluorescence microscopy studies (not shown). Purified monocytes and Daudi cells were membrane-labeled with red and green fluorochromes, respectively, and combined. Similar to what was shown with epratuzumab alone, addition of 22*-(20)-(20) to the cell mixture resulted in the rapid formation of immunological synapses and cell clustering between Daudi cells and monocytes, and subsequent trogocytosis of green Daudi membrane components to the red-stained monocytes (not shown). Addition of the control mAb did not result in any evident trogocytosis, even where Daudi cells and monocytes were juxtaposed (not shown).

B-Cell Depletion.

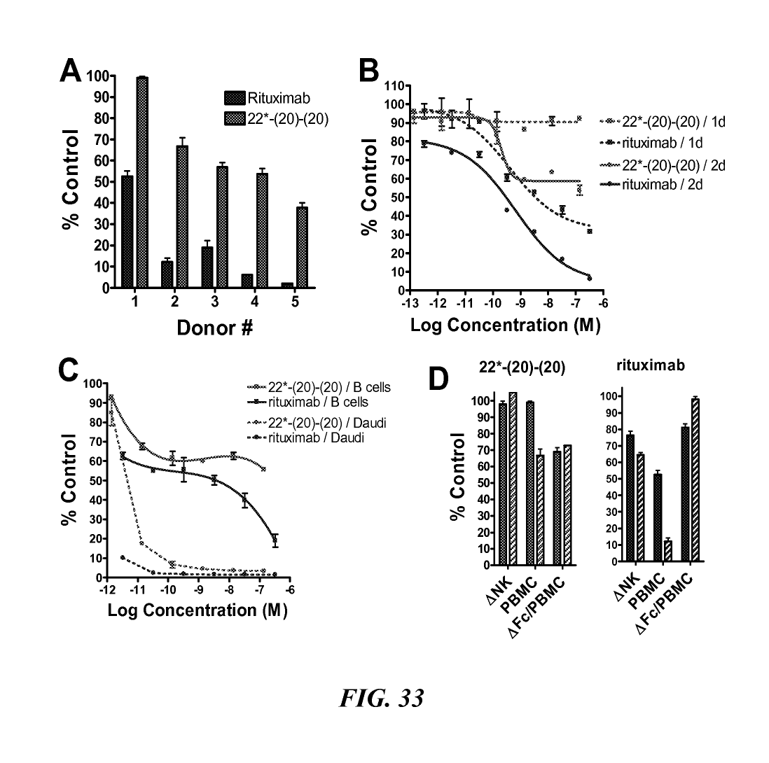

Treatment of PBMCs under the standard experimental conditions used for trogocytosis (10 μg/mL overnight) with either epratuzumab, hA19, or 22*-(19)-(19) caused minimal (<10%) B-cell depletion (data not shown). The B-cell depletion caused by 22*-(20)-(20), specifically as compared to rituximab, was examined with PBMCs from multiple donors, which were treated at various concentrations for two days before counting viable B cells. The maximal level of B-cell depletion varied widely among donors, and for each donor, 22*-(20)-(20) (0-60% depletion) killed significantly (P<0.0001) fewer B cells compared to rituximab (50-98% depletion) (FIG. 33A). As shown using one of the more potent PBMCs (Donor 4), rituximab acted rapidly with considerable depletion after 24 h, whereas 22*-(20)-(20) did not induce appreciable depletion at this time point; however, at higher concentrations of the bsHexAb (>1 nM), significant killing (40%) was evident after 2 days (FIG. 33B). Both 22*-(20)-(20) and rituximab were considerably more effective at killing Daudi cells, which were spiked into PBMCs, compared to normal B cells (FIG. 33C). It is unlikely that CDC is involved, because complement is expected to be removed during PBMC isolation. ADCC, mediated by Fc interactions with NK cells present in the PBMCs, is more likely involved in B-cell depletion. The effect of removal of NK cells (95%) from the PBMCs or deletion of the Fc from the antibodies was examined using weak (Donor 1) and strong (Donor 2) B-cell-depleting PBMCs (FIG. 33D). For rituximab, much less B-cell depletion occurred when NK cells were removed from the PBMCs. It is possible that some ADCC still occurred with residual NK cells or neutrophils that were not eliminated during NK-cell removal and PBMC isolation, respectively. Removal of the Fc from rituximab had an even greater inhibitory effect on B-cell depletion, which was particularly evident with the strong Donor 2. For 22*-(20)-(20), removal of NK cells completely inhibited B-cell depletion with the strong donor. B cells were not depleted from the weak donor, even with intact PBMCs. Unexpectedly, deletion of the Fc from 22*-(20)-(20) did not affect B-cell depletion with the strong donor PBMCs, and markedly increased depletion with the weak donor PBMCs. These results suggest that there are two MOAs of 22*-(20)-(20) engaged in the ex vivo assay. ADCC is inhibited by depletion of NK cells. A putative signaling MOA is inhibited by trogocytosis. Removal of the Fc minimizes ADCC and also inhibits trogocytosis, whereas removal of NK cells only reduces ADCC, and not trogocytosis (not shown), which is mostly mediated by monocytes.

Effector Functions.

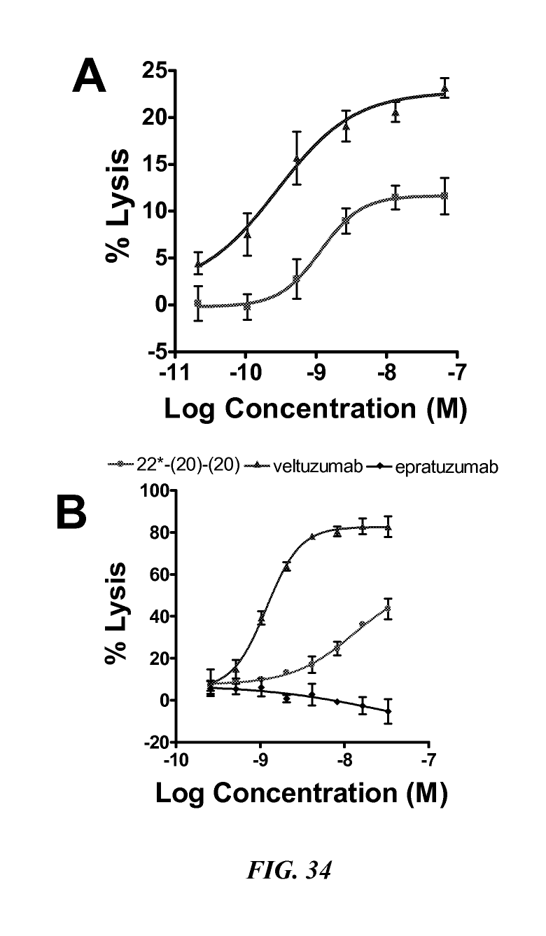

Veltuzumab and rituximab have potent ADCC, whereas hA19 and epratuzumab have moderate and low activity, respectively (data not shown). In repeated experiments using different target cell lines and PBMC donors, the bsHexAb 22*-(19)-(19) exhibited significantly lower ADCC than the humanized anti-CD19 mAb, hA19, and the activity was either similar or marginally higher than epratuzumab, depending on the experiment. The ADCC of 22*-(20)-(20) was compared to that of rituximab with titration experiments. Although the level of ADCC varied among donors, rituximab consistently mediated more killing of Daudi cells, with approximately 2-fold greater maximal lysis compared to 22*-(20)-(20) (FIG. 34A). Neither epratuzumab, hA19, nor 22*-(19)-(19) mediated CDC in vitro (data not shown). The CDC of 22*-(20)-(20) was more than 25-fold less potent than veltuzumab (FIG. 34B).

Discussion

B-cell directed mAbs offer promising therapeutic options for B-cell associated diseases, such as SLE as well as other autoimmune diseases. Epratuzumab has shown clinical efficacy with minimal side-effects in SLE, and is in two worldwide Phase III EMBODY™ registration trials (NCT01262365). Rituximab, and possibly other anti-CD20 mAbs, are associated with increased risks of serious infections, due to near wholesale depletion of B cells. Clinically, epratuzumab depletes only about 35-45% of circulating B cells and does not increase the risk of infection. Nonetheless, epratuzumab is effective in SLE and other diseases by mechanisms that remain unclear.

As disclosed above, we have identified trogocytosis, whereby multiple key proteins, including BCR modulators and adhesion molecules, are stripped from the surface of B cells, as a potentially important MOA of epratuzumab in B-cell regulated autoimmune diseases. We observed that the anti-CD20 mAbs, rituximab and veltuzumab, mediated an even stronger trogocytosis of each antigen (besides CD22). However, the potential of enhanced trogocytosis with anti-CD20 mAbs is diminished, because ultimately the B cells are all killed. Herein, we have identified a novel bsHexAb, 22*-(20)-(20), that mediates a broad and potent trogocytosis of multiple B-cell surface proteins with only moderate B-cell depletion.

An earlier version of an anti-CD22×anti-CD20 bsHexAb, 22-(20)-(20), which has four Fabs of veltuzumab fused to the Fc of epratuzumab, demonstrated potent killing of lymphoma cell lines in vitro. Subsequently, we reported that bsHexAbs of the “Ck” format, with the additional Fabs fused to the end of the light chain, has superior in vivo properties, including pharmacokinetics, neonatal FcR binding, and stability, compared to the original format, where Fabs are fused to the end of the heavy chain. Here, we show that the Ck-based 22*-(20)-(20) mediates more trogocytosis compared to the Fc-based 22*-(20)-(20). This is likely due to a stronger binding affinity for FcγR5 (CD16 and CD64), as was found for FcRn binding.

Trogocytosis with 22*-(20)-(20) reduced the surface levels of CD19, CD21, CD79b, CD44, CD62L, and β7-integrin to similarly low levels as veltuzumab, which were considerably lower than with epratuzumab. Although we were unable to measure the level of CD20 after treatment, it is reasonable to assume that it is reduced to minimal levels, because it is one of the antigens specifically targeted by 22*-(20)-(20) and veltuzumab. Not surprisingly, CD22 is reduced to minimal levels by 22*-(20)-(20), but not with veltuzumab. Trogocytosis, the proposed MOA of epratuzumab, is enhanced with 22*-(20)-(20) by the addition of CD20-binding Fabs to epratuzumab. It is likely that targeting CD20 results in more trogocytosis compared to CD22 targeting, because the former is expressed at a higher level. Another important aspect is that following antibody ligation, CD22, but not CD20, is rapidly internalized, which is expected to compete with trogocytosis.

Previously, we reported that the Fc-based bsHexAb, 22-(20)-(20), does not internalize rapidly, and it is likely that this is also the case for 22*-(20)-(20). The broad and potent trogocytosis mediated by 22*-(20)-(20) may modulate immune B cells more effectively than epratuzumab.

The key advantage of trogocytosis with 22*-(20)-(20) over rituximab or veltuzumab is that the bsHexAb kills less B cells. The extent of B-cell depletion varied considerably using PBMCs from different donors. “Weak” PBMCs had almost no B-cell depletion with 22*-(20)-(20) (50% with rituximab), whereas with “strong” PBMCs, up to 60% of the B cells were depleted with the bsHexAb and nearly 100% were killed with rituximab. Presumably, ADCC is the chief MOA involved in B-cell depletion in the ex vivo assay. We have found that in-vitro ADCC is highly variable among donors, which likely is responsible for the variability in B-cell depletion. We have observed a correlation between ADCC potency and B-cell depletion with a small number of PBMC specimens that were tested for both activities; however, a systematic study was not performed. Closer inspection of the dose-response curves suggests a biphasic shape, indicating that more than one MOA might be involved in the B-cell killing in the ex vivo assays (FIG. 33B and FIG. 33C). Removal of NK cells from the PBMCs, which is expected to eliminate ADCC, completely inhibited B-cell depletion with 22*-(20)-(20). Conversely, removal of the Fc, which eliminates trogocytosis as well as ADCC, resulted in enhanced B-cell depletion. This suggests that the second MOA is a result of the direct action on B cells, and is inhibited by trogocytosis. Previously, we described in-vitro cytotoxicity with the Fc-based 22-(20)-(20) on NHL cell lines resulting from signaling mechanisms involving Lyn, Syk, PLCγ2, AKT and NF-κB pathways leading to apoptosis via signaling transduction mechanisms. The Fc-based bsHexAb also caused some ex-vivo depletion of B cells even though it has weak ADCC, suggesting that normal B-cell death resulted from signaling. The current results indicate that 22*-(20)-(20) also can induce apoptosis of normal B cells. However, stripping the antigens from the cell surface by trogocytosis diminishes the effects of signaling. This does not appear to be the case with rituximab, because removal of its Fc eliminates B-cell depletion. Although CDC is eliminated from the ex vivo system, it is likely to play a role in vivo. That 22*-(20)-(20) has considerably lower CDC than rituximab could widen the difference in B-cell depletion resulting from immunotherapy with these antibodies.

In this study, we compared two bsHexAbs, each comprising epratuzumab fused at the end of its light chains with four additional Fab fragments to either CD20 or CD19. In general, 22*-(20)-(20) induced more trogocytosis than 22*-(19)-(19), which reduced many of the proteins to a similar extent as epratuzumab. However, CD21, and presumably CD19, were reduced more with 22*-(19)-(19), compared to epratuzumab. Although we believe that 22*-(20)-(20) is a more promising therapeutic candidate for SLE, 22*-(19)-(19), having enhanced trogocytosis of some antigens and minimal B-cell depletion, may also be therapeutically useful.

The potentially ideal effects that might result from immunotherapy with 22*-(20)-(20), specifically, the extensive reduction via trogocytosis of many key B-cell surface proteins, including CD20, CD22, CD19 and CD21, with only moderate B-cell depletion, cannot be accomplished with a mixture of the two parent mAbs. While a mixture of veltuzumab (or rituximab) and epratuzumab may result in a similarly broad trogocytosis as the bsHexAb, inclusion of the anti-CD20 mAb will cause massive depletion of circulating B cells, rendering SLE patients susceptible to serious infections. Further, infusion of two mAbs, instead of a single agent, would be less convenient for both physicians and patients. Thus, 22*-(20)-(20) offers an improved next-generation antibody for the therapy of SLE and other autoimmune diseases, without the risk associated with rituximab or other potent anti-CD20 mAbs.

### Example 19

### Production and Use of DNL™ Complexes Showing Improved Stability, Pharmacokinetics and Efficacy by Attaching AD Moieties to the C-Terminal End of the Antibody Light Chain

We explored the production and use of improved Dock-and-Lock™ (DNL™) complexes, incorporating IgG molecules with an AD moiety fused to the C-terminal end of the kappa light chain (hereafter denoted as “Cₖ” complexes or fusion proteins), instead of the C-terminal end of the Fc (hereafter denoted as “C_{H}”). In the Examples below, the Cₖ DNL™ complexes are also indicated by an asterisk (e.g., 20*-2b). Two exemplary Cₖ-derived prototypes, an anti-CD22/CD20 bispecific hexavalent antibody, comprising epratuzumab (anti-CD22) and four Fabs of veltuzumab (anti-CD20), and a CD20-targeting immunocytokine, comprising veltuzumab and four molecules of interferon-α2b, displayed enhanced Fc-effector functions in vitro, as well as improved pharmacokinetics, stability and anti-lymphoma activity in vivo, compared to their Fc-derived counterparts. These unexpected superior results favor the use of DNL™ conjugates with the Cₖ-design for clinical development.

The Cₖ-IgG-IFNα, designated 20*-2b, had a similar molecular size and composition to its Fc-IgG-IFNα counterpart, 20-2b, each comprising veltuzumab and 4 copies of IFNα2b fused at the C-terminal ends of the light or heavy chains, respectively. The Cₖ-bsHexAb, designated 22*-(20)-(20), and its Fc-bsHexAb homologue, 22-(20)-(20), each comprised epratuzumab and 4 veltuzumab Fabs, which were fused at the C-terminal ends of the light and heavy chains, respectively. Compared to the analogous Fc-based immunoconjugates, the Cₖ-IgG-IFNα and Cₖ-bsHexAb were more stable in vivo, cleared more slowly from the circulation and had improved Fc-effector function, significantly enhancing efficacy in vivo.

Methods

Antibodies And Cell Culture—

Immunomedics provided veltuzumab (anti-CD20 IgG1), epratuzumab (anti-CD22 IgG₁), a murine anti-IFNα mAb, hMN-14 (labetuzumab), a rat anti-idiotype mAb veltuzumab (WR2), and a rat anti-idiotype mAb to epratuzumab (WN). HRP-conjugated second antibodies were from Jackson Immunoresearch (Westgrove, Pa.). Heat-inactivated fetal bovine serum (FBS) was obtained from Hyclone (Logan, Utah). All other cell culture media and supplements were purchased from Invitrogen Life Technologies (Carlsbad, Calif.). SpESFX-10 cells (Rossi et al., 2011, Biotechnol. Prog. 27:766-775) and production clones were maintained in H—SFM. Daudi cell line was purchased from ATCC and grown in 10% FBS-RPMI (Manassas, Va.).

DNL™ Constructs—

Methods for production of Cₖ-based DNL™ constructs are described in further detail below. For C_{H}3-AD2-IgG-veltuzumab, C_{H}3-AD2-IgG-epratuzumab, C_{H}1-DDD2-Fab-veltuzumab, and IFNα2b-DDD2, generation of the mammalian expression vectors and production clones, and their use for the DNL™ conjugation of 20-2b and 22-(20)-(20), have been reported previously (Rossi et al., 2008, Cancer Res. 68:8384-8392; Chang et al., 2009, Bioconjug. Chem. 20:1899-1907; Rossi et al., 2009, Blood 114:3864-3871; Rossi et al., 2009, Blood 113:6161-6171). Cₖ-AD2-IgG, was generated by recombinant engineering, whereby the AD2 peptide was fused to the C-terminal end of the kappa light chain. Because the natural C-terminus of C_{K} is a cysteine residue, which forms a disulfide bridge to C_{H}1, a 16-amino acid residue “hinge” linker (SEQ ID NO:122) was used to space the AD2 from the C_{K}-V_{H}1 disulfide bridge. The goal of this approach was to obtain full binding and activities of all Fabs and effector groups, while maintaining a full Fc effector function. The ultimate goal was to maintain a Pk that approaches that of IgG and prevent the intracellular dissociation of the modules, which presumably occurs by proteolysis following uptake of the complex into the cell.

The first C_{K}-AD2-IgG module was constructed for veltuzumab (hA20), with additional C_{K}-AD2-IgG modules produced subsequently for milatuzumab (hLL1), epratuzumab (hLL2) and hR1 (anti-IGF-1R). These modules have been used to generate hexavalent antibodies and immunocytokines, which were compared to constructs of similar composition that were made with the corresponding C_{H}3-AD2-IgG modules. The mammalian expression vectors for Cₖ-AD2-IgG-veltuzumab and Cₖ-AD2-IgG-epratuzumab were constructed using the pdHL2 vector, which was used previously for expression of the homologous C_{H}3-AD2-IgG modules. A 2208-bp nucleotide sequence (SEQ ID NO:130) was synthesized comprising the pdHL2 vector sequence ranging from the Bam HI restriction site within the V_{K}/C_{K} intron to the Xho I restriction site 3′ of the Cₖ intron, with the insertion of the coding sequence for the hinge linker (EFPKPSTPPGSSGGAP, SEQ ID NO:122) and AD2 in frame at the 3′ end of the coding sequence for C_{K}. This synthetic sequence was inserted into the IgG-pdHL2 expression vectors for veltuzumab and epratuzumab via Bam HI and Xho I restriction sites. Generation of production clones with SpESFX-10 were performed as described for the C_{H}3-AD2-IgG modules (Rossi et al., 2008, Cancer Res. 68:8384-8392; Rossi et al., 2009, Blood 113:6161-6171). Cₖ-AD2-IgG-veltuzumab and Cₖ-AD2-IgG-epratuzumab were produced by stably-transfected production clones in batch roller bottle culture, and purified from the supernatant fluid in a single step using MABSELECT™ (GE Healthcare) Protein A affinity chromatography.

Following the same process described previously for 22-(20)-(20) (Rossi et al., 2009, Blood 113:6161-6171), Cₖ-AD2-IgG-epratuzumab was conjugated with C_{H}1-DDD2-Fab-veltuzumab, a Fab-based module derived from veltuzumab, to generate the bsHexAb 22*-(20)-(20), where the 22* indicates the Cₖ-AD2 module of epratuzumab and each (20) symbolizes a stabilized dimer of veltuzumab Fab. The properties of 22*-(20)-(20) were compared with those of 22-(20)-(20), the homologous Fc-bsHexAb comprising C_{H}3-AD2-IgG-epratuzumab (not shown), which has similar composition and molecular size, but a different architecture.

Following the same process described previously for 20-2b (Rossi et al., 2009, Blood 114:3864-3871), Cₖ-AD2-IgG-veltuzumab, was conjugated with IFNα2b-DDD2, a module of IFNα2b with a DDD2 peptide fused at its C-terminal end, to generate 20*-2b, which comprises veltuzumab with a dimeric IFNα2b fused to each light chain. The properties of 20*-2b were compared with those of 20-2b (not shown), which is the homologous Fc-IgG-IFNα. Each of the bsHexAbs and IgG-IFNα were isolated from the reaction mixture by MABSELECT™ affinity chromatography.

Production of DNA Vectors for the Expression of C_{K}-AD2-IgG Modules.—

A 2208 basepair DNA sequence (SEQ ID NO:130) was synthesized, comprising the sequence of the pdHL2 expression vector from the Bam HI restriction site (within the V_{K}/C_{K} intron) to the Xho I restriction site (preceding the heavy chain expression cassette), with the insertion of the coding sequence for the hinge linker (SEQ ID NO:122) and AD2 (SEQ ID NO:4), in frame at the 3′ end of the coding sequence for C_{K}. This synthetic sequence was inserted into the Bam HI/XhoI restriction sites in the expression vector for veltuzumab (hA20-pdHL2) in a single cloning step, to generate C_{K}-AD2-IgG-hA20-pdHL2 (not shown). Similarly, the 2208 basepair fragment was inserted into the pGSHL expression vectors for epratuzumab, milatuzumab and hR1 using Bam HI/Xho I restriction sites (not shown).

The synthetic nucleic acid sequence for conversion of IgG-pdHL2 to CK-AD2-IgG-pdHL2 vector is shown in SEQ ID NO:130. 5′ Bam HI and 3′ Xho I restriction sites are underlined. The coding sequence for the C_{K}-hinge linker-AD2 peptide is shown in bold.

(SEQ ID NO: 130)
GGATCCCGCAATTCTAAACTCTGAGGGGGTCGGATGACGTGGCCATTCTT
TGCCTAAAGCATTGAGTTTACTGCAAGGTCAGAAAAGCATGCAAAGCCCT
CAGAATGGCTGCAAAGAGCTCCAACAAAACAATTTAGAACTTTATTAAGG
AATAGGGGGAAGCTAGGAAGAAACTCAAAACATCAAGATTTTAAATACGC
TTCTTGGTCTCCTTGCTATAATTATCTGGGATAAGCATGCTGTTTTCTGT
CTGTCCCTAACATGCCCTGTGATTATCCGCAAACAACACACCCAAGGGCA
GAACTTTGTTACTTAAACACCATCCTGTTTGCTTCTTTCCTCAGGAACTG
TGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCC
AGGAGAGTGTCACAGAGCAGGACAGCAAGGACAGCACCTACAGCCTCAGC
AGCACCCTGACGCTGAGCAAAGCAGACTACGAGAAACACAAAGTCTACGC
CTGCGAAGTCACCCATCAGGGCCTGAGCTCGCCCGTCACAAAGAGCTTCA
ACAGGGGAGAGTGTGAGTTCCCTAAACCCAGCACTCCACCCGGATCTTCC
GGCGGCGCTCCCTGTGGCCAGATCGAGTACCTGGCCAAGCAGATCGTGGA
CAACGCCATCCAGCAGGCCGGGTGCTAGAGGGAGAAGTGCCCCCACCTGC
TCCTCAGTTCCAGCCTGACCCCCTCCCATCCTTTGGCCTCTGACCCTTTT
TCCACAGGGGACCTACCCCTATTGCGGTCCTCCAGCTCATCTTTCACCTC
ACCCCCCTCCTCCTCCTTGGCTTTAATTATGCTAATGTTGGAGGAGAATG
AATAAATAAAGTGAATCTTTGCACCTGTGGTTTCTCTCTTTCCTCATTTA
ATAATTATTATCTGTTGTTTTACCAACTACTCAATTTCTCTTATAAGGGA
CTAAATATGTAGTCATCCTAAGGCGCATAACCATTTATAAAAATCATCCT
TCATTCTATTTTACCCTATCATCCTCTGCAAGACAGTCCTCCCTCAAACC
CACAAGCCTTCTGTCCTCACAGTCCCCTGGGCCATGGTAGGAGAGACTTG
CTTCCTTGTTTTCCCCTCCTCAGCAAGCCCTCATAGTCCTTTTTAAGGGT
GACAGGTCTTACAGTCATATATCCTTTGATTCAATTCCCTGAGAATCAAC
CAAAGCAAATTTTTCAAAAGAAGAAACCTGCTATAAAGAGAATCATTCAT
TGCAACATGATATAAAATAACAACACAATAAAAGCAATTAAATAAACAAA
CAATAGGGAAATGTTTAAGTTCATCATGGTACTTAGACTTAATGGAATGT
CATGCCTTATTTACATTTTTAAACAGGTACTGAGGGACTCCTGTCTGCCA
AGGGCCGTATTGAGTACTTTCCACAACCTAATTTAATCCACACTATACTG
TGAGATTAAAAACATTCATTAAAATGTTGCAAAGGTTCTATAAAGCTGAG
AGACAAATATATTCTATAACTCAGCAATTCCCACTTCTAGGGGTTCGACT
GGCAGGAAGCAGGTCATGTGGCAAGGCTATTTGGGGAAGGGAAAATAAAA
CCACTAGGTAAACTTGTAGCTGTGGTTTGAAGAAGTGGTTTTGAAACACT
CTGTCCAGCCCCACCAAACCGAAAGTCCAGGCTGAGCAAAACACCACCTG
GGTAATTTGCATTTCTAAAATAAGTTGAGGATTCAGCCGAAACTGGAGAG
GTCCTCTTTTAACTTATTGAGTTCAACCTTTTAATTTTAGCTTGAGTAGT
TCTAGTTTCCCCAAACTTAAGTTTATCGACTTCTAAAATGTATTTAGAAT
TTCGACCAATTCTCATGTTTGACAGCTTATCATCGCTGCACTCCGCCCGA
AAAGTGCGCTCGGCTCTGCCAAGGACGCGGGGCGCGTGACTATGCGTGGG
CTGGAGCAACCGCCTGCTGGGTGCAAACCCTTTGCGCCCGGACTCGTCCA
ACGACTATAAAGAGGGCAGGCTGTCCTCTAAGCGTCACCACGACTTCAAC
GTCCTGAGTACCTTCTCCTCACTTACTCCGTAGCTCCAGCTTCACCAGAT
CCCTCGAG

Production and Purification of C_{K}-AD2-IgG Modules—

The C_{K}-AD2-IgG-hA20-pdHL2 vector was linearized by digestion with Sal I restriction enzyme and transfected into SpESFX-10 myeloma cells by electroporation. Following electroporation, the cells were plated in 96-well tissue culture plates and transfectant clones were selected with 0.05 μM methotrexate (MTX). Clones were screened for protein expression by sandwich ELISA using wells coated with WR2 (hA20 anti-Id) and detection with peroxidase-conjugated goat anti-human Fab.

The three C_{K}-AD2-IgG-pGSHL expression vectors were transfected similarly to above, but plated in glutamine-free media for selection, instead of MTX. Clones were screened for protein expression by sandwich ELISA using wells coated with antibody-specific anti-Ids and detection with peroxidase-conjugated goat anti-human Fab.

The highest producing clones were expanded and cultured in roller bottles for protein expression. The C_{K}-AD2-IgG modules were purified using Protein A affinity chromatography. The productivity of the cell lines was similar to that of IgG or C_{H}3-AD2-IgG. Reducing SDS-PAGE resolved a protein band for the hA20 Kappa-AD2 polypeptide with a relative mobility consistent with its calculated molecular weight (26,951 Da) and larger than hA20 Kappa (23,204 Da) (not shown). Expectedly, the heavy chain polypeptides of Cₖ-AD2-IgG-hA20 co-migrated with those of hA20 IgG.

Bispecific Hexavalent Antibodies Made by DNL™ with C_{K}-AD2-IgG—

Bispecific hexavalent antibodies (bsHexAbs) were generated by combining Cₖ-AD2-IgG modules with C_{H}3-DDD2-Fab modules of a different specificity and performing DNL™ conjugation under mild redox conditions. Six bsHexAbs and one monospecific HexAb were produced and characterized, as exemplified by the construct named 20Cₖ-(74)-(74) (alternatively, 20*-(74)-(74)), where the first code (20Cₖ or 20*) indicates the Ck-AD2-IgG module and codes in parentheses indicate stabilized dimeric Fab-DDD2 modules. Thus, 20Cₖ-(74)-(74) (or 20*-(74)-(74)) comprises veltuzumab (anti-CD20) fused with four anti-CD74 Fabs derived from milatuzumab. The component parts and valencies of the 7 HexAbs are given in Table 10.

TABLE 10
Bispecific hexavalent antibodies targeting B-cell malignancies.

| | | | Valency | | |
|---|---|---|---|---|---|
| HexAb | IgG-AD2 module (parent mAb) | Fab-DDD2 module (parent mAb) | CD20 | CD22 | CD74 |
| 20Ck-(22)-(22) | Cₖ-AD2-IgG-hA20 (veltuzumab) | C_{H}3-DDD2-Fab-hLL2 (epratuzumab) | 2 | 4 | |
| 20Ck (74)-(74) | Cₖ-AD2-IgG-hA20 (veltuzumab) | C_{H}3-DDD2-Fab-hLL1 (milatuzumab) | 2 | | 4 |
| 20Ck-(20)-(20) | Cₖ-AD2-IgG-hA20 (veltuzumab) | C_{H}3-DDD2-Fab-hA20 (veltuzumab) | 6 | | |
| 22Ck-(20)-(20)* | Cₖ-AD2-IgG-hLL2 (epratuzumab) | C_{H}3-DDD2-Fab-hA20 (veltuzumab) | 4 | 2 | |
| 22Ck-(74)-(74) | Cₖ-AD2-IgG-hLL2 (epratuzumab) | C_{H}3-DDD2-Fab-hLL1 (milatuzumab) | | 2 | 4 |
| 74Ck-(20)-(20) | Cₖ-AD2-IgG-hLL1 (milatuzumab) | C_{H}3-DDD2-Fab-hA20 (veltuzumab) | 4 | | 2 |
| 74Ck-(22)-(22) | Cₖ-AD2-IgG-hLL1 (milatuzumab) | C_{H}3-DDD2-Fab-hLL2 (epratuzumab) | | 4 | 2 |

*monospecific hexavalent

Each of the HexAbs was produced and purified in a similar fashion. A detailed description of one preparation of 22*-(20)-(20) is provided as an example. A molar excess of C_{H}3-DD2-Fab-hA20 (42 mg) was mixed with 25 mg of C_{K}-AD2-IgG-hLL2 in Tris-Citrate buffer (pH 7.5±0.2). Reduced glutathione and EDTA were added at 2 mM and 1 mM, respectively, and the reaction was held overnight at room temperature, prior to addition of 4 mM oxidized glutathione and an additional 4-hour incubation at room temperature. The reaction mixture was applied to a 5-ml MABSELECT™ (Protein A) chromatography column, which was washed with PBS prior to elution of the bsHexAb with 0.1M Citrate, pH 3.5. The 22*-(20)-(20) construct was dialyzed into 0.04M PBS, pH 7.4. A total of 56 mg of 22*-(20)-(20) was recovered, representing 96% yield. Size exclusion HPLC (SE-HPLC) resolved a single homogeneous protein peak with a retention time consistent with a protein of ˜368 kDa molecular weight (not shown). The SE-HPLC peak for the Cₖ-AD2-based bsHexAbs resolve with a slightly longer retention time compared to the corresponding C_{H}3-AD2-based bsHexAbs (not shown), which have a similar composition and molecular weight, indicating that the former have a smaller Stokes radius and are more compact molecules, compared to the latter.

20(Cₖ)-2b, an IgG-IFNα Immunocytokine Based on Cₖ-AD2-IgG-hA20—

An immunocytokine comprising veltuzumab fused with four IFNα2b groups was prepared using the DNL™ method by combining Cₖ-AD2-IgG-hA20 with IFNα2b-DDD2. C_{K}-AD2-IgG-hA20 (54 mg) was combined with 81.1 mg of IFNα-DDD2. EDTA (1 mM) and reduced glutathione (2 mM) were added and the solution was held for 5 hours at room temperature. Oxidized glutathione (4 mM) was added to the mixture, which was held overnight at room temperature. The 20*-2b was purified to near homogeneity using two sequential affinity chromatography steps. First, the reaction mixture was applied to a 4-ml MABSELECT™ (Protein A) column. Protein was eluted with 4 column volumes (16 ml) of 0.02% Polysorbate-80, 50 mM citrate, pH 3.5 directly into 16 ml of 0.02% P-80, 80 mM imidazole, 1 M NaCl, 100 mM Na₂HPO₄ and the solution was adjusted to pH 7.3 with 50 mM Na₂HPO₄, 40 mM imidazole, 500 mM NaCl. The adjusted eluent was applied to an 8-ml Ni-SEPHAROSE® 6 FF column equilibrated with 0.02% P-80, 40 mM imidazole, 0.5 M NaCl, 50 mM NaPO₄, pH 7.5. A total of 85 mg of 20(Cₖ)-2b was eluted with 5 column volumes of 500 mM imidazole, 0.02% P-80, 50 mM NaCl, 20 mM NaH₂PO₄, pH 7.5.

SE-HPLC resolved a major protein peak for 20*-2b with a retention time consistent with a protein of ˜250 kDa (not shown). The 20*-2b peak resolved with a longer retention time than that of 20-2b, which comprises the same components (veltuzumab and four IFNα2b) and has a similar molecular weight, indicating that the former has a smaller Stokes radius and is more compact than the latter, similar to what was observed for the HexAbs.

Analytical Methods—

Size-exclusion high performance liquid chromatography (SE-HPLC) was performed using a 4 μm UHR SEC column (Waters Corp., Milford Mass.). SDS-PAGE was performed using 4-20% gradient Tris-glycine gels (Invitrogen, Gaithersburg, Md.). IEF was performed at 1000 V, 20 mM and 25 watts for 1 h, using pH 6-10.5 ISOGEL® Agarose IEF plates (Lonza, Basel, Switzerland) on a BIO-PHORESIS® horizontal electrophoresis cell (Bio-Rad, Hercules, Calif.). All colorimetric (ELISA and MTS) and fluorometric (CDC and ADCC) assays were quantified with an ENVISION® 2100 Multilabel Plate Reader (PerkinElmer, Waltham, Mass.).

Cell Binding—

Binding to cells was measured by flow cytometry on a GUAVA® PCA using GUAVA® Express software (Millipore Corp., Billerica, Mass.). Veltuzumab and 20*-2b were labeled with phycoerythrin (PE) using a ZENON® R-Phycoerythrin human IgG labeling kit following the manufacturer's protocol (Invitrogen, Molecular Probes). Daudi cells were incubated with the PE-veltuzumab and PE-20*-2b (0.1-15 nM) for 30 min at room temperature and washed with 1% BSA-PBS prior to analysis. Plots of concentration vs. mean fluorescence intensity (MFI) were analyzed by linear regression.

In Vitro Cytotoxicity—

Daudi cells were plated at 10,000 cells/well in 96-well plates and incubated at 37° C. for 3 days in the presence of increasing concentrations of 20*-2b or 20-2b. Viable cell densities were determined using the MTS-based CELLTITER 96® Cell Proliferation Assay (Promega, Madison, Wis.).

FcRn Binding Measurements—

FcRn binding was evaluated by surface plasmon resonance on a BIACORE® X instrument (GE Healthcare) following the methods of Wang et al. (2011, Drug Metab Dispos. 39:1469-1477). Soluble single-chain FcRn was generated following the methods of Feng et al. (2011, Protein Expr. Pur 79:66-71). The extracellular domain of the human FcRn heavy chain was fused with β2-microglobulin via a flexible peptide linker. The fusion protein was expressed using a modified pdHL2 vector in transfectant SpESFX-10 cells, and purified using Ni-Sepharose. Purified scFcRn was immobilized onto a CM5 biosensor chip using an amine coupling kit (GE Healthcare) to a density of ˜600 response units (RU). The test articles were diluted with pH 6.0 running buffer [50 mM NaPO₄, 150 mM NaCl, and 0.05% (v/v) Surfactant 20] to 400, 200, 100, 50, and 25 nM and bound to the immobilized scFcRn for 3 min to reach equilibrium, followed by 2 min of dissociation with the flow rate at 30 μL/min. The sensorchip was regenerated with pH 7.5 running buffer between runs. To determine FcRn binding affinity (K_{D}) at pH 6.0, the data from all five concentrations were used simultaneously to fit a two-state reaction model (BIAevaluation software; GE Healthcare). Goodness of fit was indicated by χ² values.

Pk Analyses—

The pharmacokinetics (Pk) and in vivo stability were compared between 20*-2b and 20-2b following intravenous (i.v.) or subcutaneous (s.c) injection in mice. Groups of 18 Swiss-Webster mice were administered 1-mg doses of 20*-2b or 20-2b by either i.v. or s.c. injection. Using 3 mice per time point, animals were sacrificed and bled at 6, 16, 24, 48, 72 and 96 hours. Therefore, each serum sample represented an independent animal/time point. For measurement of intact and total (intact plus dissociated) IgG-IFNα, microtiter wells were adsorbed with WR2, a rat anti-Id for veltuzumab, at 5 μg/mL in 0.5 M Na₂CO₃, pH 9.5. Following blocking with 2% BSA-PBS, serum dilutions in antibody buffer (0.1% gelatin, 0.05% proclin, 0.05% Tween-20, 0.1 M NaCl, 0.1 M NaPO₄, pH 7.4) were incubated in the coated wells for 2 h. For measurement of intact IgG-IFNα, wells were probed with a mouse anti-IFNα mAb (5 μg/mL in antibody buffer) for 1 h, followed by detection with HRP-conjugated goat anti-mouse IgG-Fc. For measurement of total veltuzumab IgG, wells were probed with HRP-conjugated goat anti-human IgG-Fc for 1 h.

For measurement of intact and total bsHexAbs, microtiter wells were adsorbed with WN, a rat anti-idiotype for epratuzumab. Serum dilutions were incubated in the coated wells for 2 h. For detection of intact bsHexAb, wells were probed with HRP-conjugated WR2 (1 μg/mL in antibody buffer) for 1 h. For detection of total epratuzumab IgG, wells were probed with HRP-conjugated goat anti-human IgG-Fc for 1 h.

Signal was developed with o-phenylenediamine dihydrochloride substrate solution and OD was measured at 490 nM. The concentrations of intact and total species were extrapolated from construct-specific standard curves. Pk was analyzed using the WINNONLIN® Pk software package (v5.1; Pharsight Corp.; Mountain View, Calif.).

In Vivo and Ex Vivo Methods—

Injection and collection of sera from rabbits was performed by Lampire Biological Laboraories (Pipersville, Pa.). For Pk studies, 10-week old male Swiss-Webster mice (Taconic, Germantown, N.Y.) and New Zealand White rabbits were injected subcutaneously (SC), and also intravenously (IV) for mice, with test agents diluted in PBS. Blood samples were obtained by cardiac puncture and from the ear vein for mice and rabbits, respectively. Serum was isolated from clotted blood by centrifugation, and diluted in antibody buffer, prior to analysis by ELISA.

Human blood specimens were collected from healthy donors. In-vitro ADCC and CDC activity were assayed as described previously (Rossi et al., 2008, Cancer Res. 68, 8384-8392). For ADCC, Daudi cells were incubated for 4 h at 37° C. with PBMCs, which were isolated from the blood of healthy donors, at a 50:1 effector:target ratio using test agents at 33 nM.

In Vivo Efficacy in Mice—

Female 8-12-week old C.B.17 homozygous SCID mice (Taconic) were inoculated intravenously with 1.5×10⁷ Daudi cells on day 0. For comparison of the bsHexAbs, treatment was administered by SC injection on days 1 and 5. For comparison of the IgG-IFNα, treatments were administered as a single SC injection on day 7. Saline was used as a control treatment. Animals, monitored daily, were humanely euthanized when hind-limb paralysis developed or if they became otherwise moribund. Additionally, mice were euthanized if they lost more than 20% of initial body weight. Survival curves were analyzed using Kaplan-Meier plots, using the Prism (v4.03) software package (GraphPad Software, Inc., San Diego, Calif.). Some outliers determined by critical Z test were censored from analyses.

Statistical Analyses—

Statistical significance (P<0.05) was determined using student's T-tests for all results except for the in vivo survival curves, which were evaluated by log-rank analysis.

Results

Synthesis of Cₖ-Based Immunoconjugates—

The DNL™ synthesis produced homogeneous preparations of 22*-(20)-(20), 22-(20)-(20), 20*-2b and 20-2b. By SDS-PAGE (non-reducing), each conjugate was resolved into a tight cluster of bands with relative mobility conforming to their expected size (data not shown), and under reducing conditions, only bands representing the constituent polypeptides for each conjugate were evident, demonstrating a high degree of purity (not shown). For each conjugate, SE-HPLC resolved a major peak having a retention time consistent with their molecular size (not shown). The longer retention times observed for 22*-(20)-(20) and 20*-2b are likely due to their more compact structure, as compared to 22-(20)-(20) and 20-2b, respectively. Isoelectric focusing showed that 20*-2b and 20-2b have a similar pI (calculated pI=pH 7.22), with no evidence of unreacted IgG-AD2 (pI=pH 7.86) or IFNα2b-DDD2 (pI=pH 6.87) modules (not shown).

Both conjugates retain full binding of the parental mAbs, as shown for 20*-2b, which exhibited identical binding as veltuzumab to live Daudi cells (not shown). Cytotoxicity also was similar between the Cₖ and Fc versions in Daudi cells (EC₅₀=0.2 μM), demonstrating equivalent CD20 binding and IFNα specific activity (not shown).

Pharmacokinetics—

We reported previously that the T_{1/2} for Fc-bsHexAbs were approximately half as long as their parental mAbs in mice (Rossi et al., 2009, Blood 113:6161-6171). In the initial study, which measured the serum concentrations of 22*-(20)-(20), 22-(20)-(20) and epratuzumab in mice over a period of 72 h after subcutaneous (SC) injection (not shown), 22-(20)-(20) reached maximal concentration at 16 h and was cleared with a T_{1/2} about 1 day, similar to the findings before. In comparison, both epratuzumab and 22*-(20)-(20) reached peak levels between 24 and 48 h, while clearing similarly, but slower than 22-(20)-(20). A subsequent study monitoring clearance over 5 days again found 22*-(20)-(20) with superior Pk, showing ˜2-fold higher maximum concentration in serum, with longer T_{1/2} and mean residence time (MRT), culminated in a 3.8-fold greater area under the curve (AUC). (Table 11).

As in mice, the Pk parameters determined in rabbits were ˜2-fold greater for 22*-(20)-(20), resulting in a 3.3-fold greater AUC, compared to 22-(20)-(20) (Table 12). Importantly, the concentrations of the 22*-(20)-(20) following SC administration in both mice and rabbits were sustained for longer periods.

TABLE 11
Summary of pharmacokinetic parameters

| | | Dose | | T_{1/2} | Tₘₐₓ | Cₘₐₓ | AUC_{(0-∞)} | MRT |
|---|---|---|---|---|---|---|---|---|
| Species | Route | (mg) | Construct | (h) | (h) | (μg/mL) | (h*μg/ml) | (h) |
| Mouse | IV | 1.0 | 20*-2b | 36.2 | 6.0 | 649.0 | 32516.5 | 55.2 |
| | | | 20-2b | 17.1 | 6.0 | 629.8 | 15514.0 | 19.1 |
| Mouse | SC | 1.0 | 20*-2b | 37.9 | 16.0 | 312.1 | 18318.2 | 62.1 |
| | | | 20-2b | 16.0 | 16.0 | 146.0 | 6498.6 | 30.9 |
| Mouse | SC | 0.5 | 22*-(20)-(20) | 106.5 | 24.0 | 50.6 | 6704.7 | 153.1 |
| | | | 22-(20)-(20) | 54.5 | 16.0 | 26.5 | 1752.9 | 85.2 |
| Mouse | SC | 18 | 22*-(20)-(20) | 117.9 | 53.3 | 31.6 | 6079.1 | 179.6 |
| | | | 22-(20)-(20) | 51.1 | 37.3 | 17.8 | 1838.4 | 89.2 |

T_{1/2}, elimination half-life;
Tₘₐₓ, time of maximal concentration;
Cₘₐₓ, maximal concentration;
AUC, area under the curve;
MRT, mean residence time.

Binding affinity (K_{D}) of the bsHexAbs to the neonatal Fc receptor (FcRn) was assessed by surface plasmon resonance and found to be 166 and 310 nM for 22*-(20)-(20) and 22-(20)-(20), respectively (P=0.01). The affinity of epratuzumab (16 nM) was approximately 10-fold stronger than 22*-(20)-(20) (P=0.007) (Table 11).

TABLE 12
Summary of Biacore analysis for neonatal Fc receptor binding affinity

| | Epratuzumab | | | 22*-(20)-(20) | | | 22-(20)-(20) | | |
|---|---|---|---|---|---|---|---|---|---|
| | k_{d} | kₐ | K_{D} | k_{d} | kₐ | K_{D} | k_{d} | kₐ | K_{D} |
| Run | 0.0242 | 1.64 × 10⁶ | 15.0 | 0.0395 | 2.80 × 10⁵ | 141.1 | 0.0458 | 1.48 × 10⁵ | 309.5 |
| 1 | | | | | | | | | |
| Run | 0.0218 | 1.56 × 10⁶ | 17.9 | 0.0404 | 2.26 × 10⁵ | 178.8 | 0.0411 | 1.54 × 10⁵ | 266.9 |
| 2 | | | | | | | | | |
| Run | 0.0239 | 1.56 × 10⁶ | 15.8 | 0.0441 | 2.48 × 10⁵ | 177.8 | 0.0419 | 1.19 × 10⁵ | 352.1 |
| 3 | | | | | | | | | |
| Mean ± | 0.0233 ± | 1.59 × 10⁶ ± | 16.3 ± | 0.0413 ± | 2.51 × 10⁵± | 165.9 ± | 0.0429 ± | 1.40 × 10⁵ ± | 309.5 ± |
| S.D | 0.0013 | 4.62 × 10⁴ | 1.5 | 0.0024 | 2.72 × 10⁴ | 21.5 | 0.0025 | 1.87 × 10⁴ | 42.6 |

k_{d} = 1/s;
kₐ = 1/Ms;
K_{D} = k_{d}/kₐ given as nM concentration

Fc-IgG-IFNα constructs, such as 20-2b, also were cleared from circulation faster than their parental mAb (Rossi et al., 2009, Blood 114:3864-3871). However, when the Pk parameters of 20*-2b and 20-2b following either SC or intravenous (IV) injection were compared (not shown), the T_{1/2}, Cₘₐₓ, and MRT were each again about 2-fold higher for 20*-2b, resulting in a 2.8-fold greater AUC, compared to 20-2b (Table 12). For IV administration, 20*-2b had a 2- and 2.8-fold longer T_{1/2} and MRT, respectively, and a 2-fold greater AUC.

In Vivo Stability—

The Fc-bsHexAbs and Fc-IgG-IFNα are stable ex vivo in serum (Rossi et al., 2009, Blood 114:3864-3871; Rossi et al., 2009, Blood 113:6161-6171). However, analysis of serum samples from earlier Pk studies suggested these constructs dissociate in vivo over time, presumably by intracellular processing. We compared the in vivo stability of 20*-2b and 20-2b by measuring the concentrations of the intact IgG-IFNα and the total veltuzumab, which allowed for differentiating the intact from the dissociated species (not shown). The % intact IgG-IFNα was plotted versus time (not shown), and in vivo dissociation rates for 20-2b and 20*-2b were calculated by linear regression to 0.97%/h and 0.18%/h, respectively. A similar analysis was performed on serum samples following SC injection of the bsHexAbs in mice, with in vivo dissociation rates for 22-(20)-(20) and 22*-(20)-(20) calculated to 0.55%/h and 0.19%/h, respectively (not shown). Interestingly, both 22-(20)-(20) and 22*-(20)-(20) were completely stable in vivo following SC injections in rabbits (not shown). The reason for the difference in in vivo stabilities between mice and rabbits is not known.

Effector Function—

We reported that Fc-IgG-IFNα and Fc-bsHexAbs did not induce measurable CDC in vitro, even when their parental mAb had potent activity (Rossi et al., 2009, Blood 114:3864-3871; Rossi et al., 2009, Blood 113:6161-6171). Consistent with the prior results, veltuzumab exhibited strong CDC, yet no activity was evident for 20-2b (not shown). Hoever, 20*-2b induced strong CDC, which approached the potency of veltuzumab (not shown a). Under these in vitro conditions, epratuzumab lacked CDC, whereas 22-(20)-(20) achieved a modest increase, and 22*-(20)-(20) induced even greater activity, which was ˜10-fold less potent than veltuzumab (not shown).

Unlike CDC, the Fc-based conjugates did not have reduced ADCC, but instead, 20-2b exhibited enhanced ADCC compared to veltuzumab (Rossi et al., 2009, Blood 114:3864-3871). Depending on the PBMC donor, epratuzumab induced little or no ADCC in vitro, and not surprisingly, 22-(20)-(20) did not show a statistically significant improvement (not shown). However, the ADCC associated with 22*-(20)-(20) was not significantly different from veltuzumab, when PBMCs of a high-ADCC donor were used (not shown). With a low-ADCC PBMC donor, 22*-(20)-(20) had enhanced activity (11.4% lysis), compared to epratuzumab (2.3%) and 22-(20)-(20) (4.3%), but it was lower than veltuzumab (18.5%) (P=0.0326, data not shown).

In vivo Efficacy—

As reported previously, 20-2b is remarkably potent in treating mice bearing human Daudi Burkitt lymphoma xenografts, which are highly sensitive to direct killing by IFNα (Rossi et al., 2009, Blood 114:3864-3871). Using the same model, the Cₖ-based conjugates demonstrated even more potent anti-tumor activity than their Fc-based counterparts (not shown). While both 20-2b and 20*-2b at a single 1 μg-dose cured the majority of the animals, with median survival time (MST) greater than 189 days, 20*2b, but not 20-2b, at 0.25 μg maintained its potency, providing evidence of significantly improved therapeutic efficacy (MST >189 days with 7/8 cures for 20*2b vs. 134.5 days with just 3/8 survivors for 20-2b; P=0.0351). A molar equivalent of veltuzumab (0.6 μg) to 1 μg of 20-2b increased the MST by only 12.5 days over saline control, The superiority of another different Cₖ construct over the Fc-parental construct was shown again in the disseminated Daudi model, where animals were administered two injections (days 1 and 5) of high (1 mg) or low (10 μg) doses of 22*-(20)-(20) or 22-(20)-(20) (not shown). For the high dose, the MST was >123 and 71 days with 100% and 10% survival for 22*-(20)-(20) and 22-(20-(20), respectively (P<0.0001). With the low-dose treatment, the MST was 91 days for 22*-(20)-(20) with 2 mice surviving, compared to 50.5 days for 22-(20-(20) with no survivors (P=0.0014). High doses of each bsHexAb improved survival significantly more (P<0.0001) than either epratuzumab alone or in combination with C_{H}1-DDD2-Fab-veltuzumab, which were given at a molar equivalent to the 1-mg dose of bsHexAb. At the 100-fold lower dosing, both bsHexAbs were superior to high-dose epratuzumab (P<0.003), and 22*-(20)-(20), but not 22-(20)-(20), was superior to high-dose epratuzumab plus C_{H}1-DDD2-Fab-veltuzumab (P<0.0001).

Discussion

The various formats of antibody-based fusion proteins, including bsAbs (Kontermann, 2010, Curr Opin Mol Ther 12:176-183) and immunocytokines (Kontermann, 2012, Arch Biochem Biophys 526:194-205), can largely be categorized into three groups, based on where additional moieties are fused to a whole IgG, an Fc, or an antigen-binding fragment such as Fab, scFv or diabody. Whereas Fc-fusion may increase T_{1/2}, and fusion to antigen-binding fragments should impart targeting, only fusion to IgG could expect to achieve antibody targeting, full Fc effector function and markedly extended Pk. Because not all IgG-fusion designs are created equal, effector activities and Pk are known to vary widely among the different formats and even between particular constructs of the same design.

DNL™ complexes are exceptional for producing immunoconjugates that retain full antigen-binding avidity of the targeting antibody and biological activity of the appending effector molecules (e.g., cytokines), and have potent efficacy both in vitro and in vivo (Rossi et al., 2012, Bioconjug. Chem. 23:309-323; Rossi et al., 2009, Blood 114:3864-3871; Rossi et al., 2009, Blood 113:6161-6171; Rossi et al., 2010, Cancer Res. 70:7600-7609; Rossi et al., 2011, Blood 118:1877-1884). However, Fc-bsHexAbs and Fc-IgG-IFNα were cleared from circulation at approximately twice the rate of their parental mAbs. Sub-optimal Pk is a common deficiency associated with immunoconjugates that is primarily attributed to impaired dynamic binding to the FcRn (Kuo & Aveson, 2011, MAbs. 3:422-430). To improve Pk, we engineered a new class of IgG-AD2 module having the AD2 peptide fused at the C-terminal end of the light chain. The new module was used to assemble Cₖ-bsHexAbs and Cₖ-IgG-IFNα, which not only exhibited comparable in vitro properties to their Fc-based homologues, including antigen binding, IFNα specific activity and in vitro cytotoxicity, but also had superior Pk, in vivo stability and Fc effector activity, which together resulted in increased in vivo efficacy, compared to the already potent Fc-based counterparts.

The superior Pk of the Cₖ-bsHexAbs and Cₖ-IgG-IFNα is most likely attributed to their increased binding affinity to the FcRn, which was twice as strong at pH 6.0 for 22*-(20)-(20), compared to 22-(20)-(20). FcRn binding is mediated by portions of the C_{H}2 and C_{H}3 domains of IgG, with critical contact sites located near the C-terminal end of the Fc (Huber et al., 1993, J Mol Biol 230:1077-1083; Raghavan et al., 1994, Immunity. 1:303-315). Considering that the T_{1/2} of 22*-(20)-(20) was in the range of epratuzumab (Rossi et al., 2009, Blood 113:6161-6171), it was unanticipated that FcRn binding was approximately 10-fold weaker for the former (155 nM). However, using this same method, we measured the FcRn K_{D} at 42 and 92 nM for other humanized mAbs, which typically have Pk similar to epratuzumab (data not shown). T_{1/2} is not necessarily directly correlated with FcRn K_{D} at pH 6.0 (Dall'Acqua, 2002, J Immunol 169:5171-5180; Gurbaxani et al., 2006, Mol Immunol 43, 1462-1473). It has been suggested that the rate of dissociation at pH 7.4 is equally or perhaps more important in determining T_{1/2} (Wang, 2011, Drug Metab Dispos. 39:1469-1477). Although FcRn:IgG contacts are limited to the Fc domain, the antigen-binding domain can negatively impact FcRn binding, as evidenced by the fact that most therapeutic antibodies share a very similar Fc (IgG₁), yet vary widely in FcRn K_{D} and T_{1/2} (Suzuki, 2010, J Immunol 184:1968-1976). Additional factors include endocytosis, ligand:antibody ratio, antibody structural stability, antibody pI, and methionine oxidation (Kuo & Aveson, 2011, MAbs. 3:422-430).

For fusion proteins, the FcRn K_{D} and T_{1/2} can be influenced by the nature and location of the fusion partner (Suzuki et al., 2010, J Immunol 184:1968-1976; Lee et al., 2003, Clin Pharmacol Ther 73, 348-365). We observed that the T_{1/2} of each IgG-IFNα was shorter than the corresponding bsHexAb that was assembled using the same class of IgG-AD2 module. For example, the T_{1/2} of 20*-2b (37.9 h) was markedly shorter than that of 22*-(20)-(20) (106.5 h), suggesting that, independent of their location, the IFNα groups negatively impact FcRn binding, perhaps by lowering the pI of the adduct.

The present Example identifies the C-terminal end of the light chain as the most advantageous location for fusion to IgG. An immunocytokine of single-chain IL-12 fused to the N-terminal end of the heavy chain of an anti-HER2 IgG₃ retained HER2 binding (Peng et al., 1999, J Immunol 163:250-258)¹⁷. We applied a similar strategy using DNL™ by constructing an IgG-AD2 module having the AD2 peptide fused to the N-terminal end of veltuzumab heavy chain. However, bsHexAbs and IgG-IFNα made with this module did not bind CD20 on cells (data not shown). This might have been because of the large size of the additional (Fab)₂ or (IFNα2b)₂ groups. That these conjugates bound to anti-idiotype mAbs suggests that the nature of the antigen, which is a small extracellular loop of CD20, might be a factor. The C-terminal end of the heavy chain is the most common and convenient location for fusion to IgG (Kontermann, 2012, Arch Biochem Biophys 526:194-205). However, this is also the most likely location to impact FcRn binding and Pk negatively. For example, an immunocytokine of GM-CSF fused at the C-terminus of the heavy chain of an anti-HER2 IgG₃ exhibited markedly reduced T_{1/2} (10 hours) compared to the parental mAb (110 hours) (Dela Cruz et al., 2000, J Immunol 165:5112-5121). Fc-based bsAbs also suffer from diminished Pk. As an example, a bsAb having an anti-IGF-1R scFv fused to the C-terminal end of the heavy chain of an anti-EGFR IgG cleared from circulation in mice twice as fast (T_{1/2}=9.93 h), compared to the parental mAb (T_{1/2}=20.36 h) (Dong et al., 2011, MAbs. 3:273-288).

Croasdale and colleagues systematically studied the effect of fusion location with IgG-scFv tetravalent bsAbs using an anti-IGF-1R IgG₁ fused at the N- or C-termini of the heavy or light chains, with an anti-EGFR scFv (Croasdale et al., 2012, Arch. Biochem Biophys 526:206-18). Fusion of scFv to the IgG at the C-terminus of the light chain produced the highest yields, had the longest T_{1/2} and was the most effective in vivo. The authors indicated that each construct bound FcRn and FcγRIIIa; however, K_{D} was not reported. Among the different formats, the C-terminal heavy chain fusion had the shortest T_{1/2}. Fusion at the N- or C-terminus of the heavy chain resulted in substantially reduced or complete loss, respectively, of ADCC. Alternatively, fusion at the C-terminus of the light chain did not decrease ADCC.

Our results show that fusion location can impact ADCC. For the bsHexAbs comprising epratuzumab as the IgG, which has minimal ADCC, strong ADCC was measured for 22*-(20)-(20), but not 22-(20)-(20), suggesting that this Fc effector function was provided by the addition of the four anti-CD20 Fabs, and that their fusion location is critical. Additionally, 22*-(20)-(20) showed moderate CDC, which was not detected for epratuzumab, and only modestly increased for 22-(20)-(20), suggesting that this effector function also can be bestowed to a CDC-lacking mAb by the addition of Fabs of a CDC-inducing mAb, and that, the activity is sensitive to the location of the fusion site. This was demonstrated clearly with the IgG-IFNα, where the Fc-IgG-IFN, 20-2b, did not have detectable CDC and 20*-2b induced potent activity, similar to veltuzumab.

Although the Fc-bsHexAbs and Fc-IgG-IFNα are quite stable in human or mouse sera and whole blood (Rossi et al., 2009, Blood 114:3864-3871; Rossi et al., 2009, Blood 113:6161-6171), the Fc-fusions, in particular, were not completely stable in vivo. The Fc-based conjugates dissociated at a rate of 0.5-1.0%/h in mice, compared to <0.2%/h for the Cₖ-based constructs. Because dissociation has never been observed ex vivo, we presume it occurs by an intracellular process. Interestingly, there was no evidence of in-vivo instability in rabbits, even after 5 days. The C-terminal lysine residue of the heavy chain is often cleaved proteolytically during antibody production. The common Fc-based fusion proteins, where additional groups are fused to the C-terminal lysine, potentially can be cleaved in vivo by proteases, such as plasmin, which cleave after exposed lysine residues (Gillies et al., 1992, Proc Natl Acad Sci U.S. A 89, 1428-1432).

In summary, this study demonstrates the superior in vivo properties of bsAbs and immunocytokines made as DNL™ complexes with fusion at the C-terminal end of the light chain, suggesting that the C-terminus of the light chain is the preferred fusion location for most immunoconjugates with intended clinical use.

### Example 20

### Production and Use of Cₖ-Based DNL™ Complexes for Treatment of Autoimmune Disease

Background

Systemic lupus erythematosus (SLE) has been classified as an autoimmune disease that may involve many organ systems, as an inflammatory multisystem rheumatic disorder, or as a collagen vascular disease. Corticosteroids remain the foundation for long-term management with most patients, even those in clinical remission, maintained using low doses. High-dose steroids, particularly 0.5-1.0 g pulse i.v. methylprednisolone, are standard treatment for management of an acute flare, with immunosuppressants (azathioprine, cyclophosphamide, methotrexate, etc.) generally used in severe cases when other treatments are ineffective. The cytotoxicity associated with immunosuppressants as well as the problems of long-term systemic corticosteroid therapy provide incentives to develop targeted and less toxic therapies, particularly those with steroid-sparing effects. No new agent has been approved as a therapeutic for SLE in over 50 years until the recent approval of Benlysta (belimumab) in March of 2011.

Although the conventional view of B cells is as precursors of immunoglobulin-producing plasma cells, they may also play other roles in the pathogenesis of SLE, such as presenting autoantigens, promoting the breakdown of peripheral T-cell tolerance, and possibly by activating populations of T cells with low affinity toward autoantigens (Looney, 2010, Drugs 70:529-540; Mok, 2010, Int J Rheum Dis 13:3-11; Goldenberg, 2006, Expert Rev Anticancer Ther 6:1341-1353; Thaunat et al., 2010, Blood 116:515-521). Because of the central role of B cells in the pathogenesis of autoimmunity, targeted anti-B-cell immunotherapies are expected to offer therapeutic value in for SLE. For example, Benlysta is a monoclonal antibody (mAb) that inhibits activation of B cells by blocking B-cell activating factor.

Another B-cell target is CD22, a 135-kD glycoprotein that is a B-lymphocyte-restricted member of the immunoglobulin superfamily, and a member of the sialoadhesin family of adhesion molecules that regulate B cell activation and interaction with T cells (e.g., Carnahan et al., 2007, Mol Immunol 44:1331-1341; Haas et al., 2006, J Immunol 177:3063-3073; Haas et al., 2010, J Immunol 184:4789-4800). CD22 has 7 extracellular domains and is rapidly internalized when cross-linked with its natural ligand, producing a potent costimulatory signal in primary B cells. CD22 is an attractive molecular target for therapy because of its restricted expression; it is not exposed on embryonic stem or pre-B cells nor is it normally shed from the surface of antigen-bearing cells.

Epratuzumab is a humanized anti-CD22 antibody that is in advanced clinical trials. Clinical trials with epratuzumab have been undertaken for patients with non-Hodgkin's lymphoma, leukemias, Waldenstrom's macroglobulinemia, Sjögren's syndrome and SLE, and encompass an experience in more than 1000 patients. In the initial clinical study with epratuzumab in non-Hodgkin's lymphoma (NHL) or other B-cell malignancies, patients received 4 consecutive weekly epratuzumab infusions at doses of ranging from 120 to 1000 mg/m²/week (Goldenberg, 2006, Expert Rev Anticancer Ther 6:1341-1353; Goldenberg et al., 2006, Arthritis Rheum 54:2344-2345; Leonard et al., 2003, J Clin Oncol 21:3051-3059; Leonard et al., 2008, Cancer 113:2714-2723). Treatment-related toxicity typically involved nausea, chills/rigors, fever and other transient mild or moderate infusion reactions occurring primarily during the first weekly infusion. Peripheral B-cell levels decreased following epratuzumab therapy, but otherwise no consistent changes were seen in RBC, ANC, platelets, immunoglobulins, or T-cell levels following treatment. Epratuzumab blood levels after the 4^{th} weekly infusion increased in a dose-dependent manner, and epratuzumab remained in circulation with a half-life of 23 days. Interestingly, enhanced anti-tumor effects in indolent and aggressive forms of NHL were reported when epratuzumab was combined with the anti-CD20 rituximab (Goldenberg, 2006, Expert Rev Anticancer Ther 6:1341-1353; Leonard et al., 2005, J Clin Oncol 23:5044-5051; Leonard et al., 2008, Cancer 113:2714-2723; Strauss et al., 2006, J Clin Oncol 24:3880-3886). Epratuzumab is entering two Phase-III registration trials for the treatment of SLE patients. Also noteworthy is that rituximab has also shown activity in patients with SLE (Ramos-Casals et al., 2009, Lupus 18:767-776).

Since the combination of rituximab and epratuzumab showed improved anti-lymphoma efficacy without increased toxicity in patients (Leonard et al., 2008, Cancer 113:2714-2723), we engineered and evaluated bsAbs against both CD20 and CD22, including an earlier design based on the IgG-(scFv)₂ format (Qu et al., 2008, Blood 111:2211-2219) and the more recent DNL™ design based on the hexavalent IgG-(Fab)₄ format, which resulted in 22-(20)-(20) and 20-(22)-(22) (Rossi et al., 2009 Blood 113:6161-6171). Specifically, 22-(20)-(20) comprises epratuzumab and 4 additional Fabs of veltuzumab, and thus binds CD22 bivalently and CD20 tetravalently. Likewise, the other bsAb, 20-(22)-(22), comprising veltuzumab and 4 Fabs of epratuzumab, binds CD20 bivalently and CD22 tetravalently. For the original HexAbs, referred to henceforth as C_{H}3-HexAbs, the two types of modules are C_{H}3-AD2-IgG and C_{H}1-DDD2-Fab. Each of these modules is produced as a fusion protein in myeloma cells and purified by protein A (C_{H}3-AD2-IgG) or KappaSelect (C_{H}1-DDD2-Fab) affinity chromatography.

A HexAb can be either monospecific or bispecific. The C_{H}3-HexAbs comprise a pair of Fab-DDD2 dimers linked to a full IgG at the carboxyl termini of the two heavy chains, thus having six Fab-arms and a common Fc domain. For example, the code of 20-(22)-(22) designates the bispecific HexAb comprising a divalent anti-CD20 IgG of veltuzumab and a pair of dimeric anti-CD22 Fab-arms of epratuzumab, whereas 22-(20)-(20) specifies the bispecific HexAb comprising a divalent anti-CD22 IgG of epratuzumab and a pair of dimeric anti-CD20 Fab-arms of veltuzumab.

As discussed in Example 19 above, we have developed an alternative HexAb format by utilizing a new IgG-AD2 module, Cₖ-AD2-IgG, which has the AD2 peptide fused to the carboxyl end of the kappa light chain, instead of at the end of the Fc. Combination with C_{H}1-DDD2-Fab results in a Cₖ-HexAb structure, having a different architecture, but similar composition (6 Fabs and an Fc), to the C_{H}3-HexAb, having the four additional Fabs linked at the end of the light chain. As discussed above, the Cₖ-HexAbs exhibit superior effector functions and have significantly improved pharmacokinetics (Pk), compared to the original C_{H}3-HexAbs.

The Cₖ-HexAbs are particularly well suited for in vivo applications as they display favorable Pk, are stable in vivo, and may be less immunogenic as both DDD- and AD-peptides are derived from human proteins and the constitutive antibody components are humanized. In addition, each of the anti-CD22/CD20 potently induce direct cytotoxicity against various CD20/CD22-expressing lymphoma and leukemic cell lines in vitro without the need for a secondary antibody to effect hypercrosslinking, which is required for the parental mAbs. In vivo studies confirmed the efficacy of the bsAbs to inhibit growth of Burkitt lymphoma xenografts in mice, thus indicating their larger size has not affected tumor targeting and tissue penetration.

Preliminary Results

Clinical Experience with Epratuzumab—

Clinical studies have been conducted to examine the efficacy of epratuzumab in indolent and aggressive forms of NHL alone in combination with rituximab. The published data show that the antibody can be given weekly for 4 weeks in a <1-h infusion up to doses of 1,000 mg/m², with the optimal dose appearing to be 360 mg/m², and resulting in very durable objective responses in 43% of follicular patients given this optimal dose, with one-third comprising CRs. When combined weekly for four weeks with rituximab, follicular, indolent NHL patients showed an overall 67% objective response (7% PR and 60% CR/Cru), with only one patient relapsing at 19 months follow-up. We and others have studied the combination of rituximab and epratuzumab at their recommended full doses weekly×4 in multicenter US and European trials, with results indicating a higher CR rate than observed in historical studies with rituximab alone in similar patient populations.

For lupus, completed studies have enrolled 331 unique individuals who received at least one dose of epratuzumab (Shoenfeld et al. (Eds.), Immunotherapeutic Agents for SLE. Future Medicine Ltd; 2012). In the initial study, Dörner et al. administered 360 mg/m² to 14 patients with moderately active SLE (Dorner et al., 2006, Arthritis Res Ther 8:R74). Patients received 360 mg/m² epratuzumab intravenously every 2 weeks for four doses, with analgesic/antihistamine premedications (but no steroids), and were followed for up to 32 weeks. The drug had effects as early as 6 weeks, with 93% demonstrating improvements in British Isles Lupus Activity Group (BILAG) Index in at least one B- or C-level disease activity at 6 weeks, and all patients achieved improvement in at least one BILAG body system at 10 weeks. Epratuzumab was well tolerated and had a median infusion time of 32 min. Blood B-cell levels decreased by an average of 35% at 6 weeks and remained decreased at 6 months post-therapy. No adverse safety signals were detected. B-cell levels decreased post-treatment by about 40%, which is much less than the experience with anti-CD20 mAbs. Post-treatment T-cell levels, immunoglobulins and other standard safety laboratory tests remained unchanged from baseline. No evidence of HAHA was found in these patients. No consistent changes in autoantibodies and other disease-related laboratory tests were seen.

This led to two Phase III studies known as ALLEVIATE I and II (SL0003/SL0004; ClinicalTrials.gov registry: NCT00111306 and NCT00383214) that were intended to be 48-week, randomized, double-blind, placebo-controlled trials, followed by an open-label, long-term, safety study for patients in the USA (SL0006). The protocol included infusing patients with epratuzumab at 360 or 720 mg/m² (in addition to background therapy, which included corticosteroids and immunosuppressives) over four consecutive 12-week cycles: in the first cycle, four infusions were given at weeks 0, 1, 2 and 3; for the three subsequent retreatment cycles, two infusions were given at weeks 0 and 1. The primary efficacy end point was a BILAG responder analysis at week 12, since too few patients completed the originally intended 24 patient response variable evaluation. Responders had a reduction of BILAG A or B by one level, no new BILAG A or less than two new Bs, and no introduction of immunosuppression or increase in steroid doses during the treatment period. Initiated in 2005, the study was prematurely discontinued in 2006 due to drug supply interruption. At that point, only 90 patients had been studied long enough for analysis and the two groups were pooled.

A total of 29 US patients were given open-label follow-up therapy in SL0006. Subjects generally had serious lupus: the mean BILAG score was 13.2 and 43% had at least one BILAG A. In total, 63% were on immunosuppressive agents and 43% were on 25 mg or more of prednisone daily. A total of 91% received four infusions and 69% reached week 24. Using an intention-to-treat analysis, a BILAG response was achieved at week 12 in 44.1, 20 and 30.3% of the 360 mg/m², 720 mg/m² and placebo groups, respectively, with responses seen within 6 weeks. Epratuzumab demonstrated significant steroid-sparing properties and correlated with improvements in health-related quality of life. The improvements were sustained in those who stayed in the open label follow-up. No significant intergroup differences were found in adverse events or serious adverse events. B-cell depletion was approximately 20-40% among treated patients.

EMBLEM was a Phase IIb, 12-week, double-blind study of six different dosing regimens for patients with at least one BILAG A and/or two BILAG B's (ClinicalTrials.gov registry: NCT00624351). This study included 227 SLE patients with a mean total BILAG score of 15.2 and a mean SLE disease activity index of 14.8 who were on a mean 14 mg of prednisone daily, and the majority were also taking immunosuppressive agents. Study participants thus had more multisystem disease activity than has been seen in any other published lupus clinical trial. Four weekly infusions, two infusions every other week, or placebo, were given against a background of prednisone and, for most, immunosuppressive therapy. Those who received a combined dose of 2400 mg had meaningful and statistically significant improvements, with 37.9% achieving an ‘enhanced BILAG improvement’, whereby at least two levels (e.g., A to C, B to D) of improvement were noted. Again, there were no safety signals or significant immunosuppression. Only four out of 187 (2.1%) patients developed HAHA.

EMBODY, a pivotal 48-week trial consisting of two large cohorts totaling nearly 2000 patients, was initiated in December 2010 (ClinicalTrials.gov registry: NCT01262365).

Clinical Experience with Veltuzumab—

We have studied veltuzumab in over 80 NHL patients refractory/relapsed to prior therapies, including rituximab, and it has been found to have about a 43% objective response and a 27% complete response rate in FL patients at all doses summarized, which appear to be durable (15-25 months) (Morchhauser et al., 2009, J Clin Oncol 27:3346-3353). Activity was seen even at doses of 80 mg/m². Importantly, the infusion profile appears better than rituximab's, with no grade 3-4 adverse reactions and infusion times of <2 h (compared to 4-8 h for rituximab). Veltuzumab has been examined also in a subcutaneous (SC) formulation in B-cell lymphoma (Negrea et al., 2011, Haematologica 96:567-573).

Veltuzumab has also been studied in patients with immune thrombocytopenia (ITP) (ClinicalTrials.gov registry: NCT00547066), and has been shown to be active in this disease, even when low doses have been administered (twice, on weeks 1 and 3) intravenously and subcutaneously (data not shown). Forty-one patients received 2 doses of veltuzumab 2 weeks apart. Veltuzumab was well-tolerated (limited Grade 1-2 transient reactions, except one Grade 3 infusion reaction), with no other safety issues. Of 38 assessable patients, 9 with newly-diagnosed or persistent disease (ITP ≦1 year) previously treated only with steroids and/or immunoglobulins, had 7 (78%) responses including 3 (33%) CRs and 4 (44%) PRs, while 29 with chronic disease up to 31 years and additional prior therapies had 20 (69%) responses, including 4 (13%) CRs and 10 (35%) PRs. For the 27 responders, median time to relapse increased with response category (MR: 2.4, PR: 5.5, CR: 14.4 months) with 10 (37%) responding >1 year (3 ongoing at 3.0-3.8 years). Eight responders were retreated, with 3 again achieving PRs, including one retreated 4 times. Both IV and SC routes depleted B cells after the first injection at all doses. Eight patients developed low HAHA titers of uncertain clinical significance. Thus, veltuzumab is a promising therapeutic on its own, both in NHL and in an autoimmune disease.

Hexavalent bsAbs Made by DNL™—

The molecular engineering, production, purification and biochemical/biological characterization of 22-(20)-(20) and 20-(22)-(22) have been reported (Rossi et al., 2009, Blood 113:6161-6171). A detailed examination of the mechanism of action and cell signaling induced by 22-(20)-(20) and 20-(22)-(22) has also been published (Gupta et al., 2010, Blood 116:3258-3267). The key findings are as follows.

Both 22-(20)-(20) and 20-(22)-(22) retained the binding properties of their parental Fab/IgGs with all 6 Fabs capable of binding simultaneously. Competitive ELISAs showed that each construct possesses the functional valency as designed, and that each Fab binds with a similar affinity to those of the parental mAb. Flow cytometry demonstrate bispecific binding to live NHL cells with longer retention than the parental mAbs. The internalization rate of the bsAbs is largely influenced by both valency and the internalizing nature of the constitutive antibodies. Specifically, 22-(20)-(20) with four Fabs from the slowly internalizing veltuzumab and two Fabs from the rapidly internalizing epratuzumab behaves similar to veltuzumab, showing a slow internalization rate. Conversely 20-(22)-(22) with four Fabs from the rapidly internalizing epratuzumab and two Fabs from the slowly internalizing veltuzumab exhibits rapid internalization, similar to epratuzumab.

The two anti-CD20/CD22 bsAbs induced caspase-independent apoptosis more potently than veltuzumab or epratuzumab, either alone or in combination. Despite the incorporation of veltuzumab, which alone displays potent CDC, neither bsAb is able to induce CDC. Both bsAbs exhibit ADCC, with 20-(22)-(22) more potent than 22-(20)-(20), presumably due to the higher density of CD20 than CD22 in normal B cells and NHL as well as the fact that veltuzumab mediates ADCC more efficiently than epratuzumab.

The bsAbs inhibit lymphoma cells without immobilization (required for epratuzumab) or hypercrosslinking with a secondary antibody (required for veltuzumab). Such direct cytotoxicity is about 50-fold more potent in Daudi Burkitt lymphoma cells than the combination of both parental mAbs in the absence of immobilization or hypercrosslinking In Raji and Ramos cells, 22-(20)-(20) is 8- to 10-fold more potent than 20-(22)-(22), which is in turn 8- to 10-fold more potent than the combination of both parental Abs. Thus, 22-(20)-(20) can be 100-fold more potent than the parental mAbs given in combination in vitro in the absence of other factors, such as effect cells.

Both bsAbs induce extensive translocation of CD22 (as well as CD20) into lipid rafts. Both bsAbs induce strong homotypic adhesion in lymphoma cells, whereas under the same conditions the parental mAbs are ineffective, indicating that crosslinking CD20 and CD22 leads to homotypic adhesion, which may contribute to the enhanced in vitro cytotoxicity.

Pk analyses show that the circulating half-life of the bsAbs in mice is 2-3-fold shorter than that of the parental mAbs. Biodistribution studies in mice show that both bsAbs have tissue uptakes similar to veltuzumab and epratuzumab, indicating that the bsAbs are not cleared more rapidly than their parental mAbs because of increased uptake in normal tissues.

In vivo studies in Daudi xenografts reveal 20-(22)-(22), despite having a shorter serum half-life, had anti-tumor efficacy comparable to equimolar veltuzumab. Although 22-(20)-(20) is less potent than 20-(22)-(22), it is still more effective than epratuzumab and the control bsAbs. The greatly enhanced direct toxicity of the bsAbs correlates with their ability to alter the basal expression of various intracellular proteins involved in regulating cell growth, survival, and apoptosis, with the net outcome leading to cell death. In an ex vivo setting, both 22-(20)-(20) and 20-(22)-(22) deplete NHL cells as well as normal B cells from whole blood of healthy volunteers.

Because Pk analyses revealed that the circulating half-life of the C_{H}3-HexAbs in mice is 2-3-fold shorter than that of the parental mAbs, we have developed the alternative Cₖ-HexAb format, with the goal of improving the Pk. The studies in Example 19 above indicate that the increased rate of blood clearance observed for the C_{H}3-based HexAbs is due to the location of the additional Fab groups at the end of the Fc, interfering with the binding (and/or release) of the neonatal Fc receptor (FcRn), which is responsible for recirculation of IgG following endocytosis, resulting in greatly extended Pk. Indeed, 22*-(20)-(20) exhibited markedly superior Pk compared to 22-(20)-(20) (Example 19). Following subcutaneous injection in normal mice, 22*-(20)-(20) achieved a two-fold greater Cₘₐₓ and three-fold longer circulating half-life, resulting in a three-fold greater area under the curve, compared to 22-(20)-(20). Additionally, 22*-(20)-(20) was found to be highly stable in vivo over the entire 5-day Pk study. This was evident because two different ELISA formats, one of which detects any form of epratuzumab, and the other quantifying only intact 22*-(20)-(20), generated essentially overlapping Pk curves.

Use in SLE

The 22*-(20)-(20) DNL™ construct is selected for therapeutic use in SLE. 22*-(20)-(20) is derived from veltuzumab, the humanized anti-CD20 monoclonal antibody (mAb) and epratuzumab, the humanized anti-CD22 mAb, to form a covalent conjugate with four Fab fragments of veltuzumab attached to one IgG of epratuzumab (see Example 19). Both epratuzumab and veltuzumab have shown clinical activity in autoimmune disease and combination therapy with both mAbs will be more effective than either as monotherapies. A more potent therapy, using two different mechanisms of action (B-cell depletion by anti-CD20 mAb and B-cell modulation by anti-CD22 mAb), is accomplished by using a bispecific antibody capable of targeting both CD20 and CD22 that is more convenient and economical than administering two different mAbs sequentially, which presently requires patients to be infused for many hours in each treatment session.

Use of 22*-(20)-(20) as a therapeutic agent for SLE is evaluated in an SCID mouse model, in which animals are engrafted with peripheral blood lymphocytes (PBL) from SLE patients (Mauermann et al., 2004, Clin Exp Immunol 137:513-520). The efficacy of the bsAb is compared to epratuzumab and veltuzumab independently and in combination by monitoring the serum level of anti-dsDNA antibody, a hallmark of SLE.

Blood samples are collected from SLE patients. For engraftment, 3×10⁷ PBLs obtained from individual SLE patients are injected intraperitoneally (i.p.) into an 8-10 week old female SCID mouse. Thus, each animal represents an individual lupus patient. Approximately two-thirds of the mice have successful engraftment, with evidence of human antibody production in concentrations ≧100 μg/mL within 2 weeks, with peak production within 4 weeks. Mice having evidence of engraftment, are used for treatment. To monitor the effect of treatment, mice are bled on days 24, 34, 44, and 54 and the sera are tested by ELISA or Protein-A HPLC for the presence of total hIgG, anti-dsDNA (measurement of lupus disease state) and anti-tetanus toxoid antibodies (to demonstrate functional human humoral immune system).

Human anti-dsDNA antibodies in the recipient mouse sera, as an indicator of SLE, are determined using maxisorb 96-well microtitre plates coated with poly L-lysine (5 mg/ml, Sigma, St. Louis, Mo., USA), followed by coating with lambda phage dsDNA (5 mg/well, Boehringer Mannheim, Germany). Plates are blocked with 10% fetal calf serum (FCS) in PBS, and incubated with mouse sera (diluted 1:5-1:40) for 2 h. Bound anti-dsDNA is detected with a goat antihuman IgG antibody conjugated to horseradish peroxidase (Jackson).

The effect of using the 22*-(20)-(20) DNL™ on SLE mice is examined. Prior studies with the 22-(20)-(20) bsAb in vitro found it was effective in killing human B-cell lymphoma cell lines at concentrations of ˜1 nM (˜350 μg/mL), and in vivo, three 10-μg doses of 22-(20)-(20) in 1 week controlled the outgrowth of IV implanted Daudi B-cell lymphoma cell line in SCID mice (Rossi et al., 2009, Blood 113:6161-6171). However, the bsAbs were less effective in killing normal B-cells ex vivo (not shown). Based on these results, SLE-engrafted SCID mice are treated initially with 400 μg of 22*-(20)-(20) i.p. twice weekly for 2 weeks (starting on day 14). If titers return, a second cycle of treatment is initiated, continuing until study termination on day 60. If disease control at 400 μg is insufficient after 2 weeks of treatment, treatment in subsequent groups of animals given 400 μg is uninterrupted for 4 sequential weeks. If disease control is significantly improved after 2 weeks with 400 μg, a lower dose of 40 μg using the same twice weekly schedule for 2 weeks is followed by an observation period. Equal numbers of animals receive only the excipient (buffer) dosing so that a baseline for disease progression is established. Our goal is to establish a treatment protocol using a minimum dose that significantly decreases antibody production, proteinuria, and evidence of renal damage. In addition to the buffer control, the effects are determined of the parental mAbs of 22*-(20)-(20), epratuzumab, veltuzumab and a combination of epratuzumab and veltuzumab IgG, as well as veltuzumab-DDD2 (bivalent Fab), each given at equal molar amounts and following the same dosing schedule as the 22*-(20)-(20) test group.

The primary comparator among treatment groups is the change in anti-dsDNA antibody serum titer following treatment. Based on the results of Maurermann et al. using this model system, the anti-dsDNA titer in control mice peaks at 30-40 days, before slowly declining. Successful therapy results in a much lower Cₘₐₓ and more rapid decline in anti-dsDNA titer, to levels below those at the onset of therapy. The Cₘₐₓ and the change in anti-dsDNA titer from day 14 to day 70 (ΔC_{70/14}) are measured for each animal. At the end of the therapy study, animals are assessed for proteinuria and inflammatory glomerulonephritis as additional measurements of disease progression or control.

It is observed that SLE mice treated with a 400 μg dose of 22*-(20)-(20) twice weekly for four weeks show a significant decrease in anti-dsDNA titer, with lower levels of proteinuria and inflammatory glomerulonephritis, compared to the buffer control, either epratuzumab or veltuzumab administered alone, or the combination of epratuzumab and veltuzumab, when administered at the same molar dosages and schedules as 22*-(20)-(20).

### Example 21

### General Techniques for DOCK-AND-LOCK™

The general techniques discussed below were used to generate DNL™ complexes with AD or DDD moieties attached to the C-terminal end of the antibody heavy chain. Light chain appended AD moieties were constructed as described in Example 19 above. With the exception of superior Pk, in vivo stability and improved efficacy, the Cₖ DNL™ constructs were found to function similarly to their C_{H} counterparts.

Expression Vectors—

The plasmid vector pdHL2 has been used to produce a number of antibodies and antibody-based constructs. See Gillies et al., J Immunol Methods (1989), 125:191-202; Losman et al., Cancer (Phila) (1997), 80:2660-6. The di-cistronic mammalian expression vector directs the synthesis of the heavy and light chains of IgG. The vector sequences are mostly identical for many different IgG-pdHL2 constructs, with the only differences existing in the variable domain (V_{H} and V_{L}) sequences. Using molecular biology tools known to those skilled in the art, these IgG expression vectors can be converted into Fab-DDD or Fab-AD expression vectors.

To generate Fab-DDD expression vectors, the coding sequences for the hinge, CH2 and CH3 domains of the heavy chain were replaced with a sequence encoding the first 4 residues of the hinge, a 14 residue Gly-Ser linker and a DDD moiety, such as the first 44 residues of human RIIα (referred to as DDD1, SEQ ID NO:1). To generate Fab-AD expression vectors, the sequences for the hinge, CH2 and CH3 domains of IgG were replaced with a sequence encoding the first 4 residues of the hinge, a 15 residue Gly-Ser linker and an AD moiety, such as a 17 residue synthetic AD called AKAP-IS (referred to as AD1, SEQ ID NO:3), which was generated using bioinformatics and peptide array technology and shown to bind RIIα dimers with a very high affinity (0.4 nM). See Alto, et al. Proc. Natl. Acad. Sci., U.S.A (2003), 100:4445-50. Two shuttle vectors were designed to facilitate the conversion of IgG-pdHL2 vectors to either Fab-DDD1 or Fab-AD1 expression vectors, as described below.

### Preparation of CH1—

The CH1 domain was amplified by PCR using the pdHL2 plasmid vector as a template. The left PCR primer consisted of the upstream (5′) end of the CH1 domain and a SacII restriction endonuclease site, which is 5′ of the CH1 coding sequence. The right primer consisted of the sequence coding for the first 4 residues of the hinge (PKSC, SEQ ID NO:127) followed by four glycines and a serine, with the final two codons (GS) comprising a Bam HI restriction site. The 410 bp PCR amplimer was cloned into the PGEMT® PCR cloning vector (PROMEGA®, Inc.) and clones were screened for inserts in the T7 (5′) orientation.

A duplex oligonucleotide was synthesized to code for the amino acid sequence of DDD1 preceded by 11 residues of the linker peptide, with the first two codons comprising a BamHI restriction site. A stop codon and an EagI restriction site are appended to the 3′ end. The encoded polypeptide sequence is shown below.

(SEQ ID NO: 128)
GSGGGGSGGGGSHIQIPPGLTELLQGYTVEVLRQQPPDLVEFAVEYFTRL
REARA

Two oligonucleotides, designated RIIA1-44 top and RIIA1-44 bottom, which overlap by 30 base pairs on their 3′ ends, were synthesized and combined to comprise the central 154 base pairs of the 174 bp DDD1 sequence. The oligonucleotides were annealed and subjected to a primer extension reaction with Taq polymerase. Following primer extension, the duplex was amplified by PCR. The amplimer was cloned into PGEMT® and screened for inserts in the T7 (5′) orientation.

A duplex oligonucleotide was synthesized to code for the amino acid sequence of AD1 preceded by 11 residues of the linker peptide with the first two codons comprising a BamHI restriction site. A stop codon and an EagI restriction site are appended to the 3′ end. The encoded polypeptide sequence is shown below.

(SEQ ID NO: 129)
GSGGGGSGGGGSQIEYLAKQIVDNAIQQA

Two complimentary overlapping oligonucleotides encoding the above peptide sequence, designated AKAP-IS Top and AKAP-IS Bottom, were synthesized and annealed. The duplex was amplified by PCR. The amplimer was cloned into the PGEMT® vector and screened for inserts in the T7 (5′) orientation.

Ligating DDD1 with CH1—

A 190 bp fragment encoding the DDD1 sequence was excised from PGEMT® with BamHI and NotI restriction enzymes and then ligated into the same sites in CH1-PGEMT® to generate the shuttle vector CH1-DDD1-PGEMT®.

Ligating AD1 with CH1—

A 110 bp fragment containing the AD1 sequence was excised from PGEMT® with BamHI and NotI and then ligated into the same sites in CH1-PGEMT® to generate the shuttle vector CH1-AD1-PGEMT®.

With this modular design either CH1-DDD1 or CH1-AD1 can be incorporated into any IgG construct in the pdHL2 vector. The entire heavy chain constant domain is replaced with one of the above constructs by removing the SacII/EagI restriction fragment (CH1-CH3) from pdHL2 and replacing it with the SacII/EagI fragment of CH1-DDD1 or CH1-AD1, which is excised from the respective PGEMT® shuttle vector.

C-DDD2-Fd-hMN-14-pdHL2—

C-DDD2-Fd-hMN-14-pdHL2 is an expression vector for production of C-DDD2-Fab-hMN-14, which possesses a dimerization and docking domain sequence of DDD2 (SEQ ID NO:2) appended to the carboxyl terminus of the Fd of hMN-14 via a 14 amino acid residue Gly/Ser peptide linker. The fusion protein secreted is composed of two identical copies of hMN-14 Fab held together by non-covalent interaction of the DDD2 domains.

The expression vector was engineered as follows. Two overlapping, complimentary oligonucleotides, which comprise the coding sequence for part of the linker peptide and residues 1-13 of DDD2, were made synthetically. The oligonucleotides were annealed and phosphorylated with T4 PNK, resulting in overhangs on the 5′ and 3′ ends that are compatible for ligation with DNA digested with the restriction endonucleases BamHI and PstI, respectively.

The duplex DNA was ligated with the shuttle vector CH1-DDD1-PGEMT®, which was prepared by digestion with BamHI and PstI, to generate the shuttle vector CH1-DDD2-PGEMT®. A 507 bp fragment was excised from CH1-DDD2-PGEMT® with SacII and EagI and ligated with the IgG expression vector hMN-14(I)-pdHL2, which was prepared by digestion with SacII and EagI. The final expression construct was designated C-DDD2-Fd-hMN-14-pdHL2. Similar techniques have been utilized to generated DDD2-fusion proteins of the Fab fragments of a number of different humanized antibodies.

h679-Fd-AD2-pdHL2—

h679-Fab-AD2, was designed to pair to C-DDD2-Fab-hMN-14. h679-Fd-AD2-pdHL2 is an expression vector for the production of h679-Fab-AD2, which possesses an anchoring domain sequence of AD2 (SEQ ID NO:4) appended to the carboxyl terminal end of the CH1 domain via a 14 amino acid residue Gly/Ser peptide linker AD2 has one cysteine residue preceding and another one following the anchor domain sequence of AD 1.

The expression vector was engineered as follows. Two overlapping, complimentary oligonucleotides (AD2 Top and AD2 Bottom), which comprise the coding sequence for AD2 and part of the linker sequence, were made synthetically. The oligonucleotides were annealed and phosphorylated with T4 PNK, resulting in overhangs on the 5′ and 3′ ends that are compatible for ligation with DNA digested with the restriction endonucleases BamHI and SpeI, respectively.

The duplex DNA was ligated into the shuttle vector CH1-AD1-PGEMT®, which was prepared by digestion with BamHI and SpeI, to generate the shuttle vector CH1-AD2-PGEMT®. A 429 base pair fragment containing CH1 and AD2 coding sequences was excised from the shuttle vector with SacII and EagI restriction enzymes and ligated into h679-pdHL2 vector that prepared by digestion with those same enzymes. The final expression vector is h679-Fd-AD2-pdHL2.

Generation of TF2 DNL™ Construct—

A trimeric DNL™ construct designated TF2 was obtained by reacting C-DDD2-Fab-hMN-14 with h679-Fab-AD2. A pilot batch of TF2 was generated with >90% yield as follows. Protein L-purified C-DDD2-Fab-hMN-14 (200 mg) was mixed with h679-Fab-AD2 (60 mg) at a 1.4:1 molar ratio. The total protein concentration was 1.5 mg/ml in PBS containing 1 mM EDTA. Subsequent steps involved TCEP reduction, HIC chromatography, DMSO oxidation, and IMP 291 affinity chromatography. Before the addition of TCEP, SE-HPLC did not show any evidence of a₂b formation. Addition of 5 mM TCEP rapidly resulted in the formation of a₂b complex consistent with a 157 kDa protein expected for the binary structure. TF2 was purified to near homogeneity by IMP 291 affinity chromatography (not shown). IMP 291 is a synthetic peptide containing the HSG hapten to which the 679 Fab binds (Rossi et al., 2005, Clin Cancer Res 11:7122s-29s). SE-HPLC analysis of the IMP 291 unbound fraction demonstrated the removal of a₄, a₂ and free kappa chains from the product (not shown).

The functionality of TF2 was determined by BIACORE® assay. TF2, C-DDD1-hMN-14+h679-AD1 (used as a control sample of noncovalent a₂b complex), or C-DDD2-hMN-14+h679-AD2 (used as a control sample of unreduced a₂ and b components) were diluted to 1 .mu.g/ml (total protein) and passed over a sensorchip immobilized with HSG. The response for TF2 was approximately two-fold that of the two control samples, indicating that only the h679-Fab-AD component in the control samples would bind to and remain on the sensorchip. Subsequent injections of WI2 IgG, an anti-idiotype antibody for hMN-14, demonstrated that only TF2 had a DDD-Fab-hMN-14 component that was tightly associated with h679-Fab-AD as indicated by an additional signal response. The additional increase of response units resulting from the binding of WI2 to TF2 immobilized on the sensorchip corresponded to two fully functional binding sites, each contributed by one subunit of C-DDD2-Fab-hMN-14. This was confirmed by the ability of TF2 to bind two Fab fragments of WI2 (not shown).

Production of TF10 DNL™ Construct—

A similar protocol was used to generate a trimeric TF10 DNL™ construct, comprising two copies of a C-DDD2-Fab-hPAM4 and one copy of C-AD2-Fab-679. The TF10 bispecific ([hPAM4]₂×h679) antibody was produced using the method disclosed for production of the (anti CEA)₂×anti HSG bsAb TF2, as described above. The TF10 construct bears two humanized PAM4 Fabs and one humanized 679 Fab.

The two fusion proteins (hPAM4-DDD2 and h679-AD2) were expressed independently in stably transfected myeloma cells. The tissue culture supernatant fluids were combined, resulting in a two-fold molar excess of hPAM4-DDD2. The reaction mixture was incubated at room temperature for 24 hours under mild reducing conditions using 1 mM reduced glutathione. Following reduction, the reaction was completed by mild oxidation using 2 mM oxidized glutathione. TF10 was isolated by affinity chromatography using IMP291-affigel resin, which binds with high specificity to the h679 Fab.

### Example 22

### Production of AD- and DDD-Linked Fab and IgG Fusion Proteins from Multiple Antibodies

Using the techniques described in the preceding Examples, the IgG and Fab fusion proteins shown in Table 13 were constructed and incorporated into DNL™ constructs. The fusion proteins retained the antigen-binding characteristics of the parent antibodies and the DNL™ constructs exhibited the antigen-binding activities of the incorporated antibodies or antibody fragments.

TABLE 13
Fusion proteins comprising IgG or Fab

| Fusion Protein | Binding Specificity |
|---|---|
| C-AD1-Fab-h679 | HSG |
| C-AD2-Fab-h679 | HSG |
| C-(AD)₂-Fab-h679 | HSG |
| C-AD2-Fab-h734 | Indium-DTPA |
| C-AD2-Fab-hA20 | CD20 |
| C-AD2-Fab-hA20L | CD20 |
| C-AD2-Fab-hL243 | HLA-DR |
| C-AD2-Fab-hLL2 | CD22 |
| N-AD2-Fab-hLL2 | CD22 |
| C-AD2-IgG-hMN-14 | CEACAM5 |
| C-AD2-IgG-hR1 | IGF-1R |
| C-AD2-IgG-hRS7 | EGP-1 |
| C-AD2-IgG-hPAM4 | MUC |
| C-AD2-IgG-hLL1 | CD74 |
| C-DDD1-Fab-hMN-14 | CEACAM5 |
| C-DDD2-Fab-hMN-14 | CEACAM5 |
| C-DDD2-Fab-h679 | HSG |
| C-DDD2-Fab-hA19 | CD19 |
| C-DDD2-Fab-hA20 | CD20 |
| C-DDD2-Fab-hAFP | AFP |
| C-DDD2-Fab-hL243 | HLA-DR |
| C-DDD2-Fab-hLL1 | CD74 |
| C-DDD2-Fab-hLL2 | CD22 |
| C-DDD2-Fab-hMN-3 | CEACAM6 |
| C-DDD2-Fab-hMN-15 | CEACAM6 |
| C-DDD2-Fab-hPAM4 | MUC |
| C-DDD2-Fab-hR1 | IGF-1R |
| C-DDD2-Fab-hRS7 | EGP-1 |
| N-DDD2-Fab-hMN-14 | CEACAM5 |

### Example 23

### Production and Use of a DNL™ Construct Comprising Two Different Antibody Moieties and a Cytokine

In certain embodiments, trimeric DNL™ constructs may comprise three different effector moieties, for example two different antibody moieties and a cytokine moiety. We report here the generation and characterization of the first bispecific MAb-IFNα, designated 20-C₂-2b, which comprises two copies of IFN-α2b and a stabilized F(ab)₂ of hL243 (humanized anti-HLA-DR; IMMU-114) site-specifically linked to veltuzumab (humanized anti-CD20). In vitro, 20-C₂-2b inhibited each of four lymphoma and eight myeloma cell lines, and was more effective than monospecific CD20-targeted MAb-IFNα or a mixture comprising the parental antibodies and IFNα in all but one (HLA-DR⁻/CD20⁻) myeloma line (not shown), suggesting that 20-C2-2b should be useful in the treatment of various hematopoietic disorders. The 20-C2-2b displayed greater cytotoxicity against KMS12-BM (CD20 VHLA-DR⁺ myeloma) than monospecific MAb-IFNα that targets only HLA-DR or CD20 (not shown), indicating that all three components in 20-C2-2b can contribute to toxicity. Our findings indicate that a given cell's responsiveness to MAb-IFNα depends on its sensitivity to IFNα and the specific antibodies, as well as the expression and density of the targeted antigens.

Because 20-C2-2b has antibody-dependent cellular cytotoxicity (ADCC), but not CDC, and can target both CD20 and HLA-DR, it is useful for therapy of a broad range of hematopoietic disorders that express either or both antigens.

Antibodies—

The abbreviations used in the following discussion are: 20 (C_{H}3-AD2-IgG-v-mab, anti-CD20 IgG DNL™ module); C2 (C_{H}1-DDD2-Fab-hL243, anti-HLA-DR Fab₂ DNL™ module); 2b (dimeric IFNα2B-DDD2 DNL™ module); 734 (anti-in-DTPA IgG DNL™ module used as non-targeting control). The following MAbs were provided by Immunomedics, Inc.: veltuzumab or v-mab (anti-CD20 IgG₁), hL243γ4p (Immu-114, anti-HLA-DR IgG₄), a murine anti-IFNα MAb, and rat anti-idiotype MAbs to v-mab (WR2) and hL243 (WT).

DNL™ Constructs—

Monospecific MAb-IFNα (20-2b-2b,734-2b-2b and C2-2b-2b) and the bispecific HexAb (20-C2-C2) were generated by combination of an IgG-AD2-module with DDD2-modules using the DNL™ method, as described in the preceding Examples. The 734-2b-2b, which comprises tetrameric IFNα2b and MAb h734 [anti-Indium-DTPA IgG₁], was used as a non-targeting control MAb-IFNα.

The construction of the mammalian expression vector as well as the subsequent generation of the production clones and the purification of C_{H}3-AD2-IgG-v-mab are disclosed in the preceding Examples. The expressed recombinant fusion protein has the AD2 peptide linked to the carboxyl terminus of the C_{H}3 domain of v-mab via a 15 amino acid long flexible linker peptide. Co-expression of the heavy chain-AD2 and light chain polypeptides results in the formation of an IgG structure equipped with two AD2 peptides. The expression vector was transfected into Sp/ESF cells (an engineered cell line of Sp2/0) by electroporation. The pdHL2 vector contains the gene for dihydrofolate reductase, thus allowing clonal selection, as well as gene amplification with methotrexate (MTX). Stable clones were isolated from 96-well plates selected with media containing 0.2 μM MTX. Clones were screened for C_{H}3-AD2-IgG-vmab productivity via a sandwich ELISA. The module was produced in roller bottle culture with serum-free media.

The DDD-module, IFNα2b-DDD2, was generated as discussed above by recombinant fusion of the DDD2 peptide to the carboxyl terminus of human IFNα2b via an 18 amino acid long flexible linker peptide. As is the case for all DDD-modules, the expressed fusion protein spontaneously forms a stable homodimer.

The C_{H}1-DDD2-Fab-hL243 expression vector was generated from hL243-IgG-pdHL2 vector by excising the sequence for the C_{H}1-Hinge-C_{H}2-C_{H}3 domains with SacII and EagI restriction enzymes and replacing it with a 507 bp sequence encoding C_{H}1-DDD2, which was excised from the C-DDD2-hMN-14-pdHL2 expression vector with the same enzymes. Following transfection of C_{H}1-DDD2-Fab-hL243-pdHL2 into Sp/ESF cells by electroporation, stable, MTX-resistant clones were screened for productivity via a sandwich ELISA using 96-well microtiter plates coated with mouse anti-human kappa chain to capture the fusion protein, which was detected with horseradish peroxidase-conjugated goat anti-human Fab. The module was produced in roller bottle culture.

Roller bottle cultures in serum-free H—SFM media and fed-batch bioreactor production resulted in yields comparable to other IgG-AD2 modules and cytokine-DDD2 modules generated to date. C_{H}3-AD2-IgG-v-mab and IFNα2b-DDD2 were purified from the culture broths by affinity chromatography using MABSELECT™ (GE Healthcare) and HIS-SELECT® HF Nickel Affinity Gel (Sigma), respectively, as described previously (Rossi et al., Blood 2009, 114:3864-71). The culture broth containing the C_{H}1-DDD2-Fab-hL243 module was applied directly to KAPPASELECT® affinity gel (GE-Healthcare), which was washed to baseline with PBS and eluted with 0.1 M Glycine, pH 2.5.

Generation of 20-C2-2b by DNL™—

Three DNL™ modules (C_{H}3-AD2-IgG-v-mab, C_{H}1-DDD2-Fab-hL243, and IFN-α2b-DDD2) were combined in equimolar quantities to generate the bsMAb-IFNα, 20-C2-2b. Following an overnight docking step under mild reducing conditions (1 mM reduced glutathione) at room temperature, oxidized glutathione was added (2 mM) to facilitate disulfide bond formation (locking) The 20-C2-2b was purified to near homogeneity using three sequential affinity chromatography steps. Initially, the DNL™ mixture was purified with Protein A (MABSELECT™), which binds the C_{H}3-AD2-IgG-v-MAb group and eliminates un-reacted IFNα2b-DDD2 or C_{H}1-DDD2-Fab-hL243. The Protein A-bound material was further purified by IMAC using HIS-SELECT® HF Nickel Affinity Gel, which binds specifically to the IFNα2b-DDD2 moiety and eliminates any constructs lacking this group. The final process step, using an hL243-anti-idiotype affinity gel removed any molecules lacking C_{H}1-DDD2-Fab-hL243.

The skilled artisan will realize that affinity chromatography may be used to purify DNL™ complexes comprising any combination of effector moieties, so long as ligands for each of the three effector moieties can be obtained and attached to the column material. The selected DNL™ construct is the one that binds to each of three columns containing the ligand for each of the three effector moieties and can be eluted after washing to remove unbound complexes.

The following Example is representative of several similar preparations of 20-C2-2b. Equimolar amounts of C_{H}3-AD2-IgG-v-mab (15 mg), C_{H}1-DDD2-Fab-hL243 (12 mg), and IFNα2b-DDD2 (5 mg) were combined in 30-mL reaction volume and 1 mM reduced glutathione was added to the solution. Following 16 h at room temperature, 2 mM oxidized glutathione was added to the mixture, which was held at room temperature for an additional 6 h. The reaction mixture was applied to a 5-mL Protein A affinity column, which was washed to baseline with PBS and eluted with 0.1 M Glycine, pH 2.5. The eluate, which contained ˜20 mg protein, was neutralized with 3 M Tris-HCl, pH 8.6 and dialyzed into HIS-SELECT® binding buffer (10 mM imidazole, 300 mM NaCl, 50 mM NaH₂PO₄, pH 8.0) prior to application to a 5-mL HIS-SELECT® IMAC column. The column was washed to baseline with binding buffer and eluted with 250 mM imidazole, 150 mM NaCl, 50 mM NaH₂PO₄, pH 8.0.

The IMAC eluate, which contained ˜11.5 mg of protein, was applied directly to a WP (anti-hL243) affinity column, which was washed to baseline with PBS and eluted with 0.1 M glycine, pH 2.5. The process resulted in 7 mg of highly purified 20-C2-2b. This was approximately 44% of the theoretical yield of 20-C2-2b, which is 50% of the total starting material (16 mg in this example) with 25% each of 20-2b-2b and 20-C2-C2 produced as side products.

Generation and characterization of 20-C2-2b—

The bispecific MAb-IFNα was generated by combining the IgG-AD2 module, C_{H}3-AD2-IgG-v-mab, with two different dimeric DDD-modules, C_{H}1-DDD2-Fab-hL243 and IFNα2b-DDD2. Due to the random association of either DDD-module with the two AD2 groups, two side-products, 20-C2-C2 and 20-2b-2b are expected to form, in addition to 20-C2-2b.

Non-reducing SDS-PAGE (not shown) resolved 20-C2-2b (˜305 kDa) as a cluster of bands positioned between those of 20-C2-C2 (˜365 kDa) and 20-2b-2b (255 kDa). Reducing SDS-PAGE resolved the five polypeptides (v-mab HC-AD2, hL243 Fd-DDD2, IFNα2b-DDD2 and co-migrating v-mab and hL243 kappa light chains) comprising 20-C2-2b (not shown). IFNα2b-DDD2 and hL243 Fd-DDD2 are absent in 20-C2-C2 and 20-2b-2b. MABSELECT™ binds to all three of the major species produced in the DNL™ reaction, but removes any excess IFNα2b-DDD2 and C_{H}1-DDD2-Fab-hL243. The HIS-SELECT® unbound fraction contained mostly 20-C2-C2 (not shown). The unbound fraction from WT affinity chromatography comprised 20-2b-2b (not shown). Each of the samples was subjected to SE-HPLC and immunoreactivity analyses, which corroborated the results and conclusions of the SDS-PAGE analysis.

Following reduction of 20-C2-2b, its five component polypeptides were resolved by RP-HPLC and individual ESI-TOF deconvoluted mass spectra were generated for each peak (not shown). Native, but not bacterially-expressed recombinant IFNα2, is O-glycosylated at Thr-106 (Adolf et al., Biochem J 1991; 276 (Pt 2):511-8). We determined that ˜15% of the polypeptides comprising the IFNα2b-DDD2 module are O-glycosylated and can be resolved from the non-glycosylated polypeptides by RP-HPLC and SDS-PAGE (not shown). LC/MS analysis of 20-C2-2b identified both the O-glycosylated and non-glycosylated species of IFNα2b-DDD2 with mass accuracies of 15 ppm and 2 ppm, respectively (not shown). The observed mass of the O-glycosylated form indicates an O-linked glycan having the structure NeuGc-NeuGc-Gal-GalNAc, which was also predicted (<1 ppm) for 20-2b-2b (not shown). LC/MS identified both v-mab and hL243 kappa chains as well as hL243-Fd-DDD2 (not shown) as single, unmodified species, with observed masses matching the calculated ones (<35 ppm). Two major glycoforms of v-mab HC-AD2 were identified as having masses of 53,714.73 (70%) and 53,877.33 (30%), indicating G0F and G1F N-glycans, respectively, which are typically associated with IgG (not shown). The analysis also confirmed that the amino terminus of the HC-AD2 is modified to pyroglutamate, as predicted for polypeptides having an amino terminal glutamine.

SE-HPLC analysis of 20-C2-2b resolved a predominant protein peak with a retention time (6.7 min) consistent with its calculated mass and between those of the larger 20-C2-C2 (6.6 min) and smaller 20-2b-2b (6.85 min), as well as some higher molecular weight peaks that likely represent non-covalent dimers formed via self-association of IFNα2b (not shown).

Immunoreactivity assays demonstrated the homogeneity of 20-C2-2b with each molecule containing the three functional groups (not shown). Incubation of 20-C2-2b with an excess of antibodies to any of the three constituent modules resulted in quantitative formation of high molecular weight immune complexes and the disappearance of the 20-C2-2b peak (not shown). The HIS-SELECT® and WT affinity unbound fractions were not immunoreactive with WT and anti-IFNα, respectively (not shown). The MAb-IFNα showed similar binding avidity to their parental MAbs (not shown).

IFNα Biological Activity—

The specific activities for various MAb-IFNα were measured using a cell-based reporter gene assay and compared to peginterferon alfa-2b (not shown). Expectedly, the specific activity of 20-C2-2b (2454 IU/pmol), which has two IFNα2b groups, was significantly lower than those of 20-2b-2b (4447 IU/pmol) or 734-2b-2b (3764 IU/pmol), yet greater than peginterferon alfa-2b (P<0.001) (not shown). The difference between 20-2b-2b and 734-2b-2b was not significant. The specific activity among all agents varies minimally when normalized to IU/pmol of total IFNα. Based on these data, the specific activity of each IFNα2b group of the MAb-IFNα is approximately 30% of recombinant IFNα2b (4000 IU/pmol).

In the ex-vivo setting, the 20-C2-2b DNL™ construct depleted lymphoma cells more effectively than normal B cells and had no effect on T cells (not shown). However, it did efficiently eliminate monocytes (not shown). Where v-mab had no effect on monocytes, depletion was observed following treatment with hL243α4p and MAb-IFNα, with 20-2b-2b and 734-2b-2b exhibiting similar toxicity (not shown). Therefore, the predictably higher potency of 20-C2-2b is attributed to the combined actions of anti-HLA-DR and IFNα, which may be augmented by HLA-DR targeting. These data suggest that monocyte depletion may be a pharmacodynamic effect associated anti-HLA-DR as well as IFNα therapy; however, this side affect would likely be transient because the monocyte population should be repopulated from hematopoietic stem cells.

The skilled artisan will realize that the approach described here to produce and use bispecific immunocytokine, or other DNL™ constructs comprising three different effector moieties, may be utilized with any combinations of antibodies, antibody fragments, cytokines or other effectors that may be incorporated into a DNL™ construct, for example the combination of anti-CD20 and anti-CD22 with IFNα2b.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the invention.

What is claimed is:

1. A method of treating a B-cell associated disease, selected from the group consisting of B-cell malignancy, autoimmune disease and immune dysfunction disease, comprising administering to a subject with the disease an antibody that binds to B cells, wherein the antibody induces trogocytosis of one or more B-cell surface antigens.

2. The method of claim 1, wherein the antibody induces trogocytosis of one or more B-cell surface antigens when exposed to B cells in vitro in the presence of PBMCs or purified FcγR-positive cells.

3. The method of claim 1, wherein the antibody induces trogocytosis of one or more B-cell surface antigens when exposed to circulating B cells in vivo.

4. The method of claim 1, wherein the antibody induces trogocytosis of one or more B-cell surface antigens selected from the group consisting of CD19, CD20, CD21, CD22, CD79b, CD44, CD62L and β7-integrin.

5. The method of claim 1, wherein the antibody binds to a B-cell antigen selected from the group consisting of CD19, CD20, CD21, CD22, CD79b, CD44, CD62L, CD74, HLA-DR, β7-integrin and BCR.

6. The method of claim 1, wherein the subject is a human subject.

7. The method of claim 1, wherein the antibody is a chimeric, humanized, or human antibody.

8. The method of claim 1, wherein the antibody induces trogocytosis of one or more B-cell antigens, without depleting circulating B cells by more than 50%, when the antibody is administered to a subject.

9. The method of claim 1, wherein the antibody is effective to kill malignant B cells, without depleting circulating normal B cells by more than 50%, when the antibody is administered to a subject with a B-cell leukemia or lymphoma.

10. The method of claim 1, wherein the B-cell malignancy is selected from the group consisting of B-cell leukemia, B-cell lymphoma, Hodgkin's lymphoma, non-Hodgkin's lymphoma, Burkitt lymphoma, mantle cell lymphoma, acute lymphocytic leukemia, chronic lymphocytic leukemia, hairy cell leukemia, multiple myeloma and Waldenstrom's macroglobulinemia.

11. The method of claim 1, wherein the autoimmune disease is selected from the group consisting of acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, ANCA-associated vasculitides, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjögren's syndrome, primary biliary cirrhosis, Hashimoto's thyroiditis, thyrotoxicosis, scleroderma, chronic active hepatitis, polymyositis/dermatomyositis, polychondritis, bullous pemphigoid, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis and fibrosing alveolitis.

12. The method of claim 1, wherein the autoimmune disease is SLE (systemic lupus erythematosus).

13. The method of claim 1, wherein the immune dysfunction disease is selected from the group consisting of graft-versus-host disease, organ transplant rejection, septicemia, sepsis and inflammation.

14. The method of claim 1, further comprising administering a therapeutic agent to the subject.

15. The method of claim 14, wherein the therapeutic agent is selected from the group consisting of a drug, prodrug, immunomodulator, cytokine, chemokine, pro-apoptotic agent, anti-angiogenic agent, tyrosine kinase inhibitor, Bruton kinase inhibitor, sphingosine inhibitor, enzyme, hormone, photoactive agent, siRNA and RNAi.

16. The method of claim 15, wherein the drug is selected from the group consisting of 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celecoxib, chlorambucil, cisplatinum, Cox-2 inhibitors, irinotecan (CPT-11), SN-38, carboplatin, cladribine, camptothecans, cyclophosphamide, cytarabine, dacarbazine, docetaxel, dactinomycin, daunorubicin, doxorubicin, 2-pyrrolinodoxorubicine (2PDOX), pro-2PDOX, cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR (FUdR-dO), fludarabine, flutamide, farnesyl-protein transferase inhibitors, gemcitabine, hydroxyurea, idarubicin, ifosfamide, L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin, PSI-341, raloxifene, semustine, streptozocin, tamoxifen, temazolomide (an aqueous form of DTIC), transplatinum, thalidomide, thioguanine, thiotepa, teniposide, topotecan, uracil mustard, vinorelbine, vinblastine, vincristine and vinca alkaloids.

17. The method of claim 15, wherein the tyrosine kinase inhibitor is selected from the group consisting of canertinib, dasatinib, erlotinib, gefitinib, imatinib, lapatinib, leflunomide, nilotinib, pazopanib, semaxinib, sorafenib, sunitinib, sutent and vatalanib.

18. The method of claim 15, wherein the Bruton kinase inhibitor is selected from the group consisting of PCI-32765 (ibrutinib), PCI-45292, GDC-0834, LFM-A13 and RN486.

19. The method of claim 15, wherein the immunomodulator is selected from the group consisting of a cytokine, a stem cell growth factor, a lymphotoxin, a hematopoietic factor, a colony stimulating factor, erythropoietin, thrombopoietin, tumor necrosis factor-α (TNF-α), TNF-β, granulocyte-colony stimulating factor (G-CSF), granulocyte macrophage-colony stimulating factor (GM-CSF), interferon-α, interferon-β, interferon-λ, interferon-γ, “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, M-CSF, interleukin-1 (IL-1), IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-23, IL-25, LIF, kit-ligand (FLT-3), angiostatin, thrombospondin, endostatin, and lymphotoxin.

20. The method of claim 15, wherein the anti-angiogenic agent is selected from the group consisting of angiostatin, baculostatin, canstatin, maspin, anti-placenta growth factor (anti-P1GF), anti-VEGF, anti-Flk-1 antibody, anti-Flt-1 antibody, anti-Kras antibody, anti-cMET antibody, anti-MIF (macrophage migration-inhibitory factor) antibody, laminin peptide, fibronectin peptide, plasminogen activator inhibitor, tissue metalloproteinase inhibitor, an interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, interferon-lambda, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 and minocycline.

21. The method of claim 1, wherein the antibody is a bispecific antibody.

22. The method of claim 21, wherein the bispecific antibody comprises an IgG antibody and one or more antigen-binding antibody fragments.

23. The method of claim 22, wherein the bispecific antibody comprises an IgG antibody and four antigen-binding antibody fragments.

24. The method of claim 21, wherein the bispecific antibody binds to two different B-cell antigens selected from the group consisting of CD19, CD20, CD21, CD22, CD79b, CD44, CD62L, CD74, HLA-DR, β7-integrin and BCR.

25. The method of claim 21, wherein the bispecific antibody binds to an antigen selected from the group consisting of TNF-alpha, IL6 and CD3.

26. The method of claim 21, wherein the bispecific antibody is an anti-CD22×anti-CD20, anti-CD22×anti-CD19, anti-CD22×anti-CD21, anti-CD22×anti-CD79b, anti-CD22×anti-CD44, anti-CD22×anti-CD62L, anti-CD22×anti-β7-integrin, anti-CD22×anti-BCR, anti-CD22×anti-β7-integrin, anti-CD22×anti-CD74, anti-CD22×anti-HLA-DR, anti-CD22×anti-TNF-alpha, anti-CD22×anti-IL6 or anti-CD22×anti-CD3 antibody.

27. The method of claim 21, wherein the bispecific antibody is an anti-CD20×anti-CD19, anti-CD20×anti-CD21, anti-CD20×anti-CD74, anti-CD20×anti-HLA-DR, anti-CD20×anti-TNF-alpha, anti-CD20×anti-IL6, anti-CD20×anti-CD3, anti-CD19×anti-CD21, anti-CD19×anti-CD74, anti-CD19×anti-HLA-DR, anti-CD19×anti-TNF-alpha, anti-CD19×anti-IL6, anti-CD19×anti-CD3 antibody, anti-CD74×anti-HLA-DR, anti-CD74×anti-TNF-alpha, anti-CD74×anti-IL6, anti-CD74×anti-CD3, anti-HLA-DR×anti-TNF-alpha, anti-HLA-DR×anti-IL6, anti-HLA-DR×anti-CD3, anti-TNF-alpha×anti-IL6, anti-TNF-alpha×anti-CD3, or anti-IL6×anti-CD3 bispecific antibody.

28. The method of claim 21, wherein the bispecific antibody is an anti-CD22×anti-CD20 antibody.

29. The method of claim 21, wherein the bispecific antibody comprises an anti-CD20 antibody or antigen-binding fragment thereof, wherein the anti-CD20 antibody is rituximab or veltuzumab.

30. The method of claim 21, wherein the bispecific antibody comprises an anti-CD22 antibody or antigen-binding fragment thereof, wherein the anti-CD22 antibody is epratuzumab or RFB4.

31. The method of claim 22, wherein the IgG antibody and the antigen-binding antibody fragment are fusion proteins.

32. The method of claim 22, wherein the antibody fragment is selected from the group consisting of F(ab′)₂, F(ab)₂, Fab′, Fab, Fv, scFv and single domain antibody fragments.

33. The method of claim 21, wherein the IgG antibody and antibody fragments are selected from the group consisting of chimeric, humanized, and human antibodies and antibody fragments.

34. The method of claim 21, wherein the bispecific antibody induces trogocytosis of one or more B-cell antigens, without depleting circulating B cells by more than 50%, when the antibody is administered to a subject.

35. The method of claim 21, wherein the bispecific antibody is effective to kill malignant B cells, without depleting circulating normal B cells by more than 50%, when the bispecific antibody is administered to a subject with a B-cell leukemia or lymphoma.

36. The method of claim 21, wherein the bispecific antibody is a complex comprising:
a) a first antibody, wherein the C-terminal end of each light chain of the first antibody is conjugated to an anchor domain (AD) moiety from an A-kinase anchoring protein (AKAP); and
b) an antigen-binding fragment of a second antibody, conjugated to a dimerization and docking domain (DDD) moiety from human protein kinase A (PKA) regulatory subunit RIa, RIβ, RIIα or RIIβ;
wherein two copies of the DDD moiety form a dimer that binds to the AD moiety to form the complex.

37. A complex comprising:
a) a first anti-B-cell antibody, wherein the C-terminal end of each light chain of the first antibody is conjugated to an anchor domain (AD) moiety from an A-kinase anchoring protein (AKAP); and
b) an antigen-binding fragment of a second anti-B-cell antibody, conjugated to a dimerization and docking domain (DDD) moiety from human protein kinase A (PKA) regulatory subunit RIα, RIβ, RIIα or RIIβ;
wherein two copies of the DDD moiety form a dimer that binds to the AD moiety to form the complex.

38. The complex of claim 37, wherein the first and second antibodies bind to B-cell antigens selected from the group consisting of CD19, CD20, CD21, CD22, CD79b, CD44, CD62L and β7-integrin.

39. The complex of claim 37, wherein the first and second antibodies bind to B-cell antigens selected from the group consisting of CD20 and CD22.

40. The complex of claim 37, wherein the complex induces trogocytosis of one or more B-cell antigens when exposed to B cells in vitro in the presence of peripheral blood mononuclear cells (PBMCs) or purified FcγR-positive cells.

41. The complex of claim 40, wherein the complex induces trogocytosis of one or more B-cell antigens selected from the group consisting of CD19, CD20, CD21, CD22, CD79b, CD44, CD62L and β7-integrin.

42. The complex of claim 37, wherein the complex induces trogocytosis of one or more B-cell antigens in vivo when the complex is administered to a subject.

43. The complex of claim 42, wherein the complex induces trogocytosis of one or more B-cell antigens selected from the group consisting of CD19, CD20, CD21, CD22, CD79b, CD44, CD62L and β7-integrin.

44. The complex of claim 39, wherein the antibody that binds to CD22 is selected from the group consisting of epratuzumab and RFB4.

45. The complex of claim 39, wherein the antibody that binds to CD20 is selected from the group consisting of veltuzumab and rituximab.

46. The complex of claim 37, wherein the antibodies are chimeric, humanized, or human antibodies.

47. The complex of claim 37, wherein the first antibody and the antigen-binding fragment of the second antibody are fusion proteins.

48. The complex of claim 42, wherein the complex induces trogocytosis of one or more B-cell antigens without depleting circulating B cells by more than 50% when administered to a subject.

49. The complex of claim 42, wherein the complex is effective to kill malignant B cells without depleting circulating normal B cells by more than 50% when administered to a subject with a B-cell leukemia or lymphoma.

50. The complex of claim 42, wherein trogocytosis of one or more B-cell antigens is effective to treat autoimmune disease or immune system dysfunction when administered to a subject with autoimmune disease or immune system dysfunction.

51. A pharmaceutical composition comprising a complex according to claim 1.
